(19) United States
(12) Patent Application Publication
(10) Pub. No.: US 2012/0122787 A1
(43) Pub. Date: May 17, 2012

(54) METHODS FOR TREATING POLYCYSTIC KIDNEY DISEASE (PKD) OR OTHER CYST FORMING DISEASES

(76) Inventor: Rong Li, Kansas City, MO (US)

(21) Appl. No.: 13/382,978

(22) PCT Filed: Jul. 9, 2010
(86) PCT No.: PCT/US10/41481

§ 371 Date: Jan. 26, 2012

Related U.S. Application Data
(60) Provisional application No. 61/270,636, filed on Jul. 10, 2009.

(51) Int. Cl. A61K 31/165; A61K 31/19; A61P 13/12; A61K 31/18; A61K 38/12; A61K 31/357; A61K 31/47; A61K 31/167; A61K 31/426

(52) U.S. Cl. 514/15.4; 514/619; 514/616; 514/557; 514/370; 514/465; 514/311; 514/603; 514/604

(57) ABSTRACT

The present invention is directed to, inter alia, methods of treating or ameliorating an effect of a polycystic disease. This method include administering to a patient in need thereof an amount of a modulator of a histone deacetylase (HDAC) path-way, which is sufficient to treat or ameliorate an effect of a polycystic disease, particularly a polycystic kidney disease.

### CROSS-REFERENCE TO RELATED APPLICATIONS

This application claims benefit to U.S. Provisional Patent Application Ser. No. 61/270,626, filed Jul. 10, 2009, the entire content of which is hereby incorporated by reference as if recited in full herein.

### FIELD OF INVENTION

The present invention relates, inter alia, to methods for treating or ameliorating the effects of a polycystic disease, such as for example, polycystic kidney disease (PKD).

### INCORPORATION BY REFERENCE OF SEQUENCE LISTING

This application contains references to amino acids and/or nucleic acid sequences that have been filed concurrently herewith as sequence listing text file “033342new.txt”, file size of 17.4 KB, created on Jul. 1, 2010. The aforementioned sequence listing is hereby incorporated by reference in its entirety pursuant to 37 C.F.R. §1.52(e)(5).

### BACKGROUND OF INVENTION

Polycystic kidney disease (PKD) is a common inherited human nephropathy that affects approximately 1:1000 of the worldwide population and is the third-most common cause of end stage renal failure. It is characterized by extensive renal cyst formation as a result of altered epithelial cell morphology and proliferation. Renal cysts progressively develop in patients afflicted with PKD, and are associated with complications such as cyst infection and hemorrhage, renal stones, and pain. PKD largely results from the Autosomal Dominant Polycystic Kidney Disease (ADPKD), which is attributed to heterozygous mutations in either the Pkd1 or Pkd2 gene. These genes respectively encode the large, membrane-associated proteins Polycystin-1 (PC1) and Polycystin-2 (PC2) proteins. Polycystin-1 and polycystin-2 are components of a receptor-calcium channel complex that has been proposed to play a mechanosensory role on the epithelial lumenal surface in mammalian kidneys. In both human and mouse, loss of polycystin functions leads to development of polycystic kidneys. In PKD, cyst formation and the associated symptoms may be caused by loss of polycystin function.

During renal development, nephron formation relies on coordinated regulation of cell proliferation, cell polarity, differentiation and apoptosis. Epithelial cell organization is disrupted in ADPKD kidneys, where numerous, fluid-filled cysts develop as a result of aberrant cell proliferation, loss of planar cell polarity and transepithelial fluid secretion, and changes of epithelial cell polarity and cytoskeleton (Grantham, 2003; Wilson and Goilav, 2007).

PC-1 is a large G-protein-coupled receptor-like protein with a complex array of functions, and has been shown to bind polycystin-2 (PC-2), a TRP calcium channel, through a COOH-terminal coiled-coil region (Boletta and Germino, 2003). PC-1 and PC-2 localize to a number of cellular compartments, such as the primary cilia, microtubule-based structures that extend from the apical surface of epithelial cells into the tubule lumen (Berbari et al., 2009; Eley et al., 2005), as well as other epithelial surfaces, and PC-2 associates prominently with the ER (reviewed in Wilson and Goilav, 2007). The cilia localization has gained most attention as physical bending of the primary cilia or fluid flow across the apical surface of epithelial cells caused an increase in intracellular Ca²⁺ (Praetorius and Spring, 2001). Later studies showed that polycystins are required for the fluid flow induced Ca²⁺ influx, suggesting that these proteins, possibly through their association with cilia, function as mechanosensors (Grimm et al., 2002; Nauli et al., 2003). However, conditional inactivation of genes required for ciliogenesis in adult mice did not lead to rapid development of cysts despite the loss of primary cilia (Davenport et al., 2007; Patel et al., 2008). These findings cast doubts on whether the loss of mechanosensory function mediated by the primary cilia is responsible for cyst formation.

Despite a lack of clarity on the functional location of the polycystins, loss of polycystins correlates with disruption of flow-dependent intracellular calcium signaling and a reduction in steady-state intracellular Ca²⁺ levels (Nauli et al., 2006; Yamaguchi et al., 2006). Recent studies have demonstrated a role for intracellular Ca²⁺ in cAMP-dependent cell proliferation. cAMP stimulates the proliferation of ADPKD cells in culture but inhibits the proliferation of normal renal cells. Incubation of normal cells with a calcium channel blocker caused a phenotypic switch such that cAMP stimulated cell proliferation. Furthermore, elevation of intracellular calcium prevented cAMP-induced hyper-proliferation of ADPKD cells (Yamaguchi et al., 2006). Interestingly, treatment of Cy/+ rats, a PKD rat model, with a calcium channel blocker increased the progression of PKD, whereas treatment with a calcium mimetic inhibited late-stage cyst growth (Gattone et al., 2009; Nagao et al., 2008). Signaling molecules or transcription factors, such as MAP kinases, STAT3 and Id2, have been implicated in the regulation of cell proliferation downstream of the polycystins (Bhunia et al., 2002; Li et al., 2005b; Nagao et al., 2003; Yamaguchi et al., 2003), however it is unclear if these pathways are directly regulated by calcium and mechanosensory function of polycystins. Furthermore, the importance of fluid flow-induced calcium signaling through polycystins was called into question in a recent study (Köttgen et al., 2008).

In view of the foregoing, there exists, inter alia, a need for elucidating molecular pathways that directly respond to the fluid flow-induced calcium signal and a need for improved methods for treating and/or ameliorating the symptoms of PKD and other cyst-forming diseases.

### SUMMARY OF INVENTION

The present invention is directed, inter alia, to treating and/or ameliorating the effects of PKD and related conditions by regulating HDAC pathway modulators. Accordingly, one embodiment of the present invention is a method of treating or ameliorating an effect of a polycystic disease. This method comprises administering to a patient in need thereof an amount of a modulator of a histone deacetylase (HDAC) pathway, which amount is sufficient to treat or ameliorate an effect of a polycystic disease.

Another embodiment of the present invention is a method of treating or ameliorating an effect of a polycystic kidney disease (PKD). This method comprises administering to a patient in need thereof an amount of an HDAC inhibitor (HDACi) that is sufficient to treat or ameliorate an effect of PKD.

A further embodiment of the present invention is a method of treating or ameliorating an effect of a polycystic kidney disease (PKD). This method comprises administering to a patient in need thereof an amount of an HDAC5 inhibitor that is sufficient to treat or ameliorate an effect of PKD.

These and other aspects of the invention are further disclosed in the detailed description and examples which follow.

### BRIEF DESCRIPTION OF DRAWINGS

The following drawings form part of the present specification and are included to further demonstrate certain aspects of the present invention. The invention may be better understood by reference to one or more of these drawings in combination with the Detailed Description and the Examples presented herein.

FIG. 1 shows that fluid flow induced HDAC5 phosphorylation in MEK cells. FIG. 1A shows an immunoblot analysis of HDAC5 phosphorylation using a phospho-specific antibody against phosphorylated serine 489 of HDAC5 after Pkd1^{+/+} and Pkd1^{-/-} MEK cells were stimulated with fluid flow at 0.2 ml/min for up to 4 hours. FIG. 1B shows quantification of HDAC5 phosphorylation in the above experiment, normalized first against actin and then against the t₀ values. FIG. 1C shows an immunoblot analysis of HDAC5 phosphorylation in response to fluid flow for 4 hours in Pkd1 siRNA or control lentivirus transduced MEK cells. FIG. 1D shows the effects of a PKC activator (12-myristate-13-acetate (PMA)), a PKC inhibitor (GÖ6983), a calcium ionophore (ionomycin (“iono”)) and a calcium channel blocker (GdCl₃) on HDAC5 phosphorylation without or with fluid flow in MEK cells. PMA (Sigma Aldrich) was used at 100 μM, GÖ6983 (Sigma Aldrich) was used at 10 μM, ionomycin (Sigma Aldrich) was used at 1 μM, and GdCl₃ (Sigma Aldrich) was used at 20 μM.

FIG. 2 shows that fluid flow induced HDAC5 nuclear export in MEK cells. FIG. 2A shows Pkd1^{+/+} and Pkd1^{-/-} MEK cells co-transfected with FLAG-MEF2C (red) and HDAC5-GFP (green). These cells were stimulated with fluid flow at 0.2 ml/min for 30 min (no flow controls are also included). Then, the cells were fixed, stained and imaged using confocal microscopy. Scale bar: 16 μm. FIG. 2B shows time-lapse images of HDAC5-GFP translocation stimulated by fluid flow. Time (minutes) after flow initiation is indicated on each panel. The graph shows quantification of fluorescence ratio (nuclear/cytosolic) over time. Scale bar: 16 μm. FIG. 2C shows the quantification of HDAC5-GFP distribution in the cytosol or nucleus in Pkd1^{+/+} and Pkd1^{-/-} cells with or without fluid flow stimulation, in the absence or presence of GdCl₃, Rab8^{T22N} transfection (blocking cilia assembly), and HDAC5^{S250/S489a} mutations as indicated. Percentages of cells (over total) with HDAC5-GFP in the nucleus or cytosol or in both compartments are shown. FIG. 2D shows quantification of HDAC5-GFP distribution in the cytosol or nucleus in cells treated with DMSO (solvent control), 100 nM PMA, 10 μM GÖ 6983 in the absence or presence of fluid flow as indicated. For both FIGS. 2C and 2D, more than 600 cells were counted for each experimental condition. Shown are average and SEM from three independent experiments.

FIG. 3 shows that missing in metastasis (MIM) is a transcriptional target of MEF2C and HDAC5. FIG. 3A shows that a chromatin immunoprecipitation (ChIP) experiment demonstrating that MEF2C and HDAC5 bind to the promoter region of MIM. FLAG-MEF2C or FLAG-HDAC5 were transfected into wild-type MEK cells, which were either resting (−flow) or stimulated with fluid flow for 0.5 hour (+flow), and an anti-FLAG antibody was used for ChIP. PCR was performed using primers in the MIM promoter region as explained in Example 1 below. Input is DNA template before ChIP. Ctrl is ChIP using MEK cells transfected with the empty FLAG vector. In two independent experiments each with three independent cultures for flow and non-flow conditions, binding of FLAG-HDAC5 appeared to be reduced in flow-stimulated samples compared to that in the non-flow samples, however, this difference could not be quantified as ChIP is non-quantitative due to the PCR amplification step. FIG. 3B shows that MEF2C is important for normal MIM expression in MEK cells. RNAi knock-down of MEF2C in Pkd1^{+/+} MEK cells led to reduced MIM expression, quantified by qPCR. The results shown are averages of three experiments. Ctrl: cells transfected with control siRNA. Error bars indicate±s.d. FIG. 3C shows the reconstitution of MIM reporter expression in Pkd1^{-/-} MEK cells. Luciferase activity averaged from three experiments are shown for Pkd1^{-/-} cells tranfected with either empty vector (Ctrl), MEF2C alone, GATA6 alone, or MEF2C and GATA6 together. ** indicates P<0.05 compared with control. Error bars indicate standard deviation (s.d.). FIG. 3D shows that HDAC5 negatively regulates MIM expression in MEK cells. Immunoblot and quantification show that HDAC5^{S250/489A}-GFP overexpression reduced MIM expression in Pkd1^{+/+} MEK cells.

FIG. 4 shows that kidney specific knock-out of Mef2C resulted in epithelial tubule dilations/cysts. Kidneys from 5-6 month old Mef2C^{Ioxp/Ioxp} SgIt2-Cre mice and their congenic wild-type controls were fixed in 4% formaldehyde, sectioned (5 μm thick), and stained with hematoxylin and eosin (FIGS. 4A-4G), tubule markers (FIG. 4H) or Ki67 (FIG. 4I). FIG. 4A shows a representative section of wild-type kidney. FIG. 4B shows a representative kidney section from a Mef2C^{Ioxp/Ioxp} SgIt2-Cre mouse. FIG. 4C shows a magnified region corresponding to the box in FIG. 4A. FIG. 4D shows a magnified region corresponding to the box in FIG. 4B, which reveals a broad distribution of dilated tubules (arrows). FIG. 4E shows a 40× magnified cortex region in wild-type mouse kidney, showing normal epithelial tubules. FIG. 4F shows a 40× magnified cortex region of a Mef2C^{Ioxp/Ioxp} SgIt2-Cre kidney, revealing dilated tubules and small cysts with flat lining cells (arrows). FIG. 4G shows glomerular cysts (arrows) in Mef2C^{Ioxp/IoxP} SgIt2-Cre kidney. FIG. 4H shows staining of wild-type and Mef2C^{Ioxp/IxoP} SgIt2-Cre kidney sections against various tubule markers (green): Na⁺/K⁺ ATPase a-1 (which indicates the location of distal tubule), LTA (which indicates the location of proximal tubule) and DBA (which indicates the location of collecting duct). DAPI stain reveals the location of nuclei (blue). Arrows point to dilated tubules/cysts. FIG. 4I shows representative Ki67 staining of wild-type and Mef2C^{Ioxp/Ioxp} SgIt2-Cre kidney sections, showing increased cell proliferation in the mutant kidney section. Scale bars: FIGS. 4A and 4B, 1000 μm; FIGS. 4C and 4D, 200 μm; FIGS. 4E, 4F, 4G, and 4I, 50 μm; FIG. 4H, 100 μm.

FIG. 5 shows that MIM is a transcriptional target of MEF2C and is required for normal renal epithelial organization. FIG. 5A shows the results of a ChIP experiment, which demonstrates that MEF2C binds to the promoter region of MIM that contains a MEF2C-binding site. Flag-MEF2C was transfected into wild-type MEK cells, which were stimulated with or without fluid flow, and an anti-flag antibody was used for ChIP. PCR was performed using primers that flank the predicted MEF2C binding sites. Input, DNA template before ChIP. MEK cells transfected with empty flag vector were used as control (Ctrl). FIG. 5B shows that MEF2C is important for normal MIM expression in MEK cells. RNAi knock-down of MEF2C in Pkd1^{+/+} MEK cells led to reduced MIM expression, quantified by qPCR. The results shown are averages of three experiments. Cells transfected with control siRNA were used as control (Ctrl). Error bars indicate±standard deviation (s.d.) FIG. 5C shows reconstitution of MIM reporter expression in Pkd1^{-/-} MEK cells. Luciferase activity averaged from three experiments are shown for Pkd1^{-/-} cells transfected with either empty vector (Ctrl), MEF2C alone, GATA6 alone, or MEF2C and GATA6 together. Double asterisks (**) indicate P<0.05 compared with control. Error bars indicate±s.d. FIGS. 5D and 5E show representative histology sections of wild-type (FIG. 5D) and MIM^{-/-} (FIG. 5E) kidneys from 5 month old mice. FIG. 5F shows a magnified image of the boxed region in FIG. 5E. FIG. 5G shows a magnified image of cyst lining cells in the boxed region in FIG. 5F. FIGS. 5H and 5I show representative Ki67 staining of wild-type (FIG. 5H) and MIM^{-/-} kidney sections (FIG. 5I), indicating increased cell proliferation in the mutant kidney section. Scale bars: FIGS. 5D and E, 1000 μm; FIGS. 5F-5I, 50 μm.

FIG. 6 shows the effects of Hdac5 genetic or chemical inactivation on cyst formation in Pkd2^{-/-} embryonic kidneys. FIGS. 6A-6E show representative embryonic kidney sections from E18.5 embryos of different genotypes. FIG. 6A shows a kidney section from a Pkd2^{+/+} Hdac5^{+/+} embryo, FIG. 6B shows a kidney section from a Pkd2^{+/+} Hdac5 embryo; FIG. 6C shows a kidney section from a Pkd2^{-/-} Hdac5⁻⁻ embryo; FIG. 6D shows a kidney section from a Pkd2^{-/-} Hdac5^{+/-} embryo; and FIG. 6E shows a kidney section from a Pkd2^{-/-} Hdac5^{+/+} embryo. FIGS. 6F-6H show representative histology sections of E18.5 Pkd2^{+/+} and Pkd2^{-/-} embryonic kidneys from pregnant mothers injected (from 10.5 dpc to 18.5 dpc) with TSA or control solvent (DMSO). FIG. 6F, Pkd2^{+/+} from TSA-treated mother; FIG. 6G, Pkd2^{-/-} from TSA-treated mother; FIG. 6H, Pkd2^{-/-} from DMSO-treated mother. FIG. 6I shows quantification of the percentage of cystic areas over total kidney section areas of different genotypes or treatment as indicated. The middle section of each kidney was quantified for all mice under each condition. Shown are mean and SEM of all sections quantified for each condition. Asterisks, P<0.05 compared with the control to the left. FIGS. 6J, 6K and 6L show magnified images of the box region in FIGS. 6F, 6G, and 6H respectively. Lining cells around a normal tubule (FIG. 6J), small cyst (FIG. 6K) and large cyst (FIG. 6L) are indicated by arrows. FIG. 6M shows immonublots demonstrating reduced MIM and MEF2C expression in 18.5 dpc Pkd2^{-/-} embryonic kidneys. FIG. 6N shows that TSA stimulates MIM and MEF2C expression in Pkd2^{-/-} embryonic kidneys. Scale bars: 6A-6H, 200 μm; 6J-6L, 50 μm.

FIG. 7 shows a schematic diagram depicting a pathway that connects polycystins and calcium signaling to HDAC5 and MEF2C-based transcription activation in the suppression of epithelial cyst formation. Fluid flow through lumens of renal tubules activates the polycystin-1 and 2 receptor-calcium channel complex. Downstream of the intracellular calcium rise, active PKC directly or indirectly causes phosphorylation of HDAC5 and its export from the nucleus, enabling activation of MEF2C-regulated transcripts, which are likely to encode proteins that maintain the normal differentiated state of renal epithelial cells. The observed increase of HDAC5 and MEF2C transcript levels in the microarray analysis may be explained by feedback loops, depicted with dotted lines.

FIG. 8 shows the confirmation of HDAC5, MIM and PKD1 antibody specificities. FIG. 8A is an immunoblot analysis of protein extracts from Hdac^{+/+} and Hdac5^{-/-} adult mouse kidneys with the anti-HDAC5 antibody. FIG. 8B is an immunoblot analysis of protein extracts from MIM^{+/+} and MIM^{-/-} adult mouse kidneys with the anti-MIM antibody. FIG. 8C is an immunoblot analysis of protein extracts from Pkd1^{-/-} and Pkd1^{-/-} MEK cells with the antibody against PC-1.

FIG. 9 shows the flow experiment setup. FIG. 9A shows that fluid flow was applied using a peristaltic pump at a pump rate of 0.2 ml/min across a 3.5 cm (diameter) dish or 6-well plate. FIG. 9B shows a diagram of computed flow field distribution based on the experiment setup. The smaller circle represents the coverslip on which cells were grown for imaging experiments. For all other experiments cells were grown directly on the culture dish. FIG. 9C shows that the cells were grown to confluency and then differentiated for 1-2 days. DAPI (blue), phalloidin (red) and acetylated tubulin antibody (green) stainings show nuclei (blue), cell boundaries (F-actin, red) and cilia (green), respectively. Scale bars: 16 μm.

FIG. 10 shows the quantification of intra-cellular calcium rise in response to fluid flow using the genetically coded calcium sensor GCAMP2. GCAMP2 transfected MEK cells were stimulated with fluid flow at 20s after start of imaging (arrow in top panel). The first peak of GCAMP2 fluorescence occurs 1-2 min after the start of fluid flow. The bottom panel shows a cell imaged long enough to demonstrate 3-4 calcium transients.

FIG. 11 shows the results of an microarray analysis to identify genes induced by fluid flow in a PC-1 dependent manner. FIG. 11A is a heat map showing genes that were induced by flow in pkd1^{+/+} but not in pkd1^{-/-} MEK cells. FIG. 11B shows functional categorization of the genes induced by fluid flow in a PC1-dependent manner, as revealed by expression microarray analysis of Pkd1^{+/+} and Pkd1^{-/-} MEK cells.

FIG. 12 shows the quantification of HDAC5 phosphorylation against total HDAC5. FIGS. 12A and 12B show immunoblots of total HDAC5 in response to fluid flow for 4 hours in Pkd1^{+/+} (FIG. 12A) and Pkd1^{-/-} (FIG. 12B). FIG. 12C shows quantification of HDAC5 phosphorylation (top band) against total HDAC5 over time.

FIG. 13 shows the knockdown of PC-1 in MEK cells using Pkd1 siRNA lentivirus. Pkd1 siRNA reduced PC1 expression by 85% compared to PC-1 in control virus transduced cells.

FIG. 14 shows HDAC5, MEF2C, GATA6 and MIM expression quantification. FIG. 14A shows that MIM expression was increased in Pkd1^{+/+} MEK cells after fluid flow with microarray and Northern quantification. FIGS. 14B and 14C show that compared with Pkd1^{+/+} MEK cells, the expression of HDAC5, MEF2C, GATA6 and MIM in Pkd1^{-/-} cells was reduced dramatically as demonstrated by microarray (FIG. 14B) and qPCR (FIG. 14C) quantification.

FIG. 15 shows Sglt2-Cre expression in kidneys of new-born mice from the cross of Sglt2-Cre with the Gt(ROSA)26Sortm1Sor reporter (Jackson laboratory). FIG. 15A shows frozen-section X-Gal staining of a SgIt2-Cre/R26R kidney. FIG. 15B shows LTA chromogen staining on a frozen-section X-Gal staining slide. FIG. 15C shows Na⁺/K⁺ ATPase a-1 chromogen staining on a frozen-section X-Gal staining slide. FIG. 15D shows DBA chromogen staining on a frozen-section X-Gal staining slide. Scale bars: 50 μm.

FIG. 16 shows MIM knock-out mouse generation and MIM expression pattern in mouse kidney. FIG. 16A is a diagram showing genetrap disruption of the MIM gene. It was confirmed by RACE that the exon 1 of the trapped MIM gene abuts the splice acceptor sequence/Engrailed-2 (En2) exon after transcription and intron splicing. FIGS. 16B-E show MIM expression in embryonic and adult kidneys of MIM^{+/-} mice demonstrated using X-Gal staining. Whole-mount X-Gal staining of E16.5 MIM^{+/-} embryonic kidneys (FIGS. 16B-16C) and frozen-section X-Gal staining of MIM^{+/-} and MIM^{+/+} adult kidneys (FIGS. 16D-16E). FIGS. 16C and 16E are images at a higher resolution than that in FIGS. 16B and 16D. These figures show that MIM is expressed in collecting ducts (CD), tubules (T) and glomeruli (G) in E16.5 kidney (as shown in FIGS. 16B and 16C), and in glomeruli and tubules in adult kidney (FIGS. 16D and 16E). Scale bars: B,C and E, 50 μm; D, 1000 μm.

FIGS. 17A and 17B show that HDAC inhibitors inhibit cAMP-induced cysts in mouse embryonic kidney organ cultures.

FIG. 18 shows that GFP-Rab8^{T22N} blocked cilia formation in transfected MEK cells. The location of GFP-Rab8^{T22N} is shown in green, the location of acetylated tubulin (cilia marker) is shown in red, and the location of the DAPI-stained nuclei are shown in blue. Note the presence of cilia in surrounding non-transfected cells. Scale bar: 16 μm.

FIG. 19A shows an immunoblot analysis demonstrating decreased expression of HDAC5, MEF2C and MIM in human ADPKD cyst lining cells compared to normal human kidney epithelial cells (WT). FIG. 19B shows a quantitative analysis of the immunoblot shown in FIG. 19A. Actin was used as the standard for normalization. FIG. 19C is an immunoblot analysis using a phospho-specific antibody against HDAC5 demonstrating fluid flow-stimulated phosphorylation of HDAC5 in normal human kidney epithelial cells (WT) but not in ADPKD cyst lining cells. FIG. 19D shows the amount of HDAC5 under control and flow conditions. The quantifications are based on the immunoblot shown in FIG. 9C, after normalization using actin as the standard.

FIG. 20 shows a quantitative analysis of the immunoblot shown in FIG. 1D. Actin was used as the standard for normalization.

FIG. 21 shows that MIM^{-/-} mice are viable but exhibit polycystic kidneys by 5 months.

FIG. 22 shows that animals treated with SAHA had significantly lower blood urea nitrogen (BUN) levels, compared to vehicle treated animals, indicating improved kidney function. 8 week old PKD^{ws25/-} mice (n=15 per group) were treated with 100 mg/kg vorinostat (SAHA) or vehicle (Veh). Animals were dosed daily by i.p. for 5 days followed by 2 days off, for a total of 4 weeks. Both treatments were well tolerated, one animal was lost from the vorinostat group. At the end of the study, animals were sacrificed and blood taken for analysis of BUN levels.

FIG. 23 shows that SAHA treatment of pkd2^{ws25/-} adult mice drastically improved renal tubular epithelial morphology. In each column, top to bottom show images of a representative SAHA-treated (FIGS. 23A, 23C, and 23E) or control (Ctrl, FIGS. 23B, 23D, and 23F) mouse kidney with increasing magnification. Small cysts or tubule dilations are widely observed in the control images whereas SAHA-treated images show well organized, nearly normal epithelial morphology.

### DETAILED DESCRIPTION

One embodiment of the present invention is a method of treating or ameliorating an effect of a polycystic disease. This method comprises administering to a patient in need thereof an amount of a modulator of a histone deacetylase (HDAC) pathway, which amount is sufficient to treat or ameliorate an effect of a polycystic disease.

“Treatment” or “treating,” as used herein means preventing, suppressing, repressing, or completely eliminating a disease, such as, e.g., a polycystic disease. Preventing a disease involves administering a composition of the present invention to a patient, such as a mammal, preferably a human, prior to onset of the disease. Suppressing a disease involves administering a composition of the present invention to a patient after induction of a disease but before its clinical appearance. Repressing a disease involves administering a composition of the present invention to a patient after clinical appearance of the disease. “Ameliorating” an effect of a disease means that at least one symptom of the disease is eliminated or decreased.

Non-limiting examples of a polycystic disease according to the present invention include polycystic kidney disease (PKD), polycystic liver disease (PLD), polycystic ovary syndrome (PCOS), pancreatic cysts, cancer, or combinations thereof. Preferably, the polycystic disease is autosomal dominant polycystic kidney disease (ADPKD) or autosomal recessive polycystic kidney disease (ARPKD).

“Cancer” as used herein may mean the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma) esophagus, stomach, gall bladder, cervix, thyroid, renal, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoeietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; and other tumors including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

In the present invention, a “modulator of an HDAC pathway” means any agent that regulates the activity of any member of the HDAC pathway, which results in, e.g., decreased expression of HDAC, increased nuclear export of HDAC, increased retention of HDAC in the cytoplasm, or activation of transcription factors such as MEF2. A modulator of the HDAC pathway may act upstream or downstream of HDAC in the HDAC pathway.

Representative, non-limiting examples of members of an HDAC pathway according to the present invention include heterotrimeric G proteins, phospholipase C, protein kinase C, protein kinase D, inositol 1, 4, 5 triphosphate receptor (IP3R), calcium calmodulin kinase II, salt inducible kinase 1 (Sik1), 14-3-3 proteins, MEF2, GATA, and MIM.

For example, in the HDAC5 pathway, heterotrimeric G-proteins, which are composed of an alpha subunit, a beta subunit, and a gamma subunit, are activated by receptors that are associated with them. Upon activation, the G-proteins activate phospholipase C, which cleaves phosphatidylinositol 4,5-bisphosphate (PIP₂) into diacyl glycerol (DAG) and inositol 1,4,5-triphosphate (IP₃). IP₃ actives receptors, such as IP3R. One of the effects of activating IP3R is an increase in calcium level, which activates calcium calmodulin kinase II. Calcium calmodulin kinase II is one of the kinases that phosphorylate HDAC5 at positions which result in dissociation of HDAC5 from MEF2C and translocation of HDAC5 from the nucleus to the cytosol. Other kinases that are able to effect translocation of HDAC5 include Sik1 and protein kinase D. Protein kinase D may be activated by protein kinase C, and DAG can activate both protein kinase C and protein kinase D. Once HDAC5 is in the cytosol, it binds to 14-3-3 proteins. The dissociation and translocation of HDAC5 allow for MEF2C and its co-factors, such as GATA6, to transcribe target genes such as MIM.

In one aspect of this embodiment, the modulator modulates a class II HDAC. As used herein, “modulating”, “modulation” and like terms mean altering the function of or expression level of a protein, e.g., a protein in the HDAC pathway, including but not limited to lowering or increasing the expression level of a protein (either at the transcription stage or the translation stage), altering the sequence of such a protein (by, e.g., mutation, pre-translational or post-translational modification or otherwise), or inhibiting or activating such a protein (by, e.g., binding, phosphorylation, glycosylation, translocation or otherwise). Such modulation may be achieved genetically or pharmacologically.

A “Class II HDAC” means the phylogenetic class of HDACs that share domains with similarity to the yeast deacetylase HDA1. Representative non-limiting members of the HDAC class II family include HDAC4, 5, 6, 7, 9, and 10. Preferably, the class II HDAC is HDAC5. In another preferred aspect of this embodiment, the modulator inhibits HDAC5. As used herein, “inhibit” and “inhibiting” and like terms, when used with respect to HDAC5, means a decrease of the function of HDAC5 as a repressor of gene transcription. Non-limiting examples of how the function of HDAC5 may be decreased include decreasing expression of HDAC5, increasing nuclear export of HDAC5, and increasing retention of HDAC5 in the cytoplasm.

In a further aspect of this embodiment, the modulator of the HDAC pathway is an HDAC inhibitor (HDACi). Preferably, the HDACi is selected from the group consisting of nucleic acids, polypeptides, polysaccharides, small organic or inorganic molecules, and combinations thereof.

Non-limiting examples of HDACi according to the present invention include Entinostat (Bayer A G, Leverkusen, Germany), KD-5170 (Kalypsys, San Diego, Calif.), KD-5150 (Kalypsys, San Diego, Calif.), KLYP-278 (Kalypsys, San Diego, Calif.), KLYP-298 (Kalypsys, San Diego, Calif.), KLYP-319 (Kalypsys, San Diego, Calif.), KLYP-722 (Kalypsys, San Diego, Calif.), CG-200745 (CrystalGenomics, Inc., Seoul, South Korea), Avugane (TopoTarget AS, København, Denmark), SB-939 (S*BIO, Singapore), ARQ-700RP (ArQule, Woburn, Mass.), KA-001 (Karus Therapeutics, Chilworth, Hampshire, United Kingdom), MG-3290 (MethylGene, Montreal, Quebec, Canada), PXD-118490 (LEO-80140) (TopoTarget AS, København, Denmark), CHR-3996 (Chroma Therapeutics, Abingdon, Oxon, United Kingdom), AR-42 (Arno Therapeutics, Parsippany, N.J.), RG-2833 (RepliGen, Waltham, Mass.), DAC-60 (Genextra, Milan, Italy), SB-1304 (S*BIO, Singapore), SB-1354 (S*BIO, Singapore), 4SC-201 (4SC AG, Planegg-Martinsried, Germany), 4SC-202 (4SC AG, Planegg-Martinsried, Germany), NBM-HD-1 (NatureWise, Biotech & Medicals, Taipei, Taiwan), CU-903 (Curis, Cambridge, Mass.), MG-2856 (MethylGene, Montreal, Quebec, Canada), MG-4230 (MethylGene, Montreal, Quebec, Canada), MG-4915 (MethylGene, Montreal, Quebec, Canada), MG-5026 (MethylGene, Montreal, Quebec, Canada), pharmaceutically acceptable salts thereof, or combinations thereof.

In one aspect of this embodiment, the HDACi is selected from the group consisting of hydroxamic acids, short chain fatty acids, cyclic tetrapeptides/epoxides, benzamides, electrophilic ketone derivatives, and combinations thereof.

As used herein, a “hydroxamic acid” means a chemical compound sharing the same functional group represented by the formula —CO—NH—OH. Preferred hydroxamic acids according to the present invention also contain a phenyl group, and such hydroxamic acids may be represented by the following general structure:

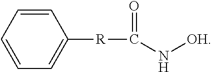

Non-limiting examples of hydroxamic acids according to the present invention include trichostatin A (TSA), suberoylanilide hydroxamic acid (SAHA), pyroxamide(suberoyl-3-aminopyridineamide hydroxamic acid), azelaic-1-hydroxamate-9-anilide (AAHA), compound 13, compound 14, CRA-024781 (Pharmacyclics, Sunnyvale, Calif.), bombesin-2 (BB2) receptor antagonist, JNJ-16241199 (Johnson & Johnson, Langhorne, Pa.), Oxamflatin ((2E)-5-[3-[(phenylsulfonyl)amino]phenyl]-pent-2-en-4-ynohydroxamic acid), CG-1521 (Errant Gene Therapeutics, LLC, Chicago, Ill.), CG-1255 (Errant Gene Therapeutics, LLC, Chicago, Ill.), SK-7068 (In2Gen/SK Chemical Co., Suweon, Korea), SK-7041 (In2Gen/SK Chemical Co., Suweon, Korea), m-carboxycinnamic acid bis-hydroxamide (CBHA), Scriptaid (N-Hydroxy-1,3-dioxo-1H-benz[de]isoquinoline-2(3H)-hexan amide), compound 48, compound 49, compound 50, compound 51, SB-623 (Merrion Research I Limited, National Digital Park, Ireland), SB-639 (Merrion Research I Limited, National Digital Park, Ireland), SB-624 (Merrion Research I Limited, National Digital Park, Ireland), Panobinostat (LBH-589) (Novartis, Basel, Switzerland), NVP-LAQ824 (Novartis, Basel, Switzerland), compound 70, pharmaceutically acceptable salts thereof, and combinations thereof.

Pyroxamide may be synthesized according to Butler et al., “Inhibition of Transformed Cell Growth and Induction of Cellular Differentiation by Pyroxamide, an Inhibitor of Histone Deacetylase,” Clinical Cancer Research, 7:962-970 (2001). Synthesis of AAHA has been described in Qiu et al., “Anti-tumour activity in vitro and in vivo of selective differentiating agents containing hydroxamate,” Br. J. Cancer 80:12521258 (1999). BB2 receptor antagonists are described in, for example, Ashwood et al., “The first high affinity non-peptide gastrin-releasing peptide (BB2) receptor antagonist,” Bioorg Med Chem Lett, 8(18): 2589-94 (1998). Oxamflatin and Scriptaid are commercially available from, e.g., Enzo Life Sciences, Inc. (Plymouth Meeting, Pa.). CBHA is commercially available from, e.g., Cayman Chemical Co. (Ann Arbor, Mich.). The structures of compounds 13, 14, 48, 49, 50, 51, and 70 are as follows:

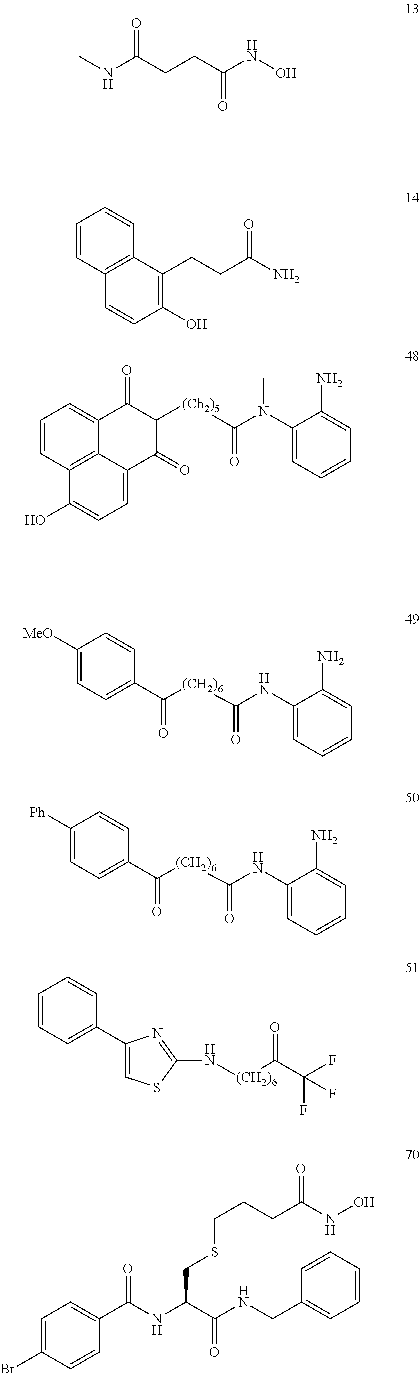

These compounds were disclosed in Balakin et al., “Histone Deacetylase Inhibitors in Cancer Therapy: Latest Developments, Trends and Medicinal Chemistry Perspective,” Anti-Cancer Agents in Medicinal Chemistry, 7:576-592 (2007).

As used herein, a “short chain fatty acid” means a chemical compound containing a carboxylic acid and containing an aliphatic chain of less than 20 carbons, preferably eight or fewer carbons, for example, 2-6 carbons. A “carboxylic acid” means a chemical compound containing a functional group represented by the formula —COOH. An “aliphatic chain” means a functional group containing only carbon and hydrogen atoms that is not an aromatic. Aromatic functional groups (and other functional groups), however, may be present in addition to the aliphatic chain. The aliphatic chain may be branched or unbranched, and it may be saturated or unsaturated. Short chain fatty acids may be represented by the following general structure:

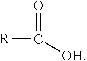

Non-limiting examples of short chain fatty acids according to the present invention include butyrate, phenylbutyrate, valporic acid (VPA), Pivanex™ (Titan Pharmaceuticals, Inc.), AN-1 (Titan Pharmaceuticals, Inc.), tributyrin, compound G1, pivaloyloxymethyl butyrate (AN-9, Titan Pharmaceuticals, Inc.), hyaluronic acid butyric acid ester (HA-But), pharmaceutically acceptable salts thereof, and combinations thereof.

Butyrate and phenylbutyrate are commercially available from, e.g., Enzo Life Sciences, Inc. Tributyrin is commercially available from, e.g., Colonial Scientific (Richmond, Va.). HA-But may be obtained by the esterification of butyric acid (BA), the smallest HDAC inhibitor, with hyaluronic acid (HA) and may be prepared as disclosed in Speranza et al., “Hyaluronic acid butyric esters in cancer therapy” Anticancer Drugs. 16(4):373-9. (2005). The structure of compound G1 is as follows:

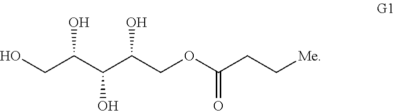

Compound G1 was disclosed in Balakin et al., “Histone Deacetylase Inhibitors in Cancer Therapy Latest Developments, Trends and Medicinal Chemistry Perspective” Anti-Cancer Agents in Medicinal Chemistry, 7:576-592 (2007).

As used herein, “a cyclic tetrapeptide” means a cyclic compound containing four amino acids, which may be natural, synthetic, or a modification, or a combination of natural and synthetic amino acids, joined by peptide bonds. Cyclic tetrapeptides may be represented by the following general structure:

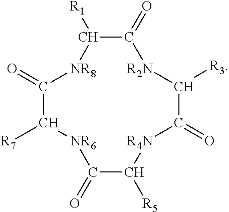

An “epoxide” means a compound containing a cyclic ether with three ring atoms, or a compound which may be prepared from such a cyclic ether. A compound containing a cyclic ether with three ring atoms may be represented by the following general structure:

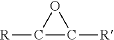

Non-limiting examples of a cyclic tetrapeptide/epoxide according to the present invention include Apicidine (Merck & Co., Inc., Whitehouse Station, N.J.), Trapoxin-A (cyclo((S)-phenylalanyl-(S)-phenylalanyl-(R)-pipecolinyl-(2S,9S)-2-amino-8-oxo-9,10-epoxydecanoyl), Trapoxin-B (cyclo[(S)-phenylalanyl-(S)-phenylalanyl-(R)-prolyl-2-amino-8-oxo-9,10-epoxydecanoyl-]), cyclic hydroxamic acid-containing peptide 1 (CHAP-1), CHAP-31, CHAP-15, chlamidocin, HC-toxin, WF-27082B (Fujisawa Pharmaceutical Company, Ltd., Osaka, Japan), Romidepsin (Gloucester Pharmaceuticals, Cambridge, Mass.), Spiruchostatin A, Depudesin, compound D1, Triacetylshikimic acid, Cyclostellettamine FFF1, Cyclostellettamine FFF2, Cyclostellettamine FFF3, Cyclostellettamine FFF4, pharmaceutically acceptable salts thereof, and combinations thereof.

Trapoxins A and B may be isolated, e.g., from the culture broth of Helicoma ambiens RF-1023 as described by Itazaki et al., “Isolation and structural elucidation of new cyclotetrapeptides, trapoxins A and B, having detransformation activities as antitumor agents,” J. Antibiot (Tokyo), 43(12):1524-32 (1990). CHAP-1 and CHAP-15 may be synthesized, e.g., as disclosed in Furumai et al., “Potent histone deacetylase inhibitors built from trichostatin A and cyclic tetrapeptide antibiotics including trapoxin,” Proc. Natl. Acad. Sci., 98:87-92 (2001). CHAP-31 may be synthesized, e.g., as disclosed in Komatsu et al., “Cyclic Hydroxamic-acid-containing Peptide 31, a Potent Synthetic Histone Deacetylase Inhibitor with Antitumor Activity.” HC-toxin and Depudesin are commercially available from, e.g., Enzo Life Sciences, Inc. Spiruchostatin A may be synthesized, e.g., according to Yurek-George et al., “Total synthesis of spiruchostatin A, a potent histone deacetylase inhibitor” Journal of the American Chemical Society, 126(4): 1030-1031 (2004). The cyclostellettamines may be isolated as described in Fusetani et al., “Three new cyclostellettamines, which inhibit histone deacetylase, from a marine sponge of the genus Xestospongia.” Bioorg Med Chem. Lett. 14(10):2617-20 (2004). The structure of compound D1 is as follows:

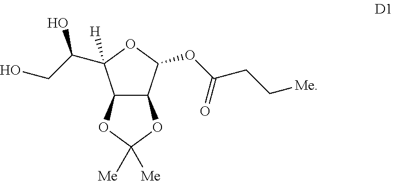

Compound D1 and triacetylshikimic acid are disclosed, e.g., in Balakin et al., “Histone Deacetylase Inhibitors in Cancer Therapy: Latest Developments, Trends and Medicinal Chemistry Perspective” Anti-Cancer Agents in Medicinal Chemistry, 7:576-592 (2007).

As used herein, a “benzamide” means a compound containing the following functional group:

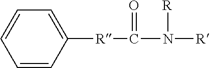

Preferred benzamides according to the present invention may be represented by the following general structure:

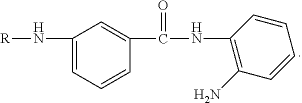

Non-limiting examples of benzamides include MS-27-275 (Schering AG, Germany), Tacedinaline (N-acetyldinaline), compound 27, ITF-2357 (Italfarmaco, Cinisello Balsamo, Italy), compound 29, compound 30, N-hydroxy-4-(3-methyl-2-phenyl-butyrylamino)benzamide (HDAC-42), MGCD-0103 (MethylGene Inc., Montreal, Quebec, Canada), PX-117794 (TopoTarget AS, København, Denmark), compound 37, Belinostat (TopoTarget AS, København, Denmark), Compound 39, sulfonamide hydroxamic acid, pharmaceutically acceptable salts thereof, and combinations thereof.

Tacedinaline is commercially available from, e.g., Medical Isotopes, Inc. (Pelham, N.H.). HDAC-42 may be synthesized, e.g., according to Lu et al., “Structure-based optimization of phenylbutyrate-derived histone deacetylase inhibitors” J Med. Chem. 48(17):5530-5 (2005). The structures of compounds 27, 29, 30, 37, and 39 are as follows:

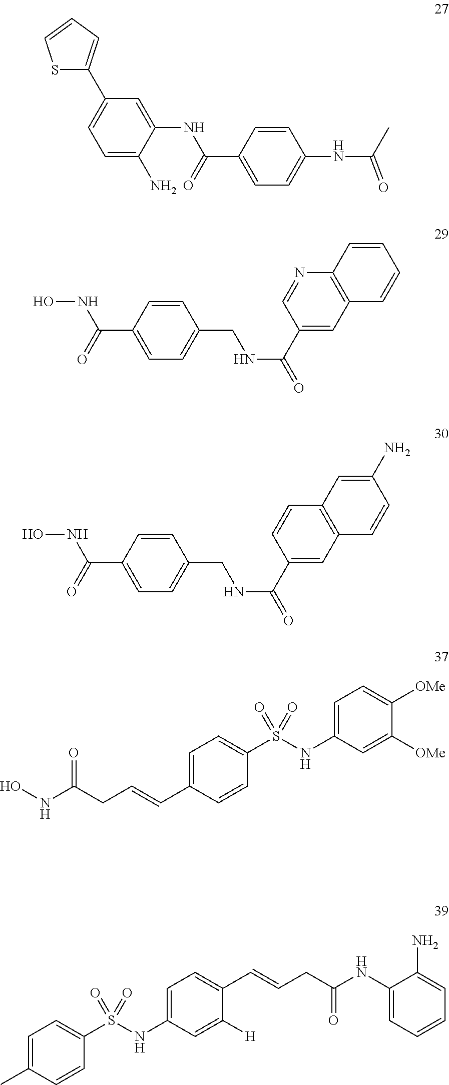

These compounds, as well as sulfonamide hydroxamic acid, are disclosed in Balakin et al., “Histone Deacetylase Inhibitors in Cancer Therapy: Latest Developments, Trends and Medicinal Chemistry Perspective” Anti-Cancer Agents in Medicinal Chemistry, 7:576-592 (2007).

As used herein, an “electrophilic ketone derivative” means a compound containing a ketone group that may be an acceptor of electron pairs in a chemical reaction. Non-limiting examples of electrophilic ketone derivatives include, e.g., a trifluoromethyl ketone or an alpha-keto amide. As used herein, a “trifluoromethyl ketone” means a compound containing a functional group represented by the formula —COCF₃. An alpha-keto amide means a compound containing a functional group represented by the following structure:

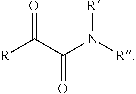

Preferably, the HDACi is selected from the group consisting of TSA, SAHA, VPA, and combinations thereof.

In anther aspect of this embodiment, the modulator regulates the activity of a myocyte enhancer factor 2 (MEF2) protein, an heterotrimeric G protein, a phospholipase C(PLC), a protein kinase C(PKC), a protein kinase D (PKD), an inositol 1, 4, 5 triphosphate receptor (IP3R), a calcium calmodulin kinase II (CaMKII), a salt inducible kinase 1 (Sik1), or a 14-3-3 polypeptide. Preferably, the MEF2 protein is a MEF2C protein.

In another preferred embodiment, the modulator is a MIM activator, a GATA activator, a MEF2 activator, an heterotrimeric G protein activator, a PLC activator, a PKC activator, a PKD activator, an IP3R activator, a CaMKII activator, a Sik1 activator, or a 14-3-3 activator. Preferably, the GATA activator is a GATA6 activator.

An “activator” of a polypeptide according to the present invention increases the function of the polypeptide by, e.g., increasing the expression of the polypeptide or by binding to the polypeptide.

Non-limiting examples of an heterotrimeric G protein activator include mastoparan, fluoroaluminate (AlF4⁻), guanosine 5′—O—(3-thiotriphosphate), G-protein bg (beta gamma) binding peptide mSIRK, MAS 7, Pasteurella multocida toxin, and combinations thereof. Fluoroaluminate may be prepared, e.g., according to Jeschke et al., “Fluoroaluminate Induces Activation and Association of Src and Pyk2 Tyrosine Kinases in Osteoblastic MC3T3-E1 Cells” J. Biol. Chem., 273 (18):11354-11361 (1998). Mastoparan, guanosine 5′-O-(3-thiotriphosphate), G-protein bg (beta gamma) binding peptide mSIRK, MAS 7, and Pasteurella multocida toxin are commercially available from, e.g., Merck Chemical Ltd. (Nottingham, United Kingdom).

A non-limiting example of a PLC activator is m-3M3FBS, which is commercially available from, e.g., Sigma (St. Louis, Mo.).

Non-limiting examples of a PKC activator include 12-myristate 13-acetate (PMA), phorbol 12,13-dibutyrate (PDBu), phorbol 12,13-didecanoate (PDD), farnesyl thiotriazole, ingenol 3,20-dibenzoate, (−)-7-octylindolactam V, n-heptyl-5-chloronaphthalene-1-sulfonamide, mezerein, ingenol mebutate (Peplin, Emeryville, Calif.), KAI-1455 (KAI Pharmaceuticals, South San Francisco, Calif.), KAI-9706 (KAI Pharmaceuticals, South San Francisco, Calif.), bryostatin-1 nanosome (Aphios, Woburn, Mass.), bryostatin-1, Sapintoxin A, 8-octyl-benzolactam-V9,1-hexylindolactam-V10, phorbol 12-myristate 13-acetate, cholesterol sulfate, daphnoretin, DiC8, farnesyl thiotriazole, and combinations thereof. PMA, PDBu, PDD, farnesyl thiotriazole, ingenol 3,20-dibenzoate, (−)-7-octylindolactam V, mezerein, bryostatin-1, Sapintoxin A, and farnesyl thiotriazole are commercially available from, e.g., Enzo Life Sciences, Inc. 8-octyl-benzolactam-V9 may be synthesized, e.g., as disclosed by Nakagawa et al., “Design and synthesis of 8-octyl-benzolactam-V9, a selective activator for protein kinase C epsilon and eta” J Med. Chem. 49(9):2681-8 (2006). n-heptyl-5-chloronaphthalene-1-sulfonamide is commercially available from, e.g., Sigma. 1-hexylindolactam-V10 may be made, e.g., as disclosed by Yanagita et al., “Synthesis, Conformational Analysis, and Biological Evaluation of 1-Hexylindolactam-V10 as a Selective Activator for Novel Protein Kinase C Isozymes” J Med. Chem. 51(1):46-56 (2008). Phorbol 12-myristate 13-acetate is commercially available from, e.g., Calbiochem (San Diego, Calif.). Cholesterol sulfate is commercially available from, e.g., Axxora LLC (San Diego, Calif.). Daphnoretin may be isolated according to Ko et al., “Daphnoretin, a new protein kinase C activator isolated from Wikstroemia indica C.A. Mey” Biochem J. 295 (Pt 1):321-7 (1993). DiC8 is commercially available from, e.g., Echelon Biosciences Incorporated (Salt Lake City, Utah).

A non-limiting example of an IP3R activator is adenophostin, which is commercially available from A.G. Scientific, Inc. (San Diego, Calif.).

Another embodiment of the present invention is a method of treating or ameliorating an effect of a polycystic kidney disease (PKD). This method comprises administering to a patient in need thereof an amount of an HDAC inhibitor (HDACi) that is sufficient to treat or ameliorate an effect of PKD.

In one aspect of this embodiment, the HDACi is selected from the group consisting of hydroxamic acids, short chain fatty acids, cyclic tetrapeptides/epoxides, benzamides, electrophilic ketone derivatives, and combinations thereof. Hydroxamic acids, short chain fatty acids, cyclic tetrapeptides/epoxides, benzamides, and electrophilic ketone derivatives are as described above.

In another aspect of this embodiment, the HDACi is selected from the group consisting of Entinostat (Bayer A G, Leverkusen, Germany), KD-5170 (Kalypsys, San Diego, Calif.), KD-5150 (Kalypsys, San Diego, Calif.), KLYP-278 (Kalypsys, San Diego, Calif.), KLYP-298 (Kalypsys, San Diego, Calif.), KLYP-319 (Kalypsys, San Diego, Calif.), KLYP-722 (Kalypsys, San Diego, Calif.), CG-200745 (CrystalGenomics, Inc., Seoul, South Korea), Avugane (TopoTarget AS, København, Denmark), SB-939 (S*BIO, Singapore), ARQ-700RP (ArQule, Woburn, Mass.), KA-001 (Karus Therapeutics, Chilworth, Hampshire, United Kingdom), MG-3290 (MethylGene, Montreal, Quebec, Canada), PXD-118490 (LEO-80140) (TopoTarget AS, København, Denmark), CHR-3996 (Chroma Therapeutics, Abingdon, Oxon, United Kingdom), AR-42 (Arno Therapeutics, Parsippany, N.J.), RG-2833 (RepliGen, Waltham, Mass.), DAC-60 (Genextra, Milan, Italy), SB-1304 (S*BIO, Singapore), SB-1354 (S*BIO, Singapore), 4SC-201 (4SC AG, Planegg-Martinsried, Germany), 4SC-202 (4SC AG, Planegg-Martinsried, Germany), NBM-HD-1 (NatureWise, Biotech & Medicals, Taipei, Taiwan), CU-903 (Curis, Cambridge, Mass.), MG-2856 (MethylGene, Montreal, Quebec, Canada), MG-4230 (MethylGene, Montreal, Quebec, Canada), MG-4915 (MethylGene, Montreal, Quebec, Canada), MG-5026 (MethylGene, Montreal, Quebec, Canada), trichostatin A (TSA), suberoylanilide hydroxamic acid (SAHA), pyroxamide, azelaic-1-hydroxamate-9-anilide (AAHA), compound 13, compound 14, CRA-024781 (Pharmacyclics, Sunnyvale, Calif.), bombesin-2 (BB2) receptor antagonist, JNJ-16241199 (Johnson & Johnson, Langhorne, Pa.), Oxamflatin, CG-1521 (Errant Gene Therapeutics, LLC, Chicago, Ill.), CG-1255 (Errant Gene Therapeutics, LLC, Chicago, Ill.), SK-7068 (In2Gen/SK Chemical Co., Suweon, Korea), SK-7041 (In2Gen/SK Chemical Co., Suweon, Korea), m-carboxycinnamic acid bis-hydroxamide (CBHA), Scriptaid (N-Hydroxy-1,3-dioxo-1H-benz[de]isoquinoline-2(3H)-hexan amide), compound 48, compound 49, compound 50, compound 51, SB-623 (Merrion Research I Limited, National Digital Park, Ireland), SB-639 (Merrion Research I Limited, National Digital Park, Ireland), SB-624 (Merrion Research I Limited, National Digital Park, Ireland), Panobinostat (LBH-589) (Novartis, Basel, Switzerland), NVP-LAQ824 (Novartis, Basel, Switzerland), compound 70, butyrate, phenylbutyrate, valporic acid (VPA), Pivanex™ (Titan Pharmaceuticals, Inc.), AN-1 (Titan Pharmaceuticals, Inc.), Tributyrin, compound G1, Pivaloyloxymethyl butyrate, Apicidine, Trapoxin-A, Trapoxin-B, cyclic hydroxamic acid-containing peptide 1 (CHAP-1), CHAP-31, CHAP-15, chlamidocin, HC-Toxin, WF-27082B (Fujisawa Pharmaceutical Company, Ltd., Osaka, Japan), Romidepsin (Gloucester Pharmaceuticals, Cambridge, Mass.), Spiruchostatin A, Depudesin, compound D1, Triacetylshikimic acid, hyaluronic acid butyric acid ester (HA-But), Cyclostellettamine FFF1, Cyclostellettamine FFF2, Cyclostellettamine FFF3, Cyclostellettamine FFF4, MS-27-275 (Schering A G, Germany), Tacedinaline (N-acetyldinaline), compound 27, ITF-2357 (Italfarmaco, Cinisello Balsamo, Italy), compound 29, compound 30, N-hydroxy-4-(3-methyl-2-phenyl-butyrylamino)benzamide (HDAC-42), MGCD-0103 (MethylGene Inc., Montreal, Quebec, Canada), PX-117794 (TopoTarget AS, København, Denmark), compound 37, Belinostat (TopoTarget AS, København, Denmark), compound 39, sulfonamide hydroxamic acid, trifluoromethyl ketone, an alpha-keto amide, pharmaceutically acceptable salts thereof, and combinations thereof.

In a further aspect of this embodiment, the HDACi inhibits a class II HDAC.

In another aspect of this embodiment, the polycystic kidney disease is autosomal dominant polycystic kidney disease (ADPKD) or autosomal recessive polycystic kidney disease (ARPKD).

A further embodiment of the present invention is a method of treating or ameliorating an effect of a polycystic kidney disease (PKD). This method comprises administering to a patient in need thereof an amount of an HDAC5 inhibitor that is sufficient to treat or ameliorate an effect of PKD. Preferably, the polycystic kidney disease is autosomal dominant polycystic kidney disease (ADPKD) or autosomal recessive polycystic kidney disease (ARPKD). HDAC5 inhibitors are as disclosed herein.

### Additional Definitions

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

a. Nucleic Acid

“Nucleic acid” or “oligonucleotide” or “polynucleotide” used herein mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be a RNA such as a mRNA, tRNA, shRNA, siRNA, Piwi-interacting RNA, pri-miRNA, pre-miRNA, miRNA, or anti-miRNA, as described, e.g., in U.S. patent application Ser. Nos. 11/429,720, 11/384,049, 11/418,870, and 11/429,720 and Published International Application Nos. WO 2005/116250 and WO 2006/126040.

The nucleic acid may also be an aptamer, an intramer, or a spiegelmer. The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), disclosed in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′—NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those disclosed in U.S. Pat. Nos. 5,235,033 and 5,034,506. Nucleic acids containing one or more non-naturally occurring or modified nucleotides are also included within the definition of nucleic acid. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′—OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as disclosed in Krutzfeldt et al., Nature (Oct. 30, 2005), Soutschek et al., Nature 432:173-178 (2004), and U.S. Patent Application Publication No. 20050107325. Modified nucleotides and nucleic acids may also include locked nucleic acids (LNA), as disclosed in U.S. Patent Application Publication No. 20020115080. Additional modified nucleotides and nucleic acids are disclosed in U.S. Patent Application Publication No. 20050182005. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

b. Peptide, Polypeptide, Protein

The terms “peptide,” “polypeptide,” and “protein” are used interchangeably herein. In the present invention, these terms mean a linked sequence of amino acids, which may be natural, synthetic, or a modification, or combination of natural and synthetic. The term includes antibodies, antibody mimetics, domain antibodies, lipocalins, targeted proteases, and polypeptide mimetics. The term also includes vaccines containing a peptide or peptide fragment intended to raise antibodies against the peptide or peptide fragment.

“Antibody” as used herein includes an antibody of classes IgG, IgM, IgA, IgD, or IgE, or fragments or derivatives thereof, including Fab, F(ab′)2, Fd, and single chain antibodies, diabodies, bispecific antibodies, and bifunctional antibodies. The antibody may be a monoclonal antibody, polyclonal antibody, affinity purified antibody, or mixtures thereof which exhibits sufficient binding specificity to a desired epitope or a sequence derived therefrom. The antibody may also be a chimeric antibody. The antibody may be derivatized by the attachment of one or more chemical, peptide, or polypeptide moieties known in the art. The antibody may be conjugated with a chemical moiety. The antibody may be a human or humanized antibody. These and other antibodies are disclosed in U.S. Published Patent Application No. 20070065447.

Other antibody-like molecules are also within the scope of the present invention. Such antibody-like molecules include, e.g., receptor traps (such as entanercept), antibody mimetics (such as adnectins, fibronectin based “addressable” therapeutic binding molecules from, e.g., Compound Therapeutics, Inc.), domain antibodies (the smallest functional fragment of a naturally occurring single-domain antibody (such as, e.g., nanobodies; see, e.g., Cortez-Retamozo et al., Cancer Res. 2004 Apr. 15; 64(8):2853-7)).

Suitable antibody mimetics generally can be used as surrogates for the antibodies and antibody fragments described herein. Such antibody mimetics may be associated with advantageous properties (e.g., they may be water soluble, resistant to proteolysis, and/or be nonimmunogenic). For example, peptides comprising a synthetic beta-loop structure that mimics the second complementarity-determining region (CDR) of monoclonal antibodies have been proposed and generated. See, e.g., Saragovi et al., Science. Aug. 16, 1991; 253(5021):792-5. Peptide antibody mimetics also have been generated by use of peptide mapping to determine “active” antigen recognition residues, molecular modeling, and a molecular dynamics trajectory analysis, so as to design a peptide mimic containing antigen contact residues from multiple CDRs. See, e.g., Cassett et al., Biochem Biophys Res Commun. Jul. 18, 2003; 307(1):198-205. Additional discussion of related principles, methods, etc., that may be applicable in the context of this invention are provided in, e.g., Fassina, Immunomethods. October 1994; 5(2):121-9.

As used herein, “peptide” includes targeted proteases, which are capable of, e.g., substrate-targeted inhibition of post-translational modification such as disclosed in, e.g., U.S. Patent Application Publication No. 20060275823.

In the present invention, “peptide” further includes anticalins. Anticalins can be screened for specific binding to a modulator of the HDAC pathway such as HDAC5, fragments of a modulator of the HDAC pathway, or variants of a modulator of the HDAC pathway such as HDAC5. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art, and can include conservative substitutions (e.g., substitutions that do not alter binding specificity) or substitutions that modestly, moderately, or significantly alter binding specificity.

In general, a polypeptide mimetic (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides, and peptoids. Examples of some peptidomimetics by the broader definition (e.g., where part of a polypeptide is replaced by a structure lacking peptide bonds) are described below. Whether completely or partially non-peptide in character, peptidomimetics according to this invention may provide a spatial arrangement of reactive chemical moieties that closely resembles the three-dimensional arrangement of active groups in a polypeptide. As a result of this similar active-site geometry, the peptidomimetic may exhibit biological effects that are similar to the biological activity of a polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that may be reduced with peptidomimetics.

Polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure, shape or reactivity. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98.

c. Derivative

“Derivative,” as used with respect to a peptide or polypeptide, means a peptide or polypeptide different other than in primary structure (amino acids and amino acid analogs). By way of illustration, derivatives may differ by being glycosylated, one form of post-translational modification. For example, peptides or polypeptides may exhibit glycosylation patterns due to expression in heterologous systems. If at least one biological activity is retained, then these peptides or polypeptides are derivatives according to the invention. Other derivatives may include fusion peptides or fusion polypeptides having a covalently modified N- or C-terminus, PEGylated peptides or polypeptides, peptides or polypeptides associated with lipid moieties, alkylated peptides or polypeptides, peptides or polypeptides linked via an amino acid side-chain functional group to other peptides, polypeptides or chemicals, and additional modifications as would be understood in the art.

d. Fragment

As used herein, the word, “fragment,” when used in the context of a peptide or polypeptide, may mean a peptide of from about 5 to about 150, about 6 to about 100, or about 8 to about 50 amino acids in length.

e. Small Organic or Inorganic Molecules

The phrase “small organic or inorganic molecule” includes any chemical or other moiety, other than polysaccharides, polypeptides, and nucleic acids, that can act to affect biological processes. Small molecules can include any number of therapeutic agents presently known and used, or can be synthesized in a library of such molecules for the purpose of screening for biological function(s). Small molecules are distinguished from macromolecules by size. The small molecules of this invention usually have a molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such as nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, mono-saccharides, di-saccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocyclic compounds, imidizoles, and phenols. An organic compound as used herein also includes nitrated organic compounds and halogenated (e.g., chlorinated) organic compounds. Collections of small molecules, and small molecules identified according to the invention are characterized by techniques such as accelerator mass spectrometry (AMS; see Turteltaub et al., Curr Pharm Des 2000 6:991-1007, Bioanalytical applications of accelerator mass spectrometry for pharmaceutical research; and Enjalbal et al., Mass Spectrom Rev 2000 19:139-61, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

f. Polysaccharides

The term “polysaccharides” means polymeric carbohydrate structures, formed of repeating units (either mono- or di-saccharides) joined together by glycosidic bonds. The units of mono- or di-saccharides may be the same or different. Non-limiting examples of polysaccharides include starch, glycogen, cellulose, and chitin.

### 2. Formulation of a Modulator of an HDAC Pathway

A modulator of an HDAC pathway according to the present invention may be administered to treat a polycystic or other related disease in a number of different formulations. A soluble modulator of an HDAC pathway may be administered in the form of a composition comprising purified protein in conjunction with physiologically acceptable carriers, excipients or diluents. Such carriers may be nontoxic to recipients at the dosages and concentrations employed. The preparation of such compositions may entail combining the modulator of an HDAC pathway with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin may be appropriate diluents. A modulator of an HDAC pathway may be formulated as a lyophilizate using appropriate excipient solutions (e.g., sucrose) as diluents. Appropriate dosages of a modulator of an HDAC pathway according to the present invention may be determined in trials. In accordance with appropriate industry standards, preservatives may also be added, such as benzyl alcohol.

A modulator of an HDAC pathway according to the present invention may be formulated as a tablet or lozenge in a conventional manner. For example, tablet or lozenge capsules may contain conventional excipients such as a binding compound, filler, lubricant, disintegrant or wetting compound. The binding compound may be syrup, accacia, gelatin, sorbitol, tragacanth, mucilage of starch or polyvinylpyrrolidone. The filler may be lactose, sugar, microcrystalline cellulose, maizestarch, calcium phosphate, or sorbitol. The lubricant may be magnesium stearate, stearic acid, talc, polyethylene glycol, or silica. The disintegrant may be potato starch or sodium starch glycollate. The wetting compound may be sodium lauryl sulfate. Tablets may be coated according to methods well known in the art.

A modulator of an HDAC pathway according to the present invention may also be a liquid formulation which may be an aqueous or oily suspension, solution, emulsion, syrup, or elixir. A modulator of an HDAC pathway according to the present invention may also be formulated as a dry product for constitution with water or other suitable vehicle before use. Such liquid preparations may contain an additive such as a suspending compound, emulsifying compound, nonaqueous vehicle or preservative. The suspending compound may be a sorbitol syrup, methyl cellulose, glucose/sugar syrup, gelatin, hydroxyethylcellulose, carboxymethyl cellulose, aluminum stearate gel, or a hydrogenated edible fat. The emulsifying compound may be lecithin, sorbitan monooleate, or acacia. The nonaqueous vehicle may be an edible oil, almond oil, fractionated coconut oil, oily ester, propylene glycol, or ethyl alcohol. The preservative may be methyl or propyl p-hydroxybenzoate, or sorbic acid.

A modulator of an HDAC pathway according to the present invention may also be formulated as a suppository, which may contain suppository bases which may be cocoa butter or glycerides. A modulator of an HDAC pathway according to the present invention may also be formulated for inhalation, which may be in a form such as a solution, suspension, or emulsion that may be administered as a dry powder or in the form of an aerosol using a propellant, such as dichlorodifluoromethane or trichlorofluoromethane. A modulator of an HDAC pathway according to the present invention may also be a transdermal formulation comprising an aqueous or nonaqueous vehicle which may be a cream, ointment, lotion, paste, medicated plaster, patch, or membrane.

A modulator of an HDAC pathway according to the present invention may also be formulated for parenteral administration, which may be by injection or continuous infusion. Formulations for injection may be in the form of a suspension, solution, or emulsion in oily or aqueous vehicles, and may contain a formulation compound such as a suspending, stabilizing, or dispersing compound. A modulator of an HDAC pathway according to the present invention may also be provided in a powder form for reconstitution with a suitable vehicle such as sterile, pyrogen-free water.

A modulator of an HDAC pathway according to the present invention may also be formulated as a depot preparation, which may be administered by implantation or by intramuscular injection. A modulator of an HDAC pathway according to the present invention may be formulated with a suitable polymeric or hydrophobic material (as an emulsion in an acceptable oil, for example), ion exchange resin, or as a sparingly soluble derivative (as a sparingly soluble salt, for example).

A modulator of an HDAC pathway according to the present invention may also be formulated as a liposome preparation. The liposome preparation may comprise a liposome which penetrates the cells of interest or the stratum corneum, and fuses with the cell membrane, resulting in delivery of the contents of the liposome into the cell. For example, the liposome may be as disclosed in U.S. Pat. No. 5,077,211 of Yarosh, U.S. Pat. No. 4,621,023 of Redziniak et al., or U.S. Pat. No. 4,508,703 of Redziniak et al. Other suitable formulations may employ niosomes. Niosomes are lipid vesicles similar to liposomes, with membranes consisting largely of non-ionic lipids, some forms of which are effective for transporting compounds across the stratum corneum.

### 3. Administration of a Modulator of an HDAC Pathway

A modulator of an HDAC pathway according to the present invention may be administered orally, parenterally, sublingually, transdermally, rectally, transmucosally, topically, via inhalation, via buccal administration, or combinations thereof. Parenteral administration may be intravenous, intraarterial, intraperitoneal, subcutaneous, intramuscular, intrathecal, or intraarticular. A modulator of an HDAC pathway according to the present invention may also be administered in the form of an implant, which allows slow release of the agent as well as a slow controlled i.v. infusion. For treating patients, such as mammals, including humans, according to the methods of the present invention, subcutaneous injection may be used because many modulators of an HDAC pathway are destroyed by the digestive process or otherwise ineffective if ingested.

### 4. Dosage of A Modulator of an HDAC Pathway

An amount sufficient to treat or ameliorate an effect of a polycystic or other related disease, such as a therapeutically effective amount of a modulator of an HDAC pathway according to the present invention, may vary with the nature of the condition being treated, the length of time that activity is desired, and the age and the condition of the patient, and ultimately may be determined by the attendant physician. The amount or dose of a modulator of an HDAC pathway according to the present invention that may be administered to a patient may also vary depending on a variety of factors known in the art (e.g., species, sex, age, weight, condition of the patient, desired response, nature of the condition, metabolism, severity of disease, side-effects). The desired dose may be conveniently administered in a single dose, or as multiple doses administered at appropriate intervals, for example as two, three, four or more subdoses per day. Multiple doses often are desired, or required.

A number of factors may lead to the modulator of an HDAC pathway being administered at a wide range of dosages. When given in combination with other therapeutics, the dosage of the modulator of an HDAC pathway of the present invention may be given at a relatively lower dosage. In addition, the use of a targeting substituent may allow the necessary dosage to be relatively low. A modulator of an HDAC pathway according to the present invention, however, may be administered at a relatively high dosage, which may be due to a factor such as low toxicity, high clearance, or low rates of processing. As a result, the dosage of a modulator of an HDAC pathway according to the present invention may be from about 1 ng/kg to about 1000 mg/kg. In general, however, doses employed for adult human treatment typically may be in the range of 0.0001 mg/kg/day to 0.0010 mg/kg/day, 0.0010 mg/kg/day to 0.010 mg/kg/day, 0.010 mg/kg/day to 0.10 mg/kg/day, 0.10 mg/kg/day to 1.0 mg/kg/day, 1.00 mg/kg/day to about 200 mg/kg/day. For example, the dosage may be about 1 mg/kg/day to about 100 mg/kg/day, such as, e.g., 2-10 mg/kg/day, 10-50 mg/kg/day, or 50-100 mg/kg/day. The dosage of the modulator of an HDAC pathway also may be about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

### 5. Murine Model for Polycystic Disease

The disclosed method of treating or ameliorating the effect of a polycystic disease with a modulator of an HDAC pathway according to the present invention may be based upon murine models. For example, those skilled in the art study human PKD using a PKD loss of function mutant mouse because various strains of mutant mice exhibit disease symptoms that parallel both ADPKD and ARPKD. See, e.g., “Polycystic Kidney Disease,” retrieved from the National Institute of Diabetes and Digestive and Kidney Diseases website. Mouse models for polycystic diseases may be possible, in part, because the molecular pathways underlying the pathology of PKD cyst formation are shared by both mice and humans. Polycystin-1 and polycystin-2, which are products of the PKD-1 and PKD-2 genes, respectively, may exhibit the same kidney cell localization in mice and humans (Cano et al., Development, 2004; 131:3457-67).

Studies using PKD mouse mutants aim at understanding the genetic and nongenetic mechanisms involved in cyst formation, and at discovering candidate compounds that inhibit cyst formation in PKD mutants. As disclosed above, the inventors have discovered that a modulator of an HDAC pathway, such as for example, an HDACi, may be used to inhibit cyst formation associated with PKD.

The invention now being generally described, it will be more readily understood by reference to the following examples, which are included merely for purposes of illustration of certain aspects and embodiments of the present invention, and are not intended to limit the invention.

### EXAMPLES

### Example 1

### Antibodies

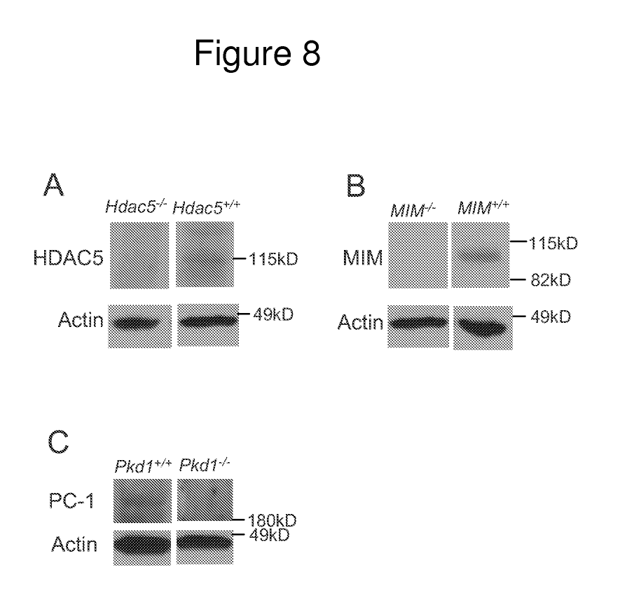

HDAC5 (Phospho-Ser498 human) antibody was obtained from Signalway Antibody (Pearland, Tex.). The non-phosphospecific anti-HDAC5 antibody and anti-MIM antibodies were purchased from Cell Signaling Technologies, Inc. (Denvers, Mass.). Anti-MEF2C antibody was from Santa Cruz Biotechnology, Inc. (Santa Cruz, Calif.). Anti-PC-1 antibody was a generous gift from Patricia Wilson (Wisconsin Medical College). Specific bands detected by the anti-HDAC5, anti-MIM, or anti-PC-1 antibody were verified against null adult kidneys or cell line (FIG. 8).

### Fluid Flow Experiments

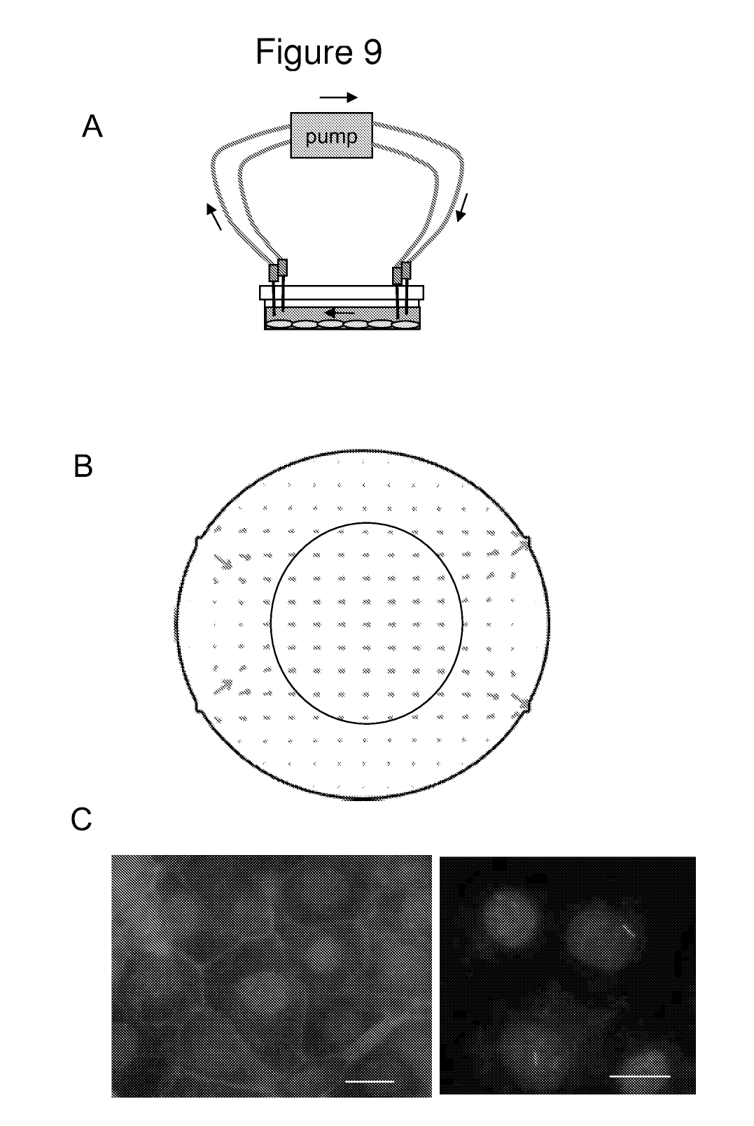

Confluent mouse embryonic kidney (MEK) cells were cultured for 1-2 additional days in the absence of interferon-γ to induce optimal differentiation (Nauli et al., 2003). Hanks' Balanced Salt Solution with 20.0 mM HEPES buffer (pH 7.4) and 1% bovine serum albumin was used for calcium imaging and cell culture media for the other experiments. Fluid flow was applied at 0.2 ml/min using a peristaltic pump in 35 mm dishes or 6-well plates, with tubings connected through two 18 Gauge needles at opposing sides of a well (FIG. 9A). Time-lapse imaging was performed on an Axiovert 200M inverted microscope (Karl Zeiss Microlmaging Inc., Thornwood, N.Y.) equipped with a 20×/0.8 NA Plan-Apochromat objective, a Pecon XL-3 environmental chamber (PeCon GmbH, Erbach, Germany) and an AxioCam HSm r1.1 digital monochrome camera (Karl Zeiss). In drug treatments, PMA (12-myristate 13-acetate) (Sigma-Aldrich, St. Louis, Mo.) was used at 100 nM, GÖ6983 (Sigma-Aldrich) at 10 μM, ionomycin (Sigma-Aldrich) at 1 μM, and GdCl₃ (Sigma-Aldrich) at 20 μM.

Computation of the flow field was performed with COMSOL Multiphysics finite element software (COMSOL Inc., Burlingame, Mass.), and the result is presented in FIG. 9B. The total inflow is characterized by the flow rate F=0.2 ml/min which is nearly evenly distributed over the rectangular cross-section with dimensions 2 cm×0.5 cm, i.e., over the area A=1 cm². The average velocity (v) is computed as v=F/A=0.2 cm/min=30 μm/s, with a calculated fluid shear stress to be 0.020 dyn/cm². The standard deviation was estimated by integration of the calculation presented above to be equal to 8 μm/s or 0.0053 dyn/cm₂.

### Microarray Analysis

Biotinylated cRNA was prepared from 5 μg total RNA using the standard Affymetrix one-cycle target labeling protocol (Affymetrix, Santa Clara, Calif.). Samples were assayed using Affymetrix GeneChip Mouse Genome 430 2.0 arrays consisting of probe sets representing over 39,000 transcripts based on the Unigene database (Build 107 from June 2002). Data was analyzed using the R statistical environment. Affymetrix CEL files were processed and normalized using RMA (Irizarry et al., 2003). The linear modeling package Limma (Azzam et al., 2004) was used to derive gene expression coefficients using genotype and presence or absence of fluid flow as factors, and to identify differentially expressed genes. The data was also examined and characterized with GCOS (Affymetrix) and Partek (St. Louis, Mo.) ANOVA.

### FLAG-MEF2C and HDAC5-GFP Constructs

FLAG-MEF2C (SEQ ID NO: 1) was made by inserting the Mef2C coding sequence with stop codon (TAA) (SEQ ID NO: 2) into p3XFLAG-Myc-CMV™-24 (Sigma-Aldrich) cut by KpnI and BamHI. HDAC5-GFP (SEQ ID NO: 3) was made by inserting the HDAC5 coding sequence (without stop codon) (SEQ ID NO: 4) into pAcGFP1-N1 (Clonetech, Mountain View, Calif.), cut by XhoI and HindIII.

### Chromatin IP and Luciferase Assay

Chromatin was prepared from FLAG-MEF2C or FLAG-HDAC5-expressing cells as described in Upstate protocol (MCPROT0407, available from Milipore's TECH LIBRARY, Milipore, Billerica, Mass.), with a cross-linking time of 15 min at 25° C. and sonication to an average length of 200-700 bp. ChIP was performed using anti-Flag M2 monoclonal antibody (Sigma). Samples were analyzed by PCR. Primers for MIM: forward, 5′-CCAGCCAGGGTTGCCATAGCCAC-3′ (SEQ ID NO: 5) and reverse, 5′-TGACTCTTGCAAACGGTTCAATTAGGTGC-3′ (SEQ ID NO:6), which flank a 1 kb region in the MIM promoter that harbor a potential MEF2 binding motif.

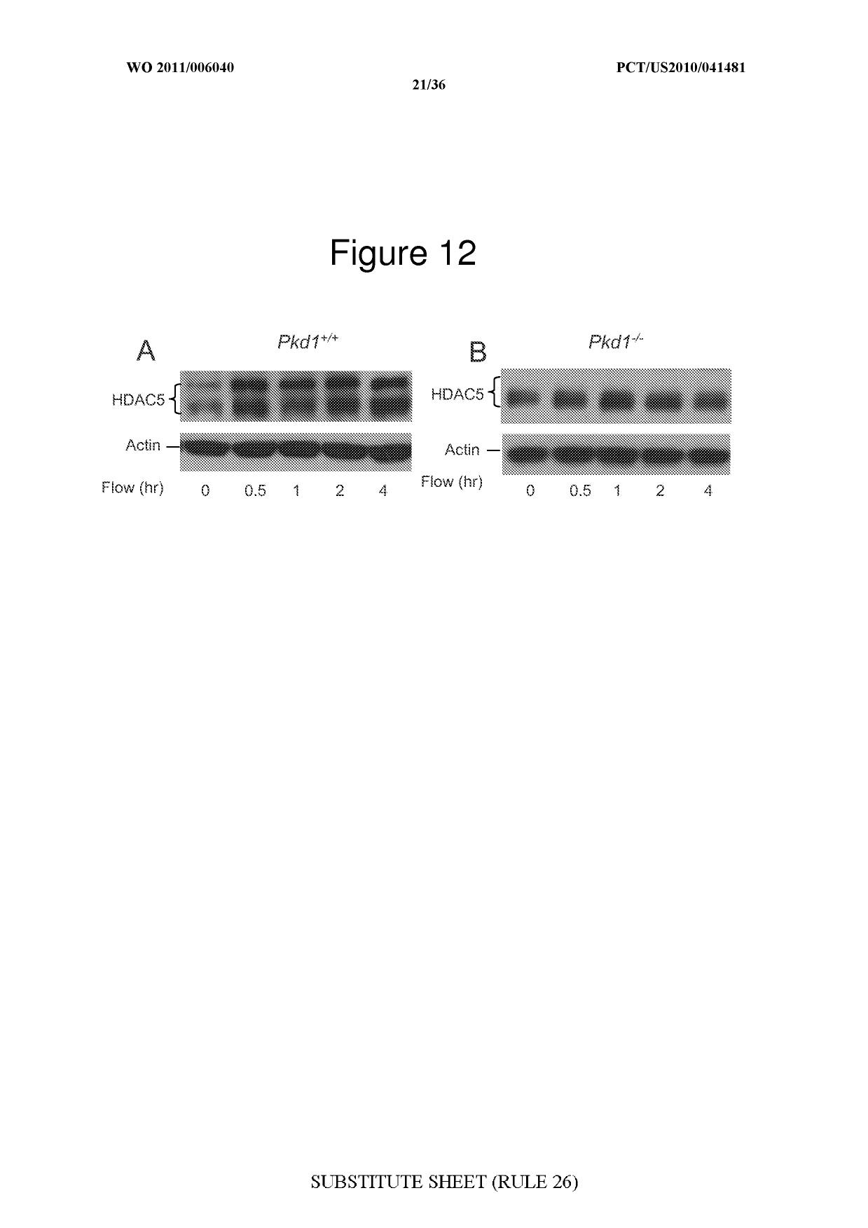

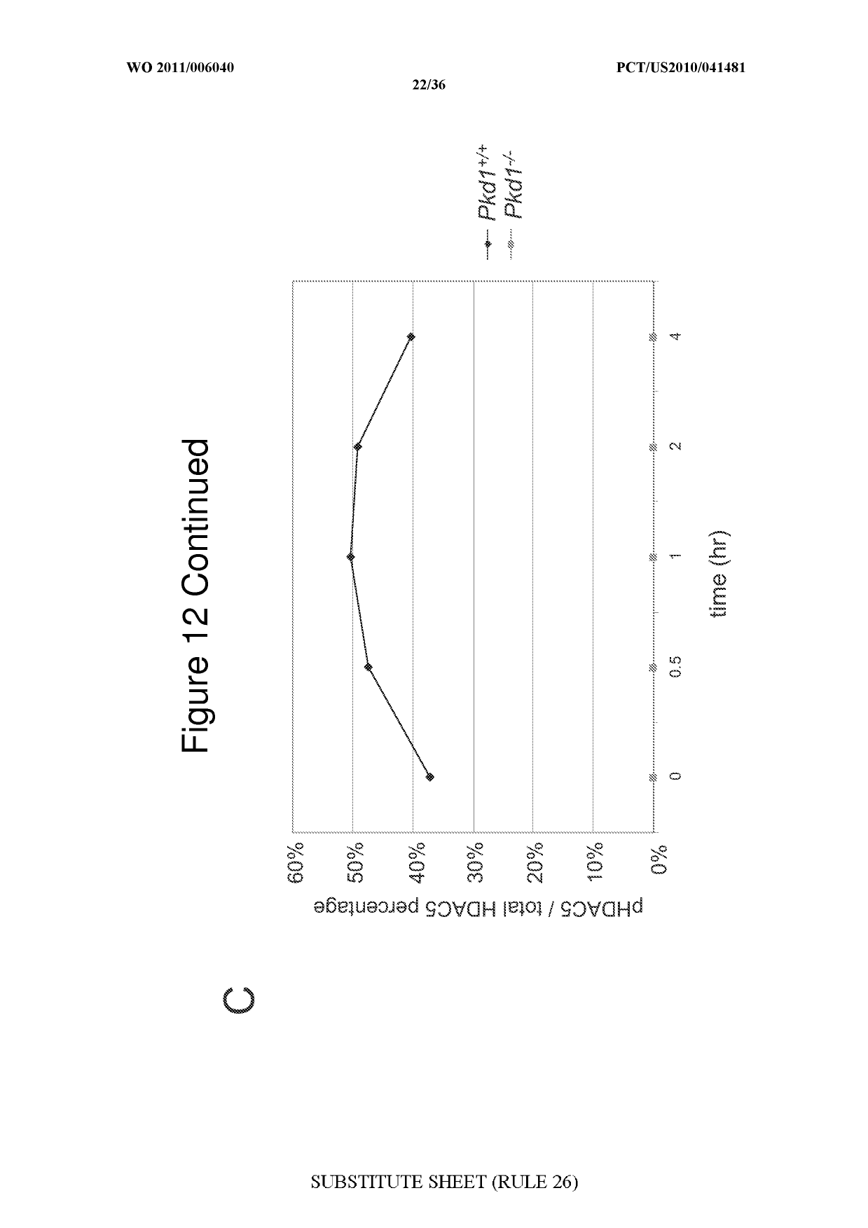

To determine if MEF2C is important for MIM expression, MEF2C siRNA was introduced into the wild-type MEK cells. Four different MEF2C RNAi sequences were pooled (SEQ ID NOs: 7-10). The MEF2C siRNA knocked down MEF2C expression, as determined by qPCR, to 38% of that in cells transfected with a control siRNA, and likewise MIM expression was reduced to 40% of that in the control cells (FIG. 12B). The primers and the probes used for qPCR are set forth below in Table 1.

TABLE 1

| | Sequence | SEQ ID NO |
|---|---|---|
| MEF2C forward | CCTGGCAGCAAGAACACGAT | 11 |
| MEF2C reverse | GACTGAGCCGACTGGGAGTTAT | 12 |
| MEF2C probe | CCATCAGTGAATCAAA | 13 |
| MIM forward | GCTCAGCTCTCACCAGAATGTG | 14 |
| MIM reverse | CGATCAAAGCACTGGAGAACTG | 15 |
| MIM probe | ATGCGGCACCGGAG | 16 |
| HDAC5 forward | GCTCATCCTGCGCAACAA | 17 |
| HDAC5 reverse | GAATTCCTGGAGCCTCAGCTT | 18 |
| HDAC5 probe | CAAAGAGAGTGCCATCG | 19 |
| GATA6 forward | GCTCATCCTGCGCAACAA | 20 |
| GATA6 reverse | GAATTCCTGGAGCCTCAGCTT | 21 |
| GATA6 probe | CAAAGAGAGTGCCATCG | 22 |

A luciferase assay was carried out 54 hours post-transfection using a commercial kit (Promega Corp., Madison, Wis.) and an Orin microplate luminometer (Berthod Technologies U.S.A. LLC, Oak Ridge, Tenn.). 200 ng of pRL-TK renilla luciferase was included in each transfection as internal control.

### RNA Interference

The oligonucleotide sequences used for Mef2c RNA interference (Dharmacon) are as follows: 5′-GAGGAUCACCGGAACGAAU-3′ (SEQ ID NO: 23); 5′-UAGUAUGUCUCCUGGUGUA-3′ (SEQ ID NO: 24); 5′-GAUAAUGGAUGAGCGUAAC-3′ (SEQ ID NO: 25); 5′-CCAGAUCUCCGCGUUCUUA-3′ (SEQ ID NO: 26). The oligonucleotides were transfected by using the DharmaFECT siRNA transfection reagent (Dharmacon Inc. Lafayette, Colo.).

MEK cells transduced with Pkd1 siRNA lentivitus (VIRHD/P/siPKD13297) and control lentivirus (Battini et al., 2008) were cultured for four weeks in complete medium in the presence of puromycin. Cells were harvested and PC1 expression was analyzed by immunoblot analysis.

### Mouse Strains and Treatment

All experiments involving higher vertebrates were approved by the Institutional Animal Care and Use Committee of the Stowers Institute for Medical Research.

All mouse strains used in this study were of the C57BL/6 background. ES cells (Baygenomics, Los Angeles, Calif.), carrying a trap in intron 1 of the MIM gene, were injected into the inner cell mass of C57BL6 blastocysts. Chimeric mice with a high (65%) contribution to coat color from 129 were bred for germline transmission. Mef2^{IoxP/IoxP} mice, originally provided by John Schwarz (Albany Medical Center, Albany, N.Y.), and SgIt2-Cre mice (CNRS-Orleans, France) were crossed to generate Mef2^{IoxP/+} SgIt2-Cre mice, which were then paired to generate Mef2^{IoxP/IoxP} SgIt2-Cre mice.

Pkd2^{+/-} mice were originally provided by S. Somlo (Yale University, New Haven, Conn.) and Hdac5^{-/-} mice by E. Olson (University of Texas Southwestern Medical Center, Dallas, Tex.). To obtain embryos of various genotypes, the mutant mice were paired and the pregnant females were sacrificed on 18.5 DPC to collect embryonic kidneys. For TSA treatment experiments pregnant Pkd2^{+/-} females paired with Pkd2^{+/-} males were subcutaneously injected daily, from 10.5 dpc to 18.5 dpc with 0.5 μg TSA (Biomol, Enzo Life Sciences Inc., Farmingdale, N.Y.) per gram mouse body weight or DMSO (solvent of TSA) in phosphate buffered saline (PBS). At the end of this treatment, females were sacrificed and embryonic kidneys were collected.

### Example 2

### Transcriptional Profiling to Identify Potential Targets of PC-1-Dependent Mechanosenation

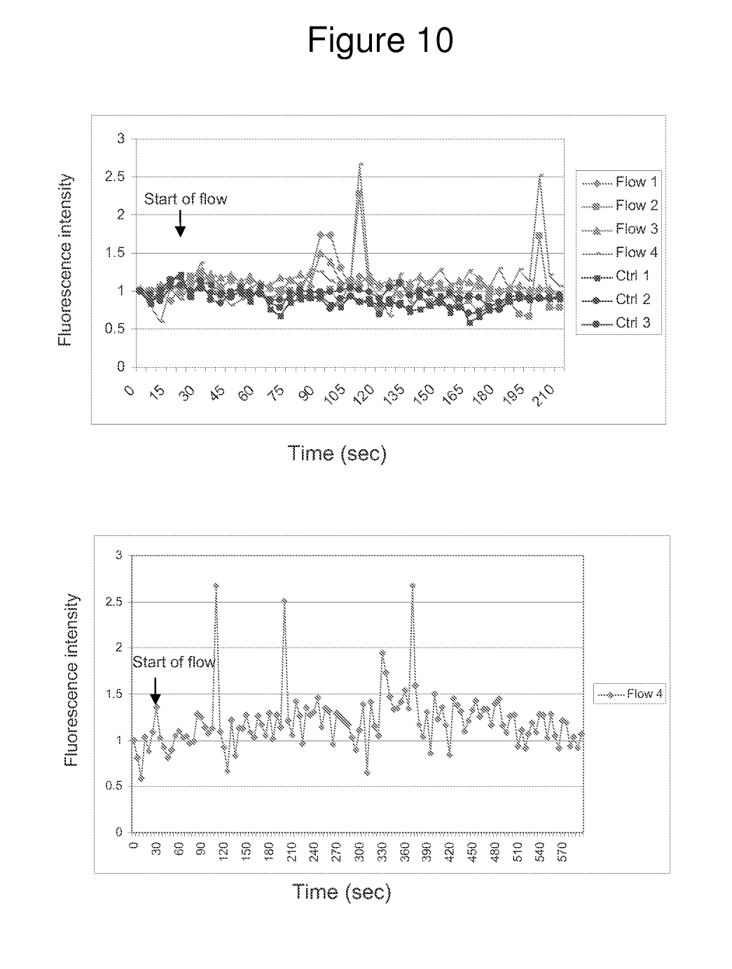

In order to identify pathways downstream of polycystins and the calcium signal in response to fluid shear stress, an in vitro system where fluid was applied using a peristaltic pump at a flow rate of 0.2 ml/min was established. The flow rate correlates to an average flow rate of 30 μm/sec, or a calculated shear stress of 0.020 dyn/cm², across polarized mono-layers of kidney epithelial cells grown on collagen coated coverslips in 35 mm culture dishes or 6-well plates (FIGS. 9A and 9B). This rate is within the range of flow rates observed in renal tubules and in line with previous work examining the effect of fluid flow on calcium channel activation in epithelial cells (Praetorius and Spring, 2001). To monitor calcium fluxes, GCAMP-2, a genetically coded calcium sensor that produces high signal to noise at physiological calcium concentrations, was used (Nakai et al., 2001; Tallini et al., 2006). In transfected cells expressing GCAMP-2 at moderate (but not extremely high) levels, fluid flow using the setup described above induced calcium transients within 60-120 seconds after the start of the flow, and in several cells imaged repeated calcium transients about 2 minutes apart were observed (FIG. 10). The duration of the calcium peaks was in the range of 10-20 seconds, similar to that from single-cell measurements in a recent study using a Fura-2 calcium sensor in Dolichos biflorus agglutinin (DBA)-positive MEK cells (Li et al., 2007).

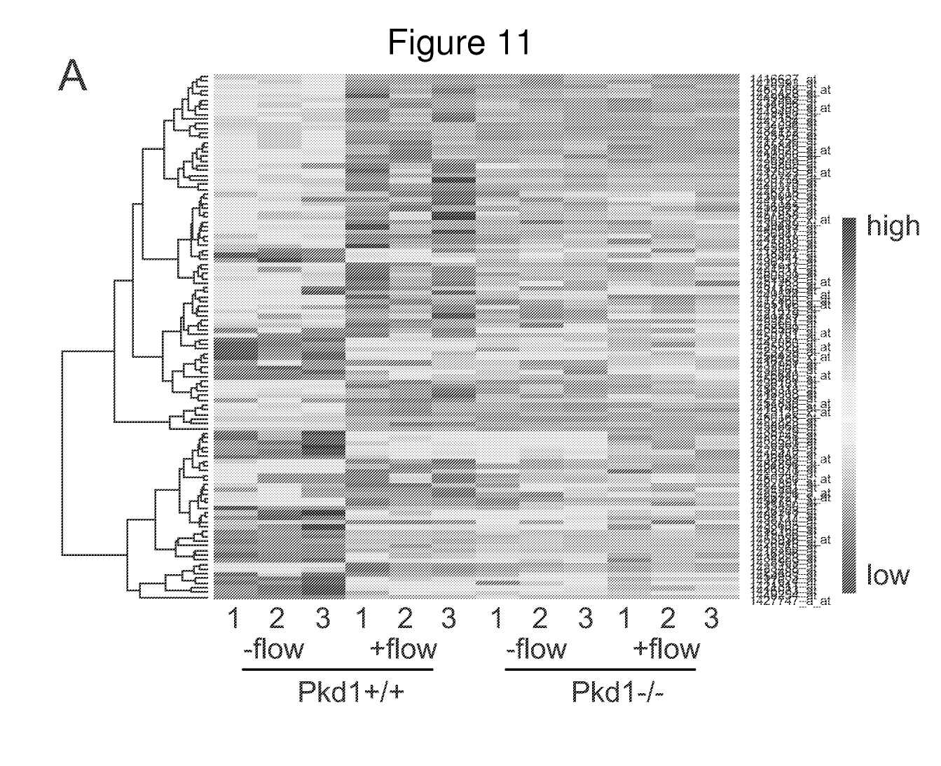

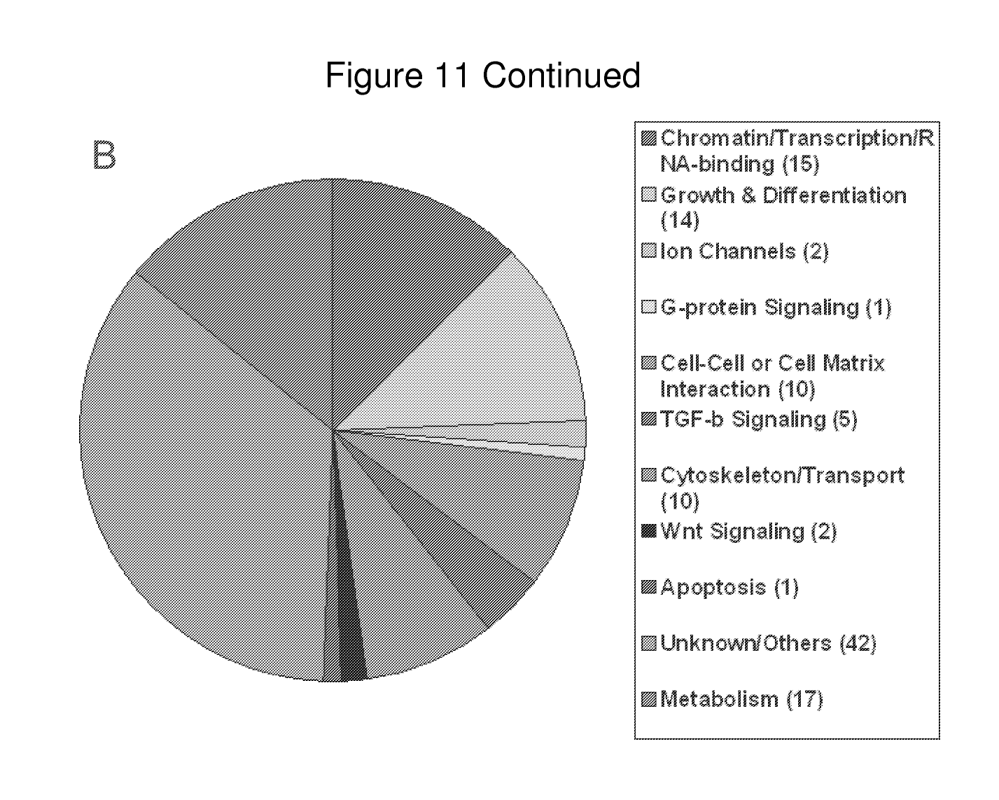

Using this experimental setup, an expression microarray analysis was performed to identify genes whose expression levels were altered in response to fluid flow in a PC-1 dependent manner. To this end, Pkd1^{+/+} and Pkd1^{-/-} mouse embryonic kidney (MEK) cell lines were used, which were established previously from the collecting ducts (DBA-positive) of SV40 large T antigen-immortalized Pkd1^{+/+} and Pkd1^{-/-} mouse embryos of the same genetic background (Li et al., 2005b; Nauli et al., 2003). These cells were grown confluent and differentiated in culture for 1-2 days, at which point cilia were clearly present in about 40% of the cells (FIG. 9C). Analysis of triplicate RNA samples from independent experiments using an Affymetrix mouse gene chip allowed for the identification of genes that were differentially regulated in response to fluid flow between Pkd1^{+/+} cells and Pkd1^{-/-} cells. Two different methods of analysis (ANOVA and Limma) were performed, which identified overlapping sets of genes encompassing a wide range of cellular functions, such as G-protein signaling, cytoskeleton regulation, cell cycle control, cell-matrix interactions, transcriptional regulation, etc. (FIG. 11 and Table 2 below).

TABLE 2
Genes identified by microarray analysis whose expression was up-regulated
in response to fluid flow in a Pkd1-dependent manner are listed in
the table below. Shown are relative expression levels in Pkd1^{+/+} MEK cells
or Pkd1^{-/-} MEK cells without or with flow (F) stimulation.

| | | | | | |
|---|---|---|---|---|---|
| | | | Pkd1^{+/+} | | Pkd1^{-/-} |
| Gene symbol | Gene title | Pkd1^{+/+} | F | Pkd1^{-/-} | F |

Metabolism

| | | | | | |
|---|---|---|---|---|---|
| CKb | creatine kinase, brain | 2.71 | 5.47 | 1.03 | 1 |
| Gda | guanine deaminase | 60.01 | 108.9 | 1 | 1.26 |
| Inpp5a | inositol polyphosphate- | 1.03 | 1.65 | 1.24 | 1 |
| | 5-phosphatase A | | | | |
| Nudt6 | nudix (nucleoside | 3.41 | 10.54 | 1.71 | 1 |
| | diphosphate linked | | | | |
| | moiety X)-type motif 6 | | | | |
| Obfc1 | oligonucleotide/oligosac | 1.43 | 2.27 | 1 | 1.06 |
| | charide-binding fold | | | | |
| | containing 1 | | | | |
| Tiparp | TCDD-inducible | 1.16 | 1.81 | 1 | 1.11 |
| | poly(ADP-ribose) polymerase | | | | |
| Upp1 | uridine phosphorylase 1 | 1 | 2.05 | 2.9 | 2.57 |
| Acsbg1 | acyl-CoA synthetase | 14.44 | 40.41 | 1 | 1.26 |
| | bubblegum family member 1 | | | | |
| Ptgs1 | prostaglandin- | 6.12 | 12.02 | 1.19 | 1 |
| | endoperoxide synthase 1 | | | | |
| Mrpl33 | mitochondrial ribosomal | 1.01 | 1.9 | 1 | 1.26 |
| | protein L33 | | | | |
| Txnip | thioredoxin interacting | 1 | 1.94 | 1.22 | 1.4 |
| | protein | | | | |
| HK2 | hexokinase 2 | 1 | 2.06 | 1.5 | 1.19 |
| Ptgs1 | prostaglandin- | 6.12 | 12.02 | 1.19 | 1 |
| | endoperoxide synthase 1 | | | | |
| Fabp4 | fatty acid binding | 4.02 | 22.5 | 1.1 | 1 |
| | protein 4, adipocyte | | | | |
| Mgat5 | mannoside | 1 | 1.56 | 1.1 | 1.27 |
| | acetylglucosaminyltransferase | | | | |
| | 5 | | | | |
| Acsl4 | acyl-CoA synthetase | 1 | 1.69 | 1.81 | 2.1 |
| | long-chain family member 4 | | | | |
| St3gal1 | ST3 beta-galactoside | 1.71 | 2.76 | 1 | 1.02 |
| | alpha-2,3-sialyltransferase 1 | | | | |

Cytoskeleton/Transport

| | | | | | |
|---|---|---|---|---|---|
| MIM | missing in metastasis protein | 58.11 | 95.56 | 1.66 | 1 |
| Baiap2l1 | BAl1-associated protein | 1.13 | 1.96 | 1.14 | 1 |
| | 2-like 1 | | | | |
| Nup54 | nucleoporin 54 | 1.11 | 2.29 | 1.19 | 1 |
| Sept9 | septin 9 | 1.69 | 2.8 | 1 | 1.06 |
| Fmnl2 | formin-like 2 | 1 | 1.84 | 2.05 | 2.63 |
| Grasp | GRP1-associated | 1.24 | 2.32 | 1 | 1.15 |
| | scaffold protein | | | | |
| Mical1 | microtubule associated | 1 | 1.87 | 1.78 | 2.53 |
| | monoxygenase, calponin and | | | | |
| | LIM domain containing 1 | | | | |
| Fgd6 | FYVE, RhoGEF and PH | 1.24 | 2.32 | 1 | 1.14 |
| | domain containing 6 | | | | |
| Psen2 | presenilin 2 | 1.22 | 2.02 | 1.16 | 1 |
| Arhgap6 | Rho GTPase activating | 1.8 | 5.71 | 1.23 | 1 |
| | protein 6 | | | | |

Potential Wnt Signaling

| | | | | | |
|---|---|---|---|---|---|
| LOC2269 | similar to ALY | 2.59 | 24.31 | 2.26 | 1 |
| Tcf23 | transcription factor 23 | 4.98 | 13.12 | 1.07 | 1 |

Apoptosis

| | | | | | |
|---|---|---|---|---|---|
| Bmf | Bcl2 modifying factor | 1.16 | 3.84 | 1 | 1.19 |

Chromatin/Transcription/RNA-binding

| | | | | | |
|---|---|---|---|---|---|
| Fli1 | Friend leukemia integration 1 | 113.08 | 174.44 | 1 | 1.36 |
| Nr4a1 | nuclear receptor subfamily 4, | 1 | 2.11 | 1.33 | 1.85 |
| | group A, member 1 | | | | |
| Prrx1 | paired related homeobox 1 | 23.58 | 59.94 | 1.12 | 1 |
| Nab1 | Ngfi-A binding protein 1 | 1 | 1.54 | 1.33 | 1.57 |
| Arid5b | Modulator recognition | 1 | 1.68 | 1.05 | 1.27 |
| | factor 2(Mrf2) | | | | |
| Ddef2 | development and | 4.11 | 7.14 | 1.1 | 1 |
| | differentiation enhancing | | | | |
| | factor 2 | | | | |
| MEF2C | myocyte enhancer factor 2C | 11.57 | 24.43 | 1.69 | 1 |
| GATA6 | GATA binding protein 6 | 8.74 | 13.15 | 1 | 1.02 |
| Nova1 | neuro-oncological | 17.42 | 36.56 | 1.89 | 1 |
| | ventral antigen 1 | | | | |
| HDAC5 | histone deacetylase 5 | 1.87 | 3.38 | 1 | 1.32 |
| Mllt10 | Myeloid/lymphoid or | 1.26 | 2.05 | 1 | 1.07 |
| | mixed lineage-leukemia | | | | |
| | translocation to 10 homolog | | | | |
| | (Drosophila) (Mllt10) | | | | |
| LOC5462 | similar to transcription | 1.65 | 7.09 | 1 | 1 |
| | elongation factor B | | | | |
| | polypeptide 3 binding | | | | |
| | protein 1 | | | | |
| H1f0 | H1 histone family, member 0 | 1 | 1.88 | 1.9 | 2.28 |
| H1fx | H1 histone family, member X | 1 | 1.54 | 1.58 | 1.54 |
| Smarcb1 | SWI/SNF related, matrix | 1.24 | 2.61 | 1 | 1.08 |
| | associated, actin dependent | | | | |
| | regulator of chromatin, | | | | |
| | subfamily b, member 1, | | | | |
| | mRNA | | | | |

Growth & Differentiation

| | | | | | |
|---|---|---|---|---|---|
| Rasa3 | RAS p21 protein activator 3 | 1.38 | 2.18 | 1 | 1.01 |
| Slfn2 | schlafen 2 | 2.81 | 6.65 | 1 | 1.27 |
| Cul3 | Cullin 3 | 1 | 1.82 | 1.75 | 2.06 |
| Trib2 | tribbles homolog 2 | 1 | 2.9 | 2.15 | 2.03 |
| | (Drosophila) | | | | |
| Cxcl1 | chemokine (C-X-C motif) | 1 | 1.83 | 2.97 | 4.24 |
| | ligand 1 | | | | |
| Dusp6 | dual specificity phosphatase 6 | 2.05 | 3.7 | 1.08 | 1 |
| Fbxo2 | F-box only protein 2 | 2 | 3.05 | 1 | 1.18 |
| Igfbp4 | insulin-like growth factor | 82.19 | 163.87 | 2.17 | 1 |
| | binding protein 4 | | | | |
| Prkg2 | Protein kinase, cGMP- | 3.41 | 8.35 | 1 | 1.5 |
| | dependent, type II (Prkg2) | | | | |
| Adcy7 | adenylate cyclase 7 | 5.94 | 12.38 | 1 | 1.37 |
| Ndrg2 | N-myc downstream | 8.9 | 28.86 | 1.62 | 1 |
| | regulated gene 2 | | | | |
| Mmd | monocyte to macrophage | 1.91 | 4.69 | 1 | 1.2 |
| | differentiation-associated | | | | |
| Amhr2 | anti-Mullerian hormone | 1 | 3.26 | 1.35 | 1.31 |
| | type 2 receptor | | | | |
| Spry2 | sprouty homolog 2 | 1.96 | 3.43 | 1 | 1.07 |
| | (Drosophila) | | | | |

Ion Channels

| | | | | | |
|---|---|---|---|---|---|
| Slc4a4 | solute carrier family 4 (anion | 4.36 | 8.43 | 1.35 | 1 |
| | exchanger), member 4 | | | | |
| Kcnh2 | potassium voltage-gated | 1 | 2.19 | 1.13 | 1.32 |
| | channel, subfamily H, | | | | |
| | member 2 | | | | |

TGF-beta signaling

| | | | | | |
|---|---|---|---|---|---|
| Ltbp1 | latent transforming growth | 2.28 | 4.29 | 1.09 | 1 |
| | factor beta binding protein 1 | | | | |
| Nrn1 | neuritin 1 | 9.09 | 17.11 | 1.82 | 1 |
| Bambi | BMP and activin membrane- | 7.39 | 15.02 | 1.74 | 1 |
| | bound inhibitor, homolog | | | | |
| | (Xenopus laevis) | | | | |
| Htra3 | HtrA serine peptidase 3 | 8.16 | 13.46 | 1 | 1.22 |
| Chst11 | carbohydrate | 4.88 | 8.21 | 1.09 | 1 |
| | sulfotransferase 11 | | | | |

G-protein Signaling

| | | | | | |
|---|---|---|---|---|---|
| Rgs3 | Regulator of G-protein | 1.14 | 2.72 | 1.53 | 1 |
| | signaling 3 | | | | |

Cell-Cell or Cell-matrix Interaction

| | | | | | |
|---|---|---|---|---|---|
| Clec1a | C-type lectin domain | 8.28 | 27.41 | 2.92 | 1 |
| | family 1, member a | | | | |
| Itga7 | integrin alpha 7 | 15.68 | 37.99 | 1.65 | 1 |
| Mgp | matrix Gla protein | 1 | 9.91 | 1.56 | 1.27 |
| Vcam1 | vascular cell adhesion | 15.26 | 28.99 | 1.32 | 1 |
| | molecule 1 | | | | |
| Mmp13 | matrix metallopeptidase 13 | 12.48 | 34.27 | 1 | 1.47 |
| Emp2 | epithelial membrane protein 2 | 1 | 2.21 | 1.17 | 1.25 |
| Itga10 | integrin, alpha 10/similar to | 2.42 | 5.47 | 1.09 | 1 |
| | integrin, alpha 10 precursor | | | | |
| Tm4sf1 | transmembrane 4 superfamily | 10.31 | 17.21 | 1 | 1.46 |
| | member 1 | | | | |
| Itgb2 | integrin beta 2 | 13.28 | 29.11 | 1.39 | 1 |
| Tspan32 | tetraspanin 32 | 6.33 | 12.35 | 1 | 1.43 |

Unknown/Others

| | | | | | |
|---|---|---|---|---|---|
| Cmklr1 | RIKEN cDNA | 12.4 | 37.11 | 1 | 1.44 |
| | 8430438D04 gene | | | | |
| 8430438D | RIKEN cDNA | 5.78 | 17.9 | 1 | 1.32 |
| | 8430438D04 gene | | | | |
| D630035 | RIKEN cDNA | 3.79 | 7.3 | 1 | 1.33 |
| | D630035O19 gene | | | | |
| Spint1 | serine protease inhibitor, | 8.59 | 19.79 | 1.43 | 1 |
| | Kunitz type 1 | | | | |
| Abca13 | ATP-binding cassette, sub- | 17.85 | 137.03 | 1.52 | 1 |
| | family A (ABC1), member 13 | | | | |
| D14Ertd6 | troponin T2, cardiac | 7.31 | 21.19 | 1 | 1.04 |
| 2610027C | RIKEN cDNA | 1 | 2.12 | 1.28 | 1.4 |
| | 2610027C15 gene | | | | |
| Antxr2 | anthrax toxin receptor 2 | 1 | 1.77 | 1.51 | 1.41 |
| AI447904 | expressed sequence | 1.64 | 7.03 | 2.09 | 1 |
| | AI447904 | | | | |
| 1810023F | RIKEN cDNA | 7.53 | 16.74 | 1.12 | 1 |
| | 1810023F06 gene | | | | |
| C030045D | RIKEN cDNA | 35.4 | 61.44 | 1.16 | 1 |
| | C030045D06 gene | | | | |
| Ifi203 | interferon activated gene | 16.61 | 39.09 | 1 | 1.22 |
| | 203/similar to interferon- | | | | |
| | inducible protein 203 | | | | |
| LOC5455 | similar to RIKEN cDNA | 1.82 | 3.62 | 1.03 | 1 |
| | B230218L05 gene | | | | |
| C3ar1 | complement component | 5.28 | 9.75 | 1.09 | 1 |
| | 3a receptor 1 | | | | |
| Ly6e | lymphocyte antigen 6 | 1 | 2.35 | 1.72 | 2.23 |
| | complex, locus E | | | | |
| BC037704 | cDNA sequence | 2.96 | 6.74 | 1 | 1.23 |
| | BC037704 | | | | |
| 18100110 | RIKEN cDNA | 1.21 | 5.11 | 1.1 | 1 |
| | 1810011O10 gene | | | | |
| BCO | tribbles homolog 2 | 47.47 | 79.02 | 1.6 | 1 |
| Loc25600 | (Drosophila) | | | | |
| 1110019c | RIKEN cDNA | 6.42 | 33.35 | 1 | 1.38 |
| | 1110019C06 gene | | | | |
| D8Ertd82 | DNA segment, Chr 8, | 3.24 | 5.44 | 1 | 1.12 |
| | ERATO Doi 82, expressed | | | | |
| Au020206 | expressed sequence | 2.02 | 3.06 | 1.06 | 1 |
| | AU020206 | | | | |
| Raet1a | retinoic acid early | 2.59 | 3.9 | 1.02 | 1 |
| | transcript 1, alpha | | | | |
| Ifnz | interferon zeta | 3.25 | 6.45 | 1 | 1.42 |
| Ifi27 | interferon, alpha- | 120.77 | 258.26 | 1.07 | 1 |
| | inducible protein 27 | | | | |
| BC022765 | cDNA sequence | 2.65 | 4.17 | 1.05 | 1 |
| | BC022765 | | | | |
| Isg20 | interferon-stimulated protein | 2.3 | 3.67 | 1 | 1.17 |
| C630004H | RIKEN cDNA | 3.71 | 5.73 | 1 | 1.17 |
| | C630004H02 gene | | | | |
| Glcci1 | Glucocorticoid induced | 1.33 | 2.57 | 1 | 1.13 |
| | transcript 1 (Glcci1), | | | | |
| | transcript variant 1, mRNA | | | | |
| Scyl1bp1 | SCY1-like 1 binding | 1.02 | 1.57 | 1 | 1.27 |
| | protein 1 | | | | |
| SepW1 | selenoprotein W, muscle 1 | 1.02 | 1.56 | 1 | 1.3 |
| 4930539P | RIKEN cDNA | 1 | 1.64 | 1.65 | 1.26 |
| | 4930539P14 gene | | | | |
| MGI:1930 | brain protein 17 | 3.81 | 5.97 | 1 | 1.08 |
| GM253 | CD300 antigen like family | 1.31 | 5.02 | 1.65 | 1 |
| | member B (Cd300lb), mRNA | | | | |
| Tcra | T-cell receptor alpha | 3.2 | 5.04 | 1.4 | 1 |
| | chain/RIKEN cDNA | | | | |
| | A430107P09 gene | | | | |
| Spp1 | secreted phosphoprotein 1 | 1 | 4.94 | 3.47 | 4.35 |
| Hmgb2 | High mobility group box 2, | 1 | 1.64 | 1.93 | 2.54 |
| | mRNA (cDNA clone | | | | |
| | MGC:6061 | | | | |
| | IMAGE:3489780) | | | | |
| 290034 | RIKEN cDNA | 1 | 1.53 | 1.12 | 1.47 |
| | 2900034E22 gene | | | | |
| LOC433777 | similar to Hypothetical | 20.83 | 127.4 | 1.68 | 1 |
| | protein DJ1198H6.2/similar | | | | |
| | to Hypothetical protein | | | | |
| | DJ1198H6.2/similar to | | | | |
| | Hypothetical | | | | |
| 2610203C20 | RIKEN cDNA | 1.18 | 2.41 | 1 | 1.33 |
| | 2610203C20 gene | | | | |
| AI467606 | expressed sequence | 1.85 | 5.41 | 1 | 1.21 |
| | AI467606 | | | | |
| C030034I22 | RIKEN cDNA | 2.4 | 4.14 | 1 | 1.44 |
| | C030034I22 gene | | | | |
| H2-T23 | histocompatibility 2, T | 3.76 | 6.4 | 1 | 1.45 |
| | region locus 23 | | | | |

### Example 3

### Fluid Flow Induces Hdac5 Phosphorylation, Nuclear Export and MEF2C Target Genes

The presence of histone deacetylase-5 (HDAC5) and myocyte enhancer factor-2C (MEF2C) among the PC-1-dependent, flow-induced genes was particularly intriguing (see Table 2 above), because these proteins were known to function in the same pathway in the control of cardiac hypertrophy in response to stress and calcium channel activation (McKinsey et al., 2002; Olson et al., 2006). MEF2 targets include not only structural proteins important for cardiac muscle differentiation, but also members of the MEF2 and class II HDAC families through positive feedback loops (Haberland et al., 2007; Wang et al., 2001). Based on the knowledge set forth above, it is reasoned that the presence of both MEF2C and HDAC5 among the fluid flow-induced genes could be a reflection of activation of MEF2-based transcription downstream of the calcium rise elicited by polycystins.

A well-known post-translational mechanism for stress-induced MEF2C activation in myocardial cells involves phosphorylation and nuclear export of HDAC5, which represses MEF2-dependent transcripts in the resting state (McKinsey et al., 2000). Kinases activated by stress-induced Ca²⁺ increase, such as CaM kinase and protein kinase C or D, phosphorylate HDAC5 at two 14-3-3 binding sites, an event that leads to disruption of HDAC5-MEF2C interaction and translocation of HDAC5 from the nucleus to the cytosol (McKinsey et al., 2002). HDAC5 dissociation from MEF2C frees a binding site for the histone acetyl transferase p300/CBP, an activator of MEF2C. Mutually exclusive interaction of MEF2C with HDAC5 or p300/CBP generates a binary switch for activation of MEF2C target genes (McKinsey et al., 2001).

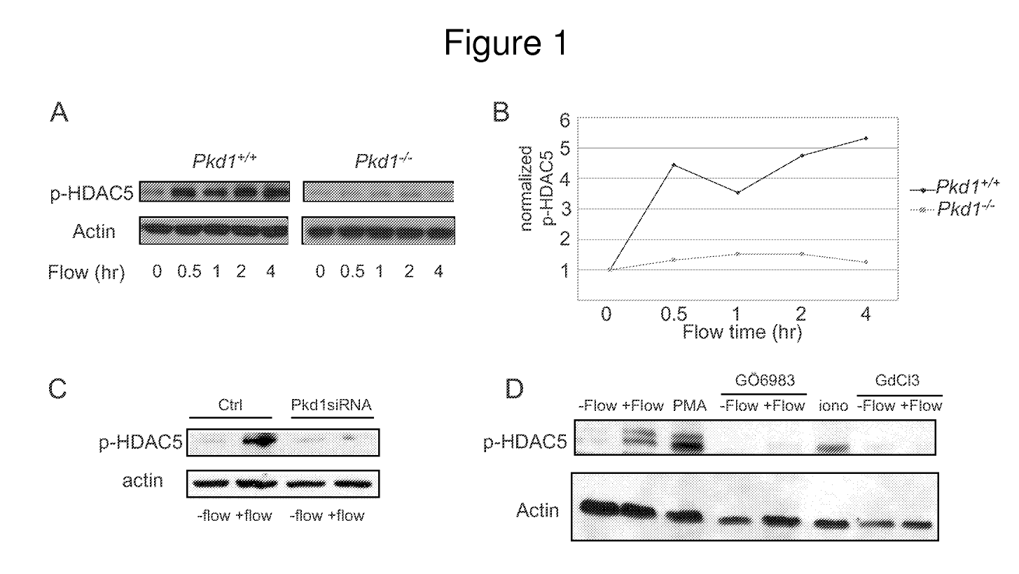

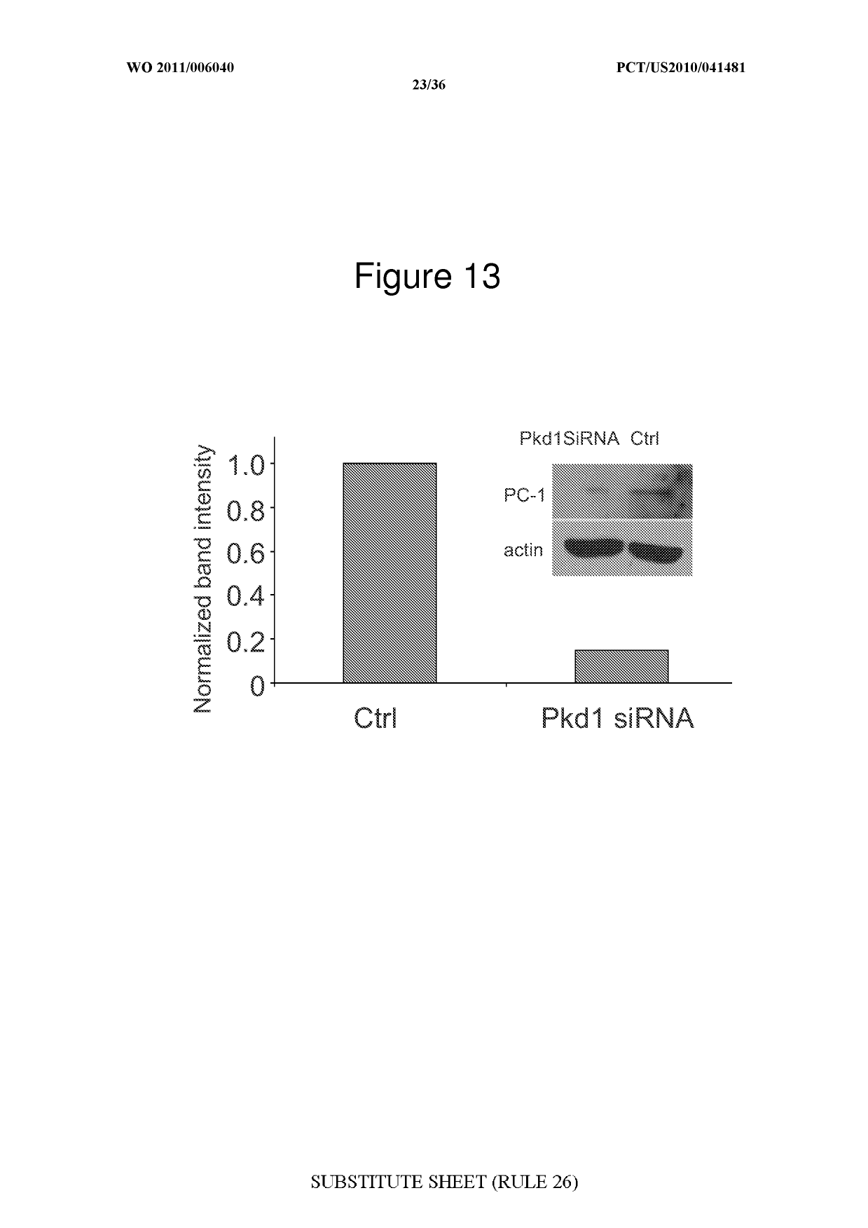

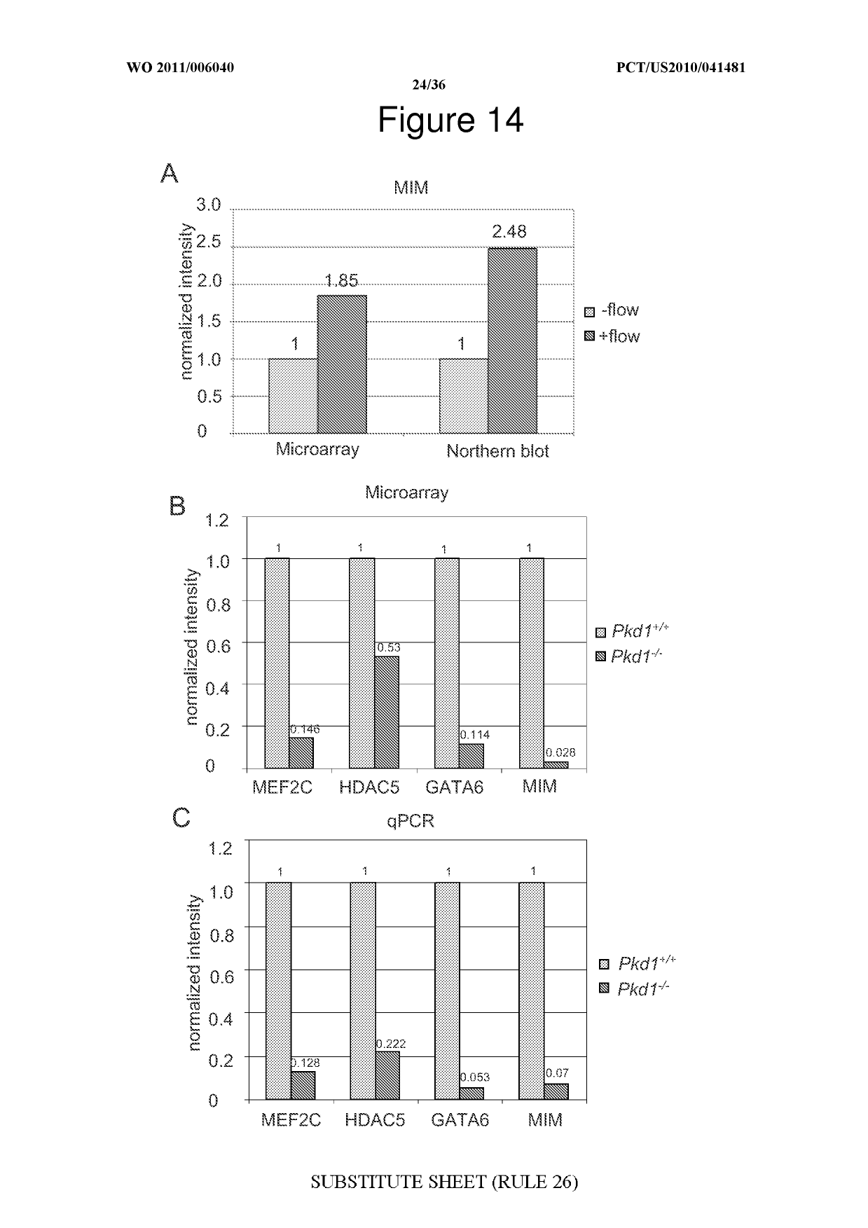

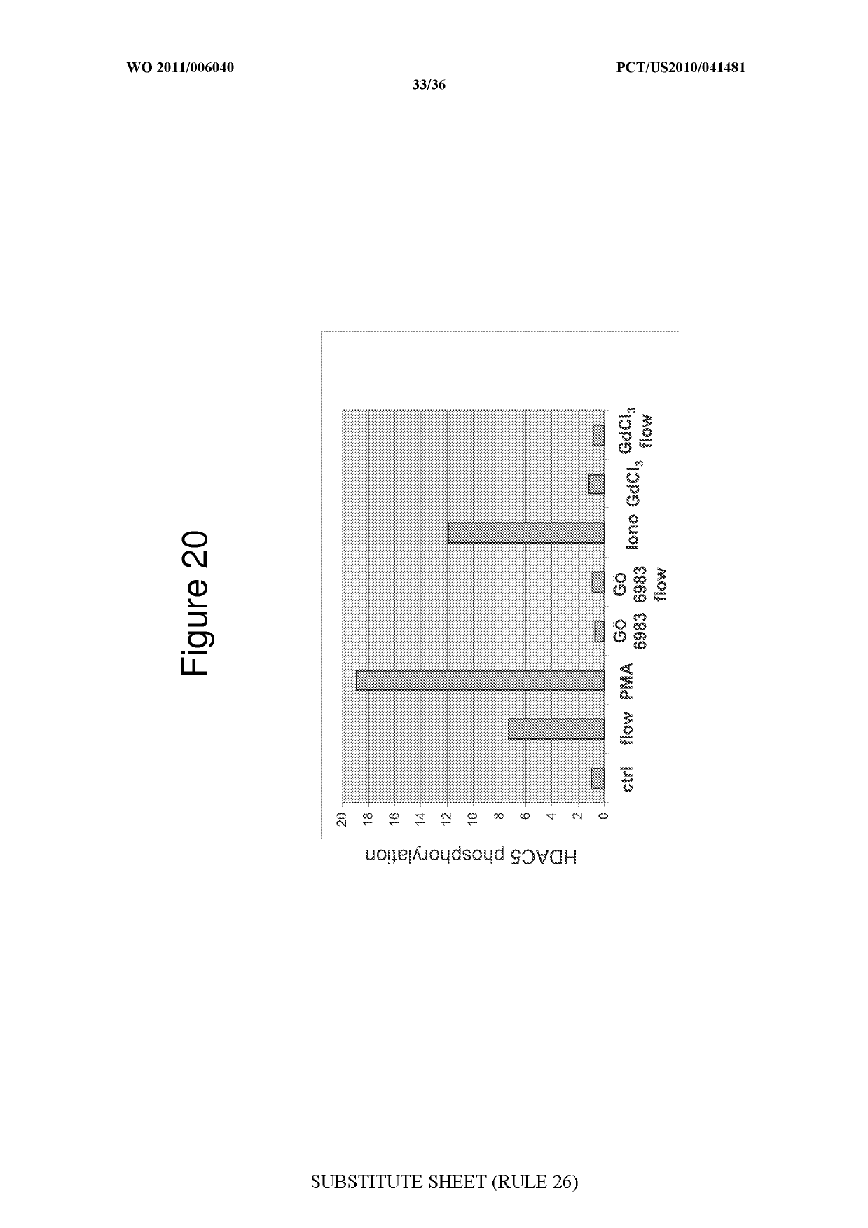

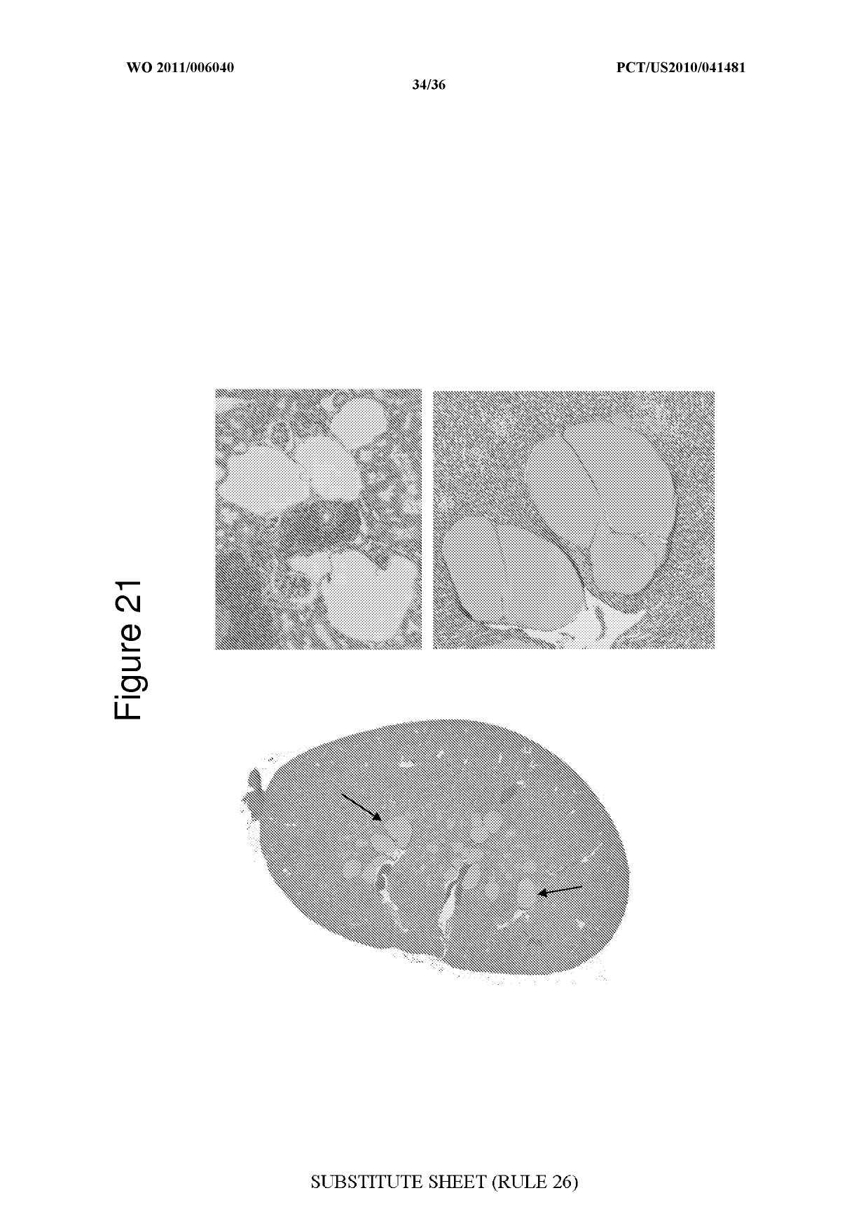

Whether fluid flow across monolayers of MEK cells led to endogenous HDAC5 phosphorylation was determined using a phospho-specific antibody against HDAC5 Ser489 site (equivalent of Ser498 of human HDAC5) (Bossuyt et al., 2008). While HDAC5 phosphorylation level was low prior to fluid flow onset, an increase in phosphorylation was observed 30 minutes after the start of fluid flow and maintained for several hours thereafter (FIGS. 1A and 1B). The increase in HDAC5 phosphorylation was accompanied by an increase in the level of total HDAC5 (FIGS. 12A, C), consistent with the flow-induced gene expression of HDAC5 observed in the microarray analysis (Table 2). Fluid flow-induced HDAC5 phosphorylation and increase in protein level were absent in Pkd1^{-/-} MEK cells (FIGS. 1A, 1B, 12B, and 12C) and blocked in wild-type MEK cells by Pkd1 siRNA (Battini et al., 2008) (FIG. 1C), which reduced PC-1 expression by 85% (FIG. 13), suggesting a direct role for PC-1 in the observed response. HDAC5 phosphorylation was also blocked by gadolinium (GdCl₃), an inhibitor of stretch-activated cation channels previously shown to inhibit fluid flow-induced calcium response (Praetorius et al., 2003). By contrast, HDAC5 phosphorylation was induced by the calcium ionophore, ionomycin, in the absence of fluid flow (FIG. 1D, see also FIG. 20 for the quantification of HDAC5 phosphorylation levels under various conditions). Treatment with PMA, an activator of protein kinase C(PKC), stimulated HDAC5 phosphorylation in the absence of fluid flow, while the PKC inhibitor GÖ6983 inhibited flow-induced HDAC5 phosphorylation (FIGS. 1D and 20). These data suggest that PC-1, calcium channel activity and a PKC isoform are required for fluid-flow induced HDAC5 phosphorylation.

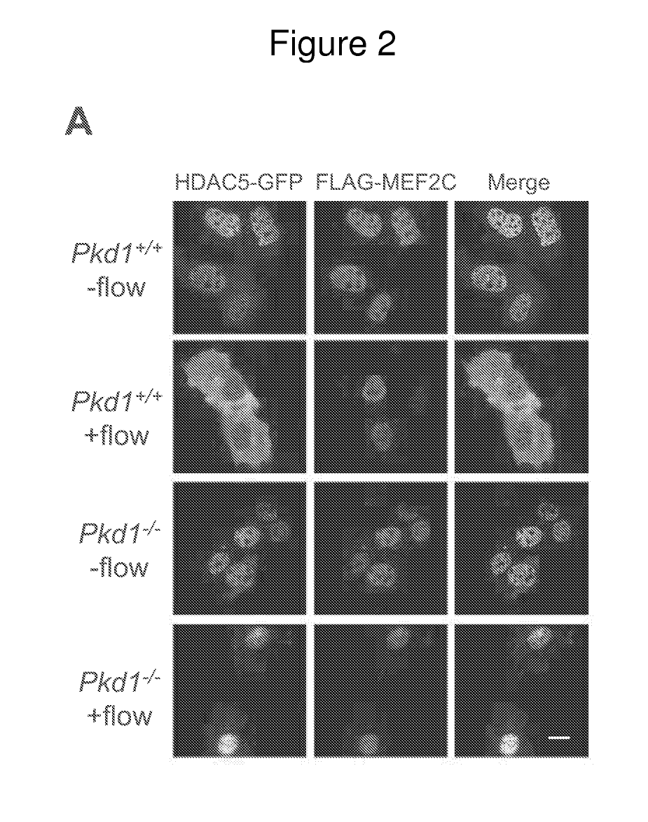

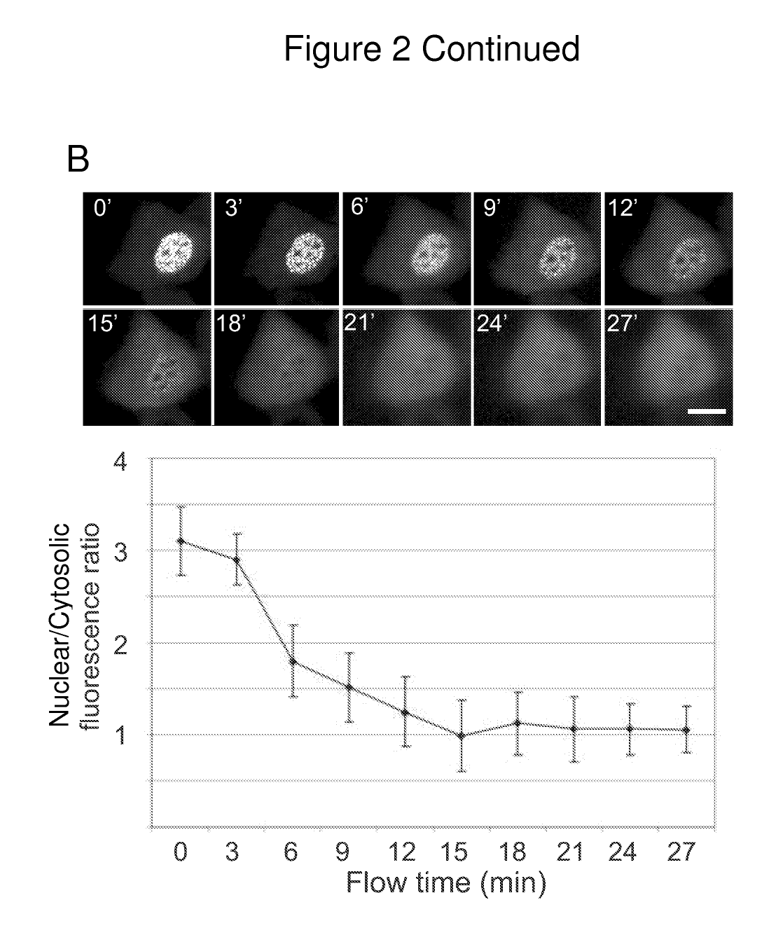

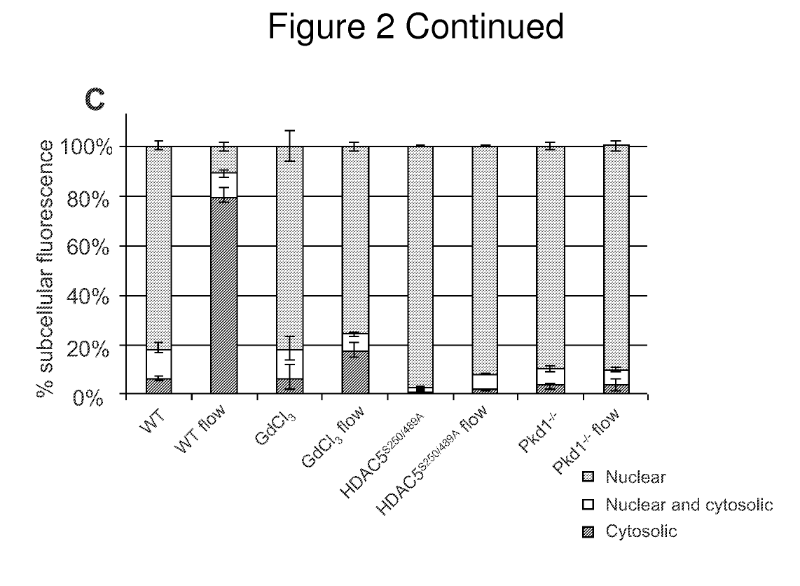

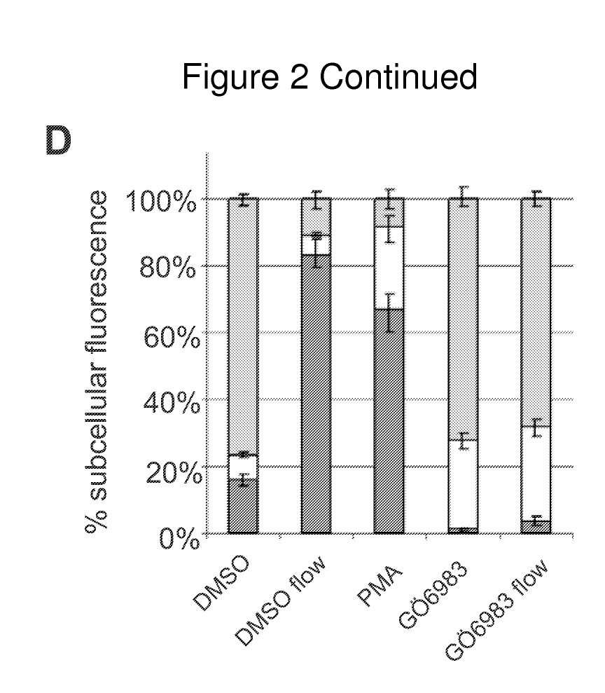

To determine whether HDAC5 was exported from the nucleus in response to fluid flow, FLAG-MEF2C and HDAC5-GFP constructs were co-transfected into Pkd1^{+/+} and Pkd1^{-/-} MEK cells, and both tagged proteins correctly localized to the nucleus. Stimulation of MEK cells with fluid flow for 30 min led to translocation of HDAC5-GFP, but not FLAG-MEF2C, from the nucleus in Pkd1^{+/+}. MEK cells, but not in Pkd1^{-/-} MEK cells (FIG. 2A). Time-lapse movies showed that HDAC5-GFP nuclear export initiated several minutes after the start of the flow stimulation and stayed in the cytoplasm for the duration of the observation (FIG. 2B). By contrast, a HDAC5-GFP mutant lacking the 14-3-3 phosphorylation sites (HDAC5^{S250/489A}) was not exported (FIG. 2C), consistent with a requirement for phosphorylation of these regulatory sites in fluid flow-induced nuclear export of HDAC5. HDAC5 nuclear export also failed in Pkd1^{-/-} MEK cells, cells treated with GdCl₃ (FIG. 2C), or the PKC inhibitor GÖ6983, while treatment with PMA stimulated HDAC5 nuclear exit even without fluid flow (FIG. 2D). These results suggest that the fluid flow-induced HDAC5 phosphorylation and nuclear export occurs in a PC-1 and calcium-dependent manner and PKC is an upstream kinase required for these events.

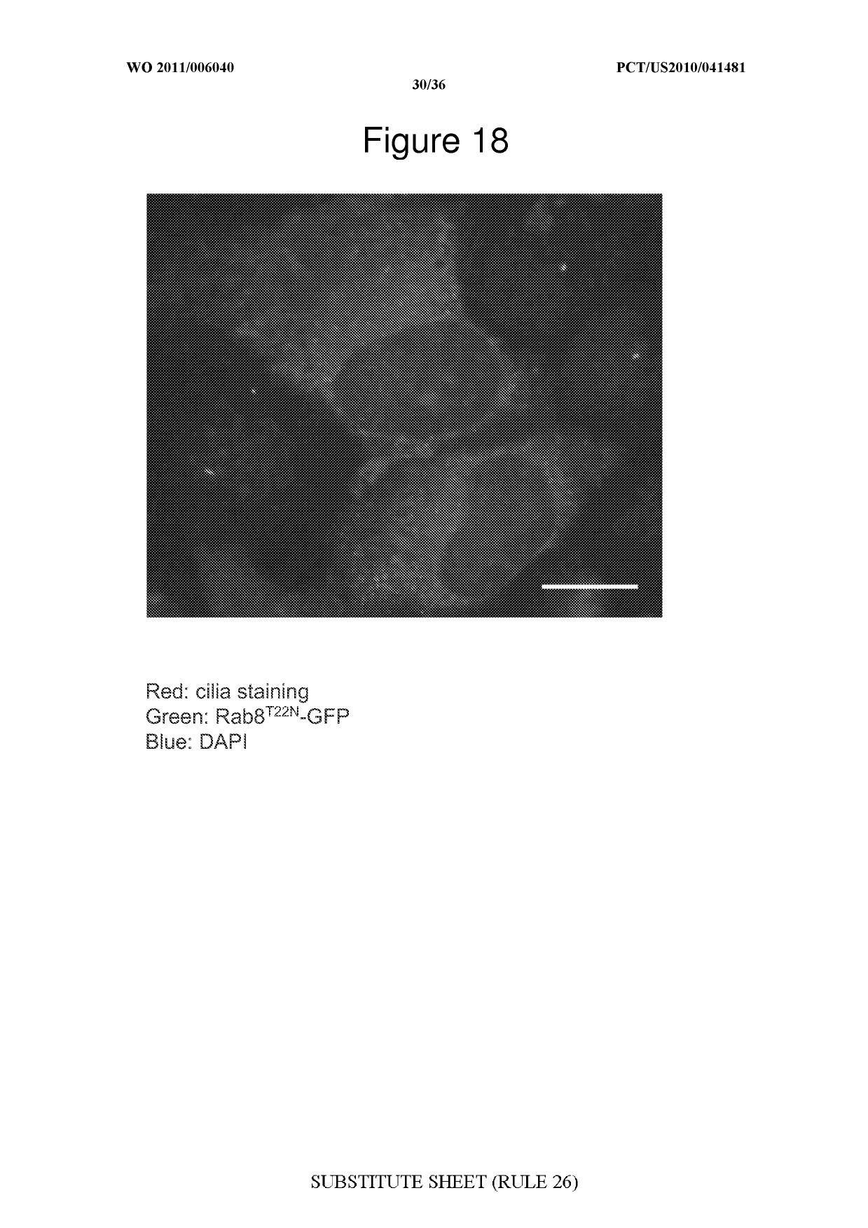

Because the mechanosensory function of polycystins is thought to be associated with their localization to the cilia, which may serve as an “antenna” probing fluid movement cross the apical surface, we tested if cilia were required for the flow-induced HDAC5 nuclear export. Using a dominant negative construct of Rab8 (Rab8^{T22N}), a Rab GTPase required for cilia extension (Nachury et al., 2007), cilia formation was blocked in 75% of the transfected MEK cells (FIG. 18). Surprisingly, HDAC5 nuclear export was unaffected in cells transfected with Rab8^{T22N} following fluid flow stimulation (FIG. 2C), suggesting that the observed phenomenon, while being PC-1-dependent, does not require cilia.

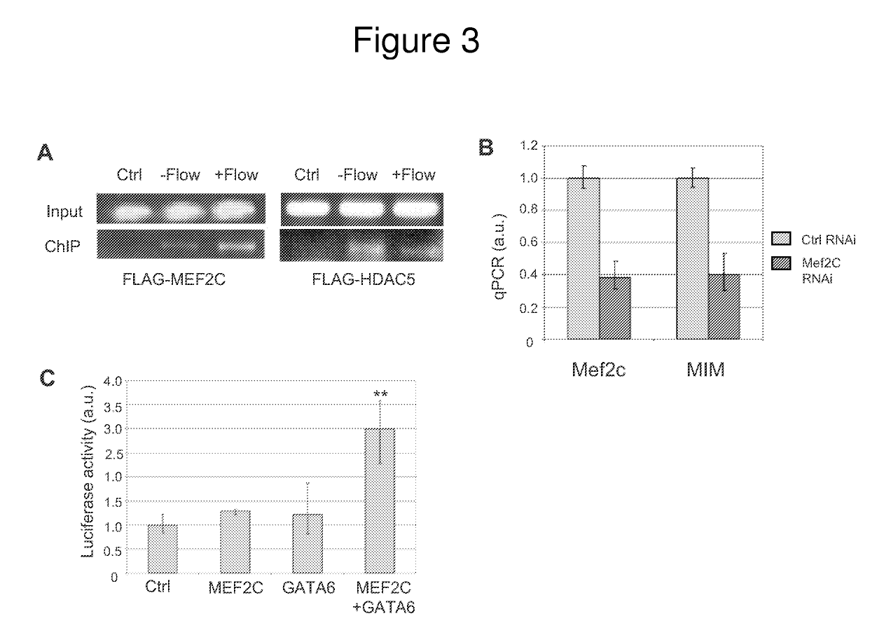

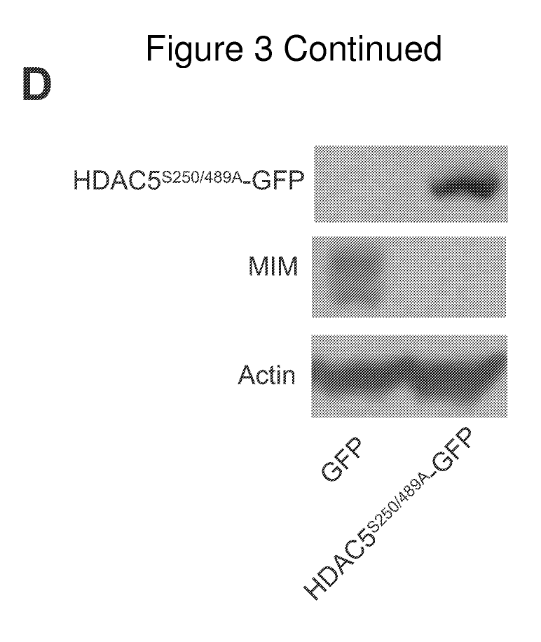

Because HDAC5 phosphorylation and nuclear export are key steps in the activation of MEF2C-based transcription, a potential target of MEF2C, a gene called Missing in Metastasis (MIM), was investigated. MIM was one of the fluid flow-induced genes in the microarray analysis, which was confirmed by Northern blot analysis (FIG. 13A). The probe used for the Northern blot was ATGCGGCACCGGAG (SEQ ID NO: 16). MIM is a multifunctional regulator of actin cytoskeletal dynamics (Machesky and Johnston, 2007) and was previously implicated in Sonic hedgehog (Shh) signaling (Callahan et al., 2004). Coincidentally, MIM was also among a list of potential targets of MEF2 in the heart, although its function in cardiac myocytes has not been characterized (van Oort et al., 2006). To determine if MEF2C indeed associates with the promoter of MIM, chromatin immunoprecipitation (ChIP) was carried out using an anti-FLAG antibody in MEK cells transfected with FLAG-MEF2C with or without fluid flow stimulation. FLAG-MEF2C co-immunoprecipitated with a MIM promoter fragment containing a putative MEF2C binding site both in the absence or presence of fluid flow stimulation (FIG. 3A). Chromatin IP using an anti-FLAG antibody also showed binding of FLAG-HDAC5 to the same promoter fragment in FLAG-HDAC5-transfected MEK cells (FIG. 3A).

To determine if MEF2C is important for MIM expression, MEF2C siRNA was introduced into wild-type MEK cells. The MEF2C siRNA knocked down MEF2C expression, as determined by qPCR, to 38% of that in cells transfected with a control siRNA, and MIM expression was reduced to 40% of that in the control cells (FIG. 3B). To test if MEF2C can indeed activate a MIM promoter, a 2 kb promoter fragment of MIM was cloned upstream of the luciferase reporter gene. The luciferase reporter construct was introduced into Pkd1^{-/-} MEK cells, which showed a drastically reduced level of endogenous MEF2C and MIM expression compared to those in Pkd1^{+/+} cells, as shown by microarray analysis and qPCR (FIGS. 13B-13C). Transfection of a MEF2C-expressing plasmid into the Pkd1^{-/-} cells only minimally stimulated MIM reporter expression. However, MEF2 members have been shown to function together with cell-type specific GATA transcription factors, which recruit MEF2 proteins to promoters through direct interactions between these proteins (Morin et al., 2000). A candidate for this cofactor is GATA6, also a flow-induced gene, that showed reduced expression in Pkd1^{-/-} MEK cells (Table 2 and FIGS. 13B-13C). Several GATA sites are present within 500 base pairs of the MEF2 binding motif in the MIM promoter. Co-transfection of Pkd1^{-/-} cells with both MEF2C and GATA6, but not either construct alone, stimulated MIM reporter expression by 3 fold (FIG. 3C). Moreover, overexpression of the non-phosphorylatable HDAC5 (HDAC5^{S250/489A}-GFP) in Pkd1^{+/+} cells strongly repressed MIM expression (FIG. 3D). The results set forth above suggest that MEF2C and HDAC5 directly regulate MIM expression in MEK cells.

### Example 4

### Mef2C and MIM Gene Disruption in Mouse Leads to Renal Tubule Dilation and Cyst Formation

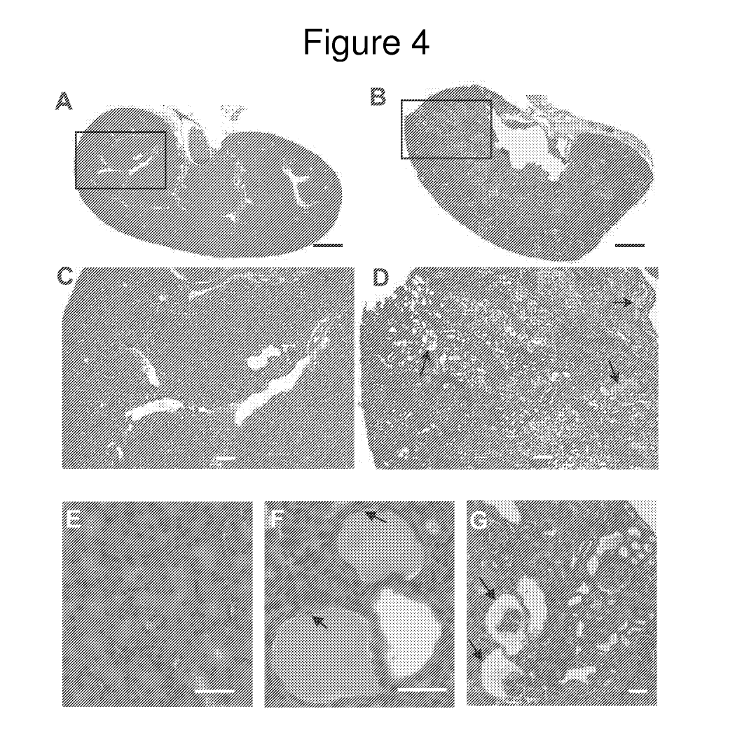

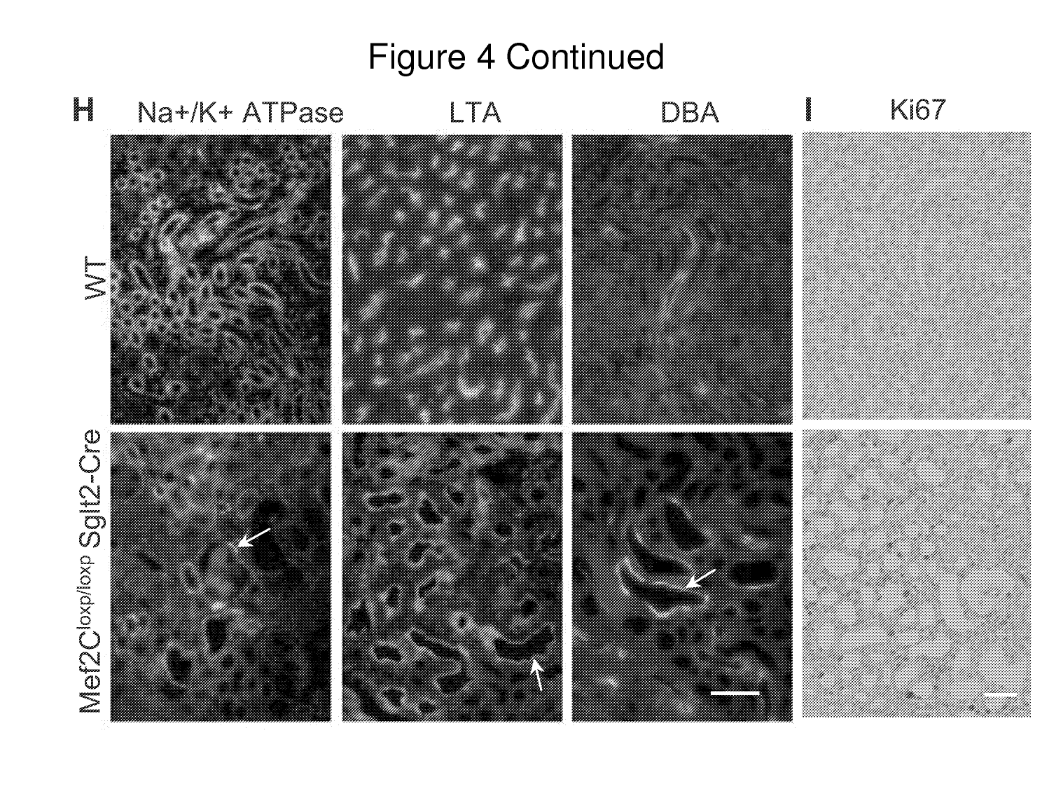

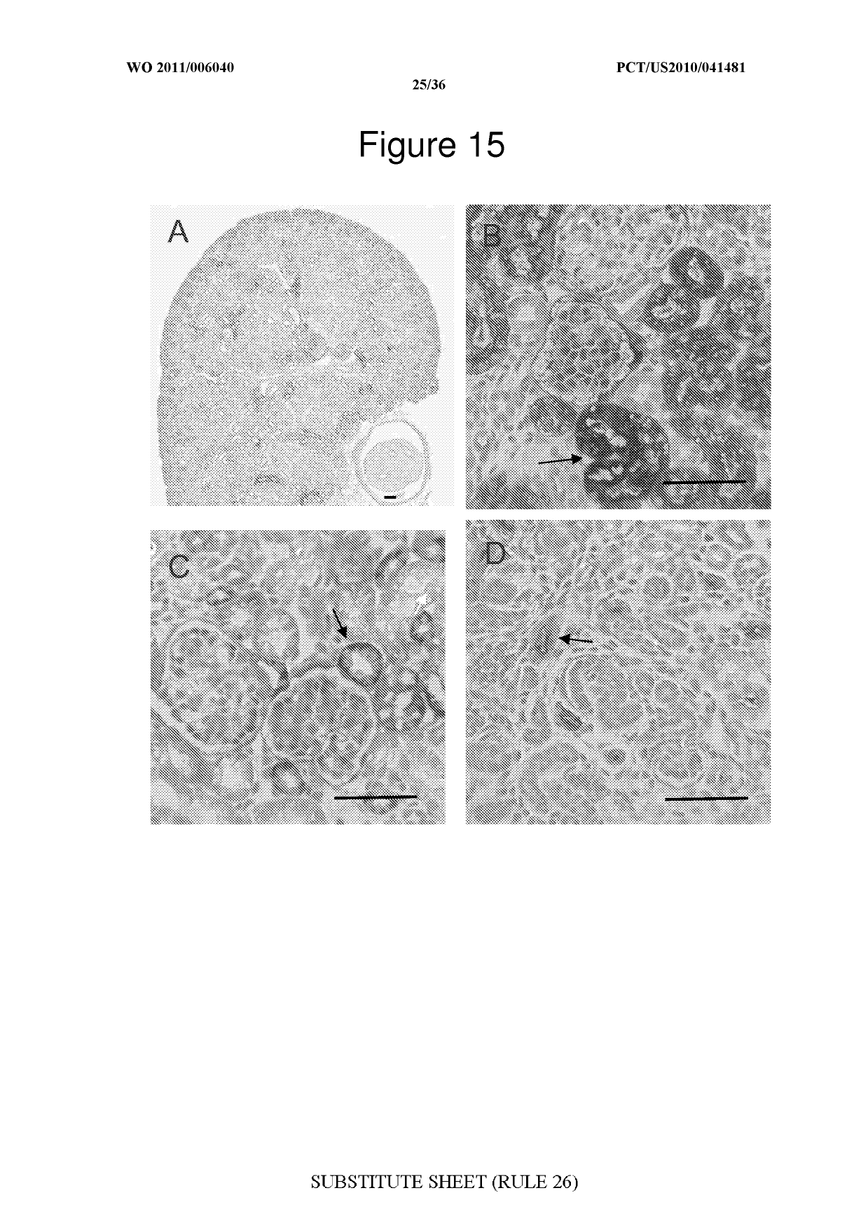

To determine if MEF2C-based transcription is important for kidney epithelial organization and proliferation, a previously established Mef2C conditional knock-out mouse line was obtained (Vong et al., 2005), because conventional Mef2C knockout resulted in early embryonic lethality (Lin et al., 1997). Kidney-specific disruption of Mef2C was accomplished using a SgIt2 promoter-driven Cre (Rubera et al., 2004), which is expressed in renal tubules and glomeruli (FIG. 15). In young Mef2C_{Ioxp/IoxP} SgIt2-Cre mutant mice (up to 2-months old), histological examination of the kidneys revealed no obvious abnormality (n=20); however, in mice 5 months or older, renal abnormalities were clearly observed in 9 out of 12 mice, including broadly distributed dilated tubules (FIGS. 4B and 4D, compared to FIGS. 4A and 4C), and small bi-lateral cysts with flat lining cells (FIG. 4F, compared to FIG. 4E). Marker staining showed that dilated tubules originated from distal tubules, proximal tubules and collecting ducts (FIG. 4H). Small glomerular cysts were also observed (FIG. 4G). Dilated tubules or cysts were associated with increased cell proliferation as indicated by Ki67 staining (FIG. 4I). These defects were not observed in kidneys of wild-type adult mice of the same age. This result indicates that MEF2C is important for the maintenance of normal tissue organization and repression of cell proliferation in differentiated kidney epithelia.

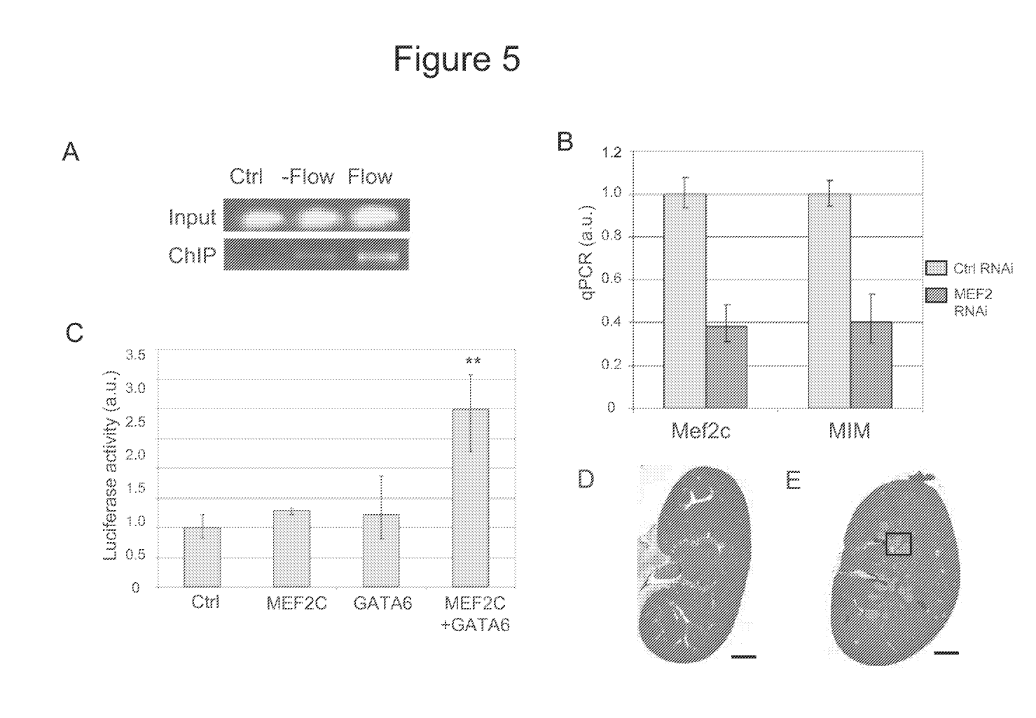

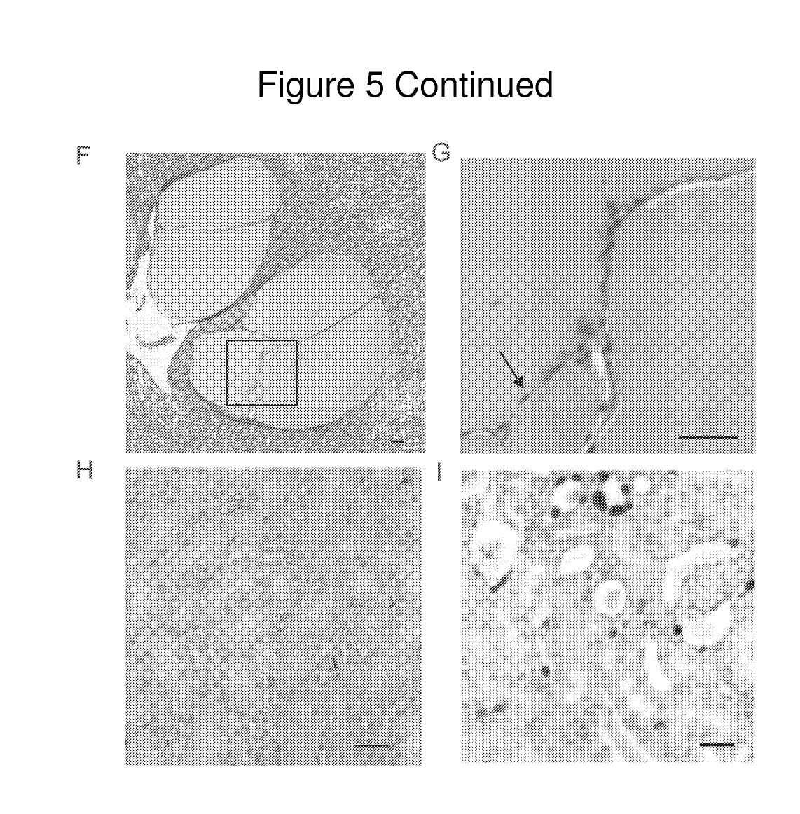

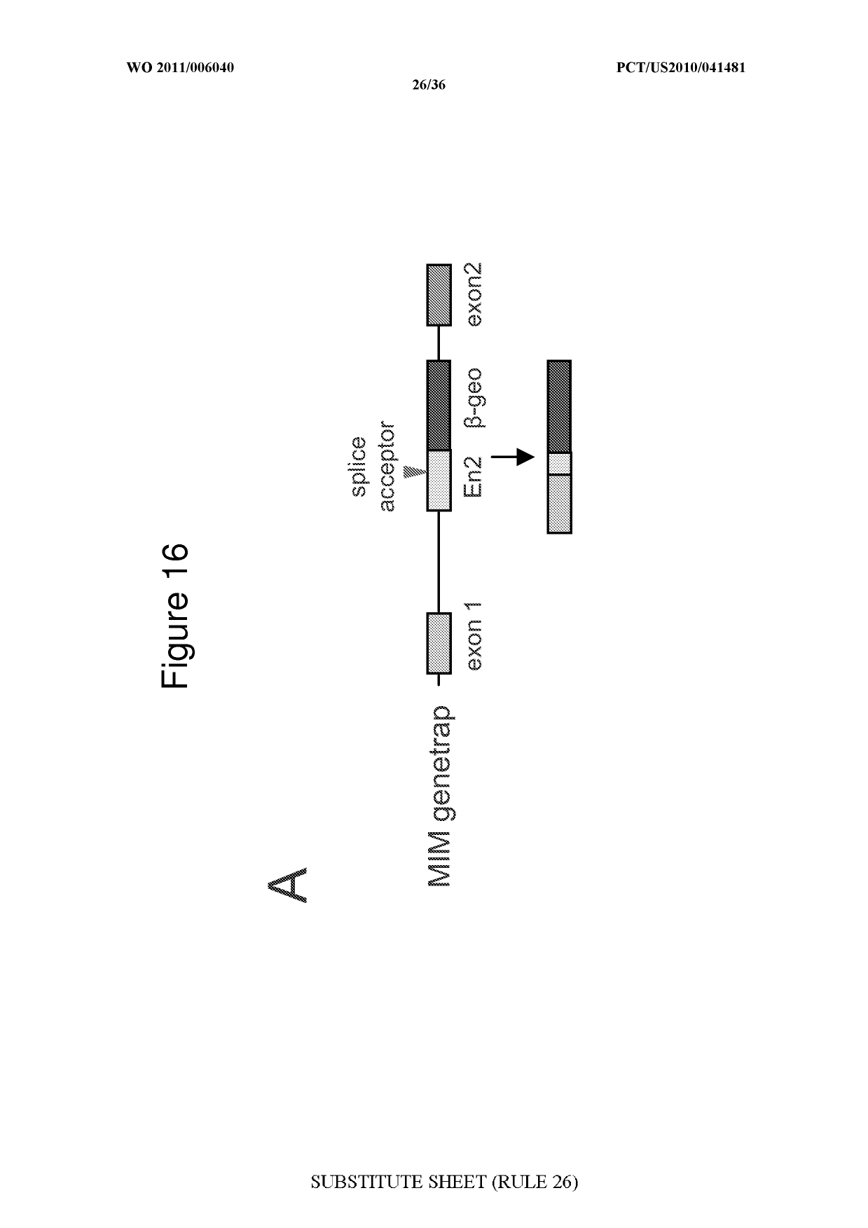

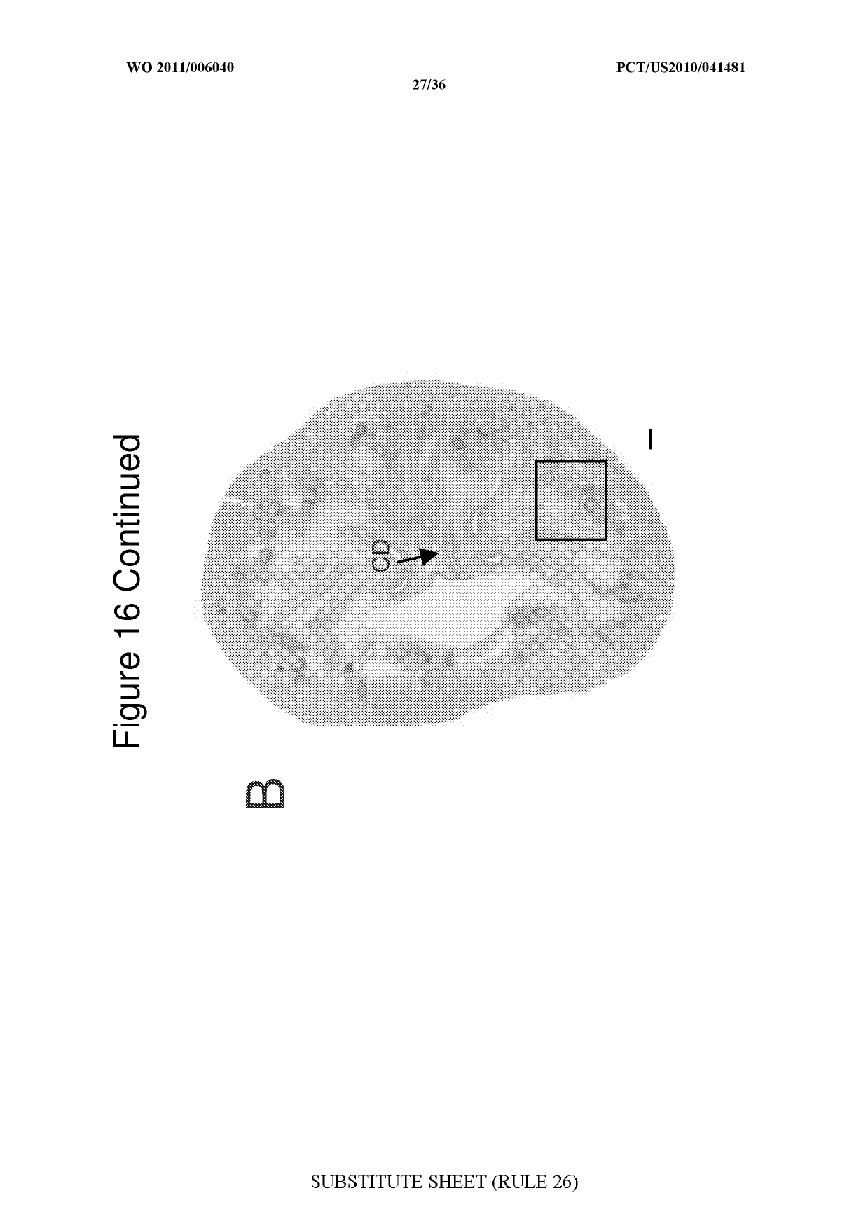

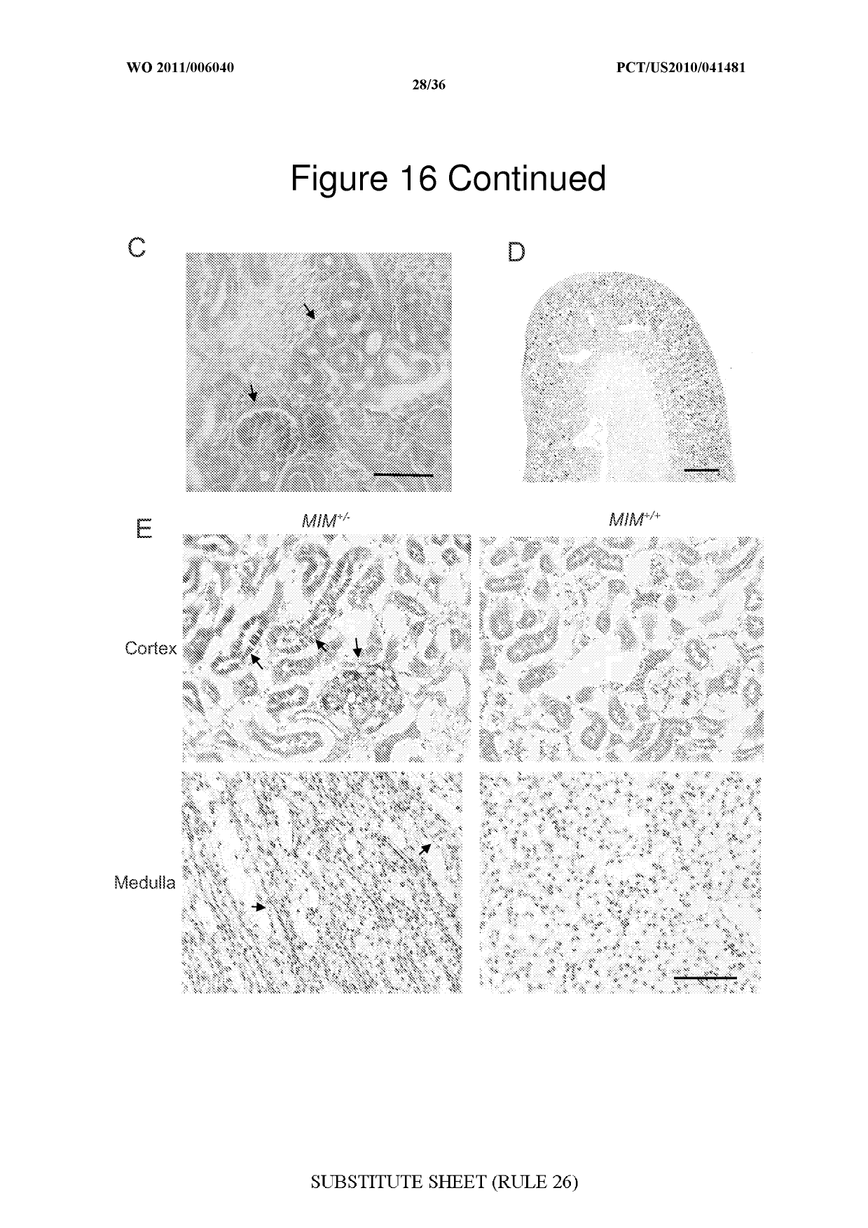

To assess the in vivo function of MIM, a MIM genetrap embryonic stem cell line was used to generate MIM-deficient mice (FIG. 16A). The genetrap was inserted at intron 1, truncating MIM after exon 1. X-gal staining of MIM^{+/-} embryos showed that MIM is expressed abundantly in embryonic heart, spinal cord, liver and the limb bud (data not shown). In E16.5 embryonic kidneys, MIM is expressed in branching collecting ducts, tubules and glomeruli (FIGS. 16B-16C). After birth, MIM is significantly expressed in renal cortex and weakly expressed in renal medula (FIGS. 16D-16E). MIM^{-/-} animals appeared normal after birth, but by 5 months, kidneys in 50% of the mutant animals (n=40) exhibited multiple large cysts (FIGS. 5E-5F, compared to FIG. 2A) with flat lining cells (FIG. 5G). As in Mef2C mutant kidneys, Ki67 staining showed drastically elevated proliferating cells (FIG. 5F, compared to 51). The above results suggest that MEF2C and its transcriptional targets, such as MIM, are required for preventing hyper-proliferation and cystogenesis in renal tubules.

### Example 5

### HDAC5 Inhibition Suppresses Renal Cyst Formation in Pkd2^{-/-} Mouse Embryos

A prediction based on a pathway where the calcium signal generated by polycytins deactivates HDAC5 is that loss-of-function mutations in HDAC5 should alleviate cyst formation in Pkd2^{-/-} mice. This possibility was tested by crossing pairs of Pkd2^{+/-} Hdac5^{+/-} double mutant mice. Pkd2^{-/-} embryos were known to die before or immediately after birth with many large renal cysts (Wu et al., 2000). Genotyping of 89 E18.5 embryos from 11 crosses showed that, for reasons unknown, no Pkd2^{-/-} Hdac5^{+/+} and only 1 Pkd2^{-/-} Hdac5^{-/-} embryo was observed from these crosses (expected and observed frequencies of all genotypes are listed in Table 3 below).

TABLE 3
Quantification of genotypes observed from 89 viable E18.5 embryos
from 11 crosses of Pkd2^{+/-} Hdac5^{+/-} male and female mice.
The numbers shown below are percentages (%).

| | Pkd2 | | | | | | | | |
|---|---|---|---|---|---|---|---|---|---|
| | −/− | −/− | −/− | +/− | +/− | +/− | +/+ | +/+ | +/+ |
| | Hdac5 | | | | | | | | |
| | −/− | +/− | +/+ | −/− | +/− | +/+ | −/− | +/− | +/+ |
| Ob- | 1.1 | 4.5 | 0 | 13.5 | 29.2 | 9.0 | 18.0 | 22.5 | 2.2 |
| served | | | | | | | | | |
| fre- | | | | | | | | | |
| quency | | | | | | | | | |
| Ex- | 6.25 | 12.5 | 6.25 | 12.5 | 25 | 12.5 | 6.25 | 12.5 | 6.25 |
| pected | | | | | | | | | |
| fre- | | | | | | | | | |
| quency | | | | | | | | | |

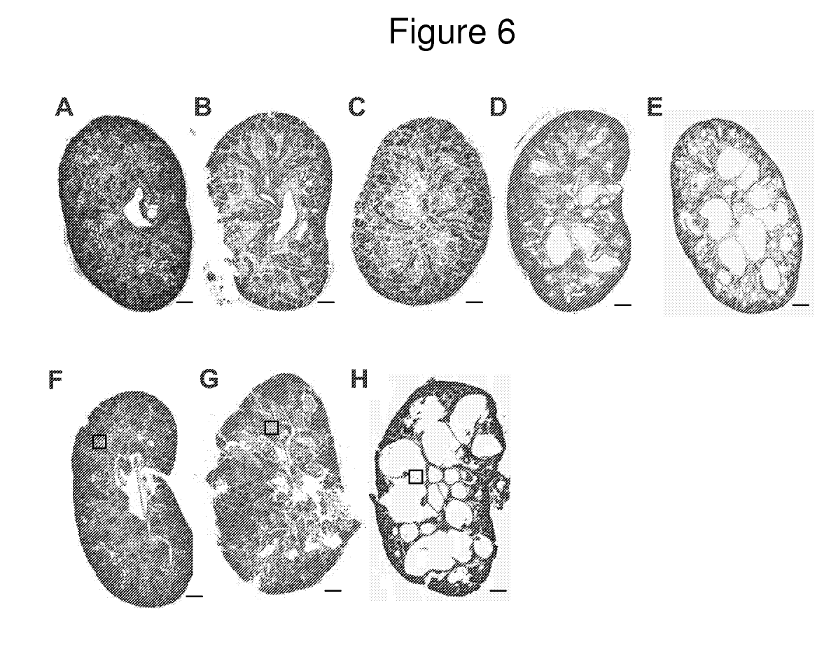

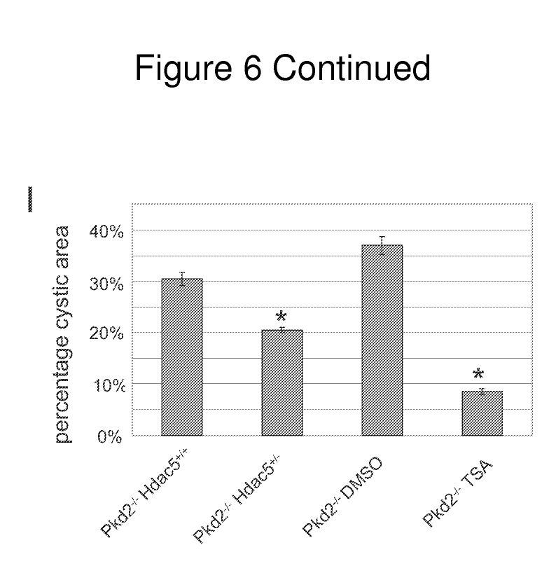

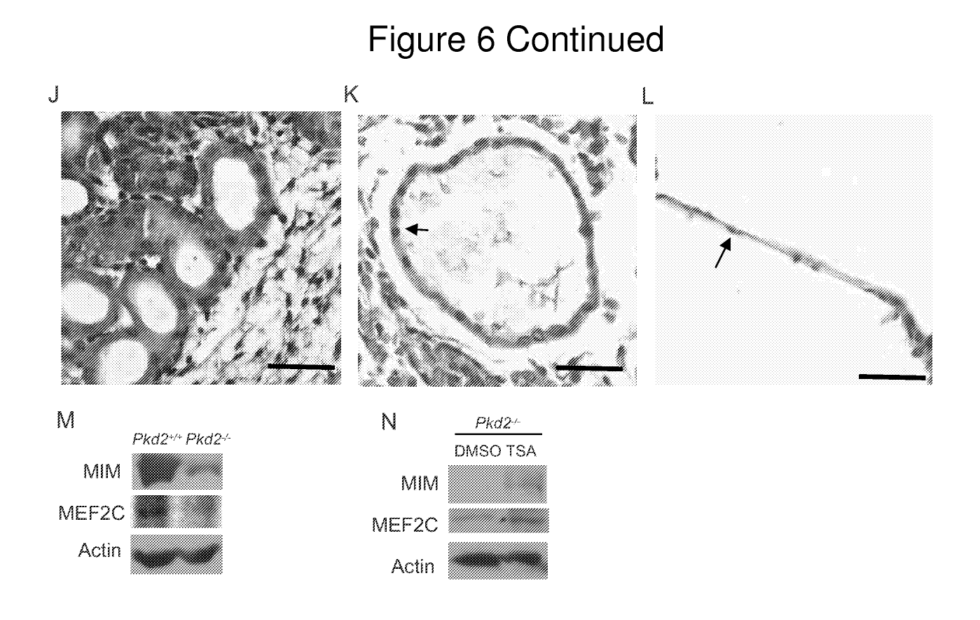

However, kidneys of Pkd2^{-/-} Hdac5^{+/-} embryos (n=7) exhibited visibly reduced cyst formation, compared to Pkd2^{-/-} Hdac5^{+/+} kidneys from embryos of Pkd2^{+/-} Hdac5^{+/+} parents of the same genetic background (See, FIG. 6, comparing 6D with 6E). Quantification of cyst area percentages further confirmed significantly reduced cyst formation in Pkd2^{-/-} Hdac5^{+/-} kidneys compared to that in Pkd2^{-/-} Hdac5^{+/+} kidneys (FIG. 6I).

To test further the possibility that reducing the activity of HDAC5 could suppress cyst formation in Pkd2^{-/-} embryos, trichostatin A (TSA), a chemical inhibitor against type I and II HDACs (Drummond et al., 2005), was injected into pregnant Pkd2+/− female mice from 10.5 dpc after mating with Pkd2+/− males, and embryonic kidneys were analyzed at 18.5 dpc. In all Pkd2^{-/-} embryos (n=7) from TSA-injected mothers, kidney cyst formation was drastically reduced compared to those from control DMSO-injected mothers (n=6) (compare FIGS. 6G with 6H, and FIG. 6I). Even in the much smaller cysts that persisted in the Pkd2^{-/-} kidneys from TSA-treated mothers, cyst lining cells exhibited a more normal cuboidal morphology as opposed to the flat morphology of Pkd2^{-/-} cyst lining cells (FIGS. 6J-L). Apart from the above effect on cystic kidneys, TSA injection did not affect the morphology of renal tubules in Pkd2^{+/+} embryonic kidneys (FIGS. 6F and 6J). Immunoblot analysis showed that MIM and MEF2C expression levels were respectively 10 fold and 3 fold reduced in E18.5 Pkd2^{-/-} embryonic kidneys compared with those in Pkd2^{+/+} embryonic kidneys (FIG. 6N). Pkd2^{-/-} embryonic kidneys from TSA-treated mothers showed a 4-fold enhancement of expression of MEF2C and a drastically increased expression of MIM compared with those in Pkd2^{-/-} kidneys from DMSO-treated mothers (FIG. 6N), suggesting that TSA stimulated the expression of MEF2C target genes.

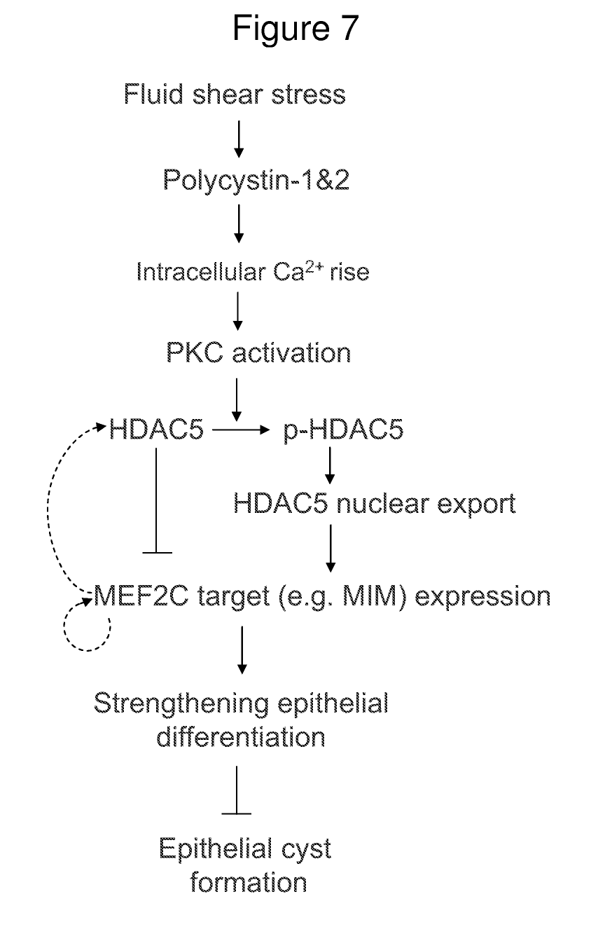

The results of this study have revealed an unexpected role for HDAC5 and MEF2C as targets of polycystins' mechanosensory function in renal epithelial cells. In this pathway, laminar fluid flow across the apical surface of epithelial cells induces phosphorylation and nuclear export of HDAC5 in a polycytin-1 and calcium-dependent manner, leading to activation of MEF2C-based transcription (summarized in FIG. 7). Conditional disruption of MEF2C or genetrap-based inactivation of MIM, one of MEF2C's transcriptional targets, led to renal tubule dilation and epithelial cysts in adult mice by 5 months of age, although the number and size of the cysts were not as severe as those in animals with conditional knockout or somatic inactivation of polycystins (Lantinga-van Leeuwen et al., 2007; Piontek et al., 2007; Wu et al., 2000). Interestingly, it also took five months for cysts to occur after inactivation of Pkd-1 at postnatal day 14 (Piontek et al., 2007). These findings support the idea that there may be a developmental factor that contributes to the onset of cystogenesis in adult kidneys.

Cardiac hypertrophy in the adult heart is an adaptive mechanism of activation of an embryonic program to improve cardiac pump function in response to an increased workload or mechanical stress (Xu et al., 2006). The similarity between the cardiac hypertrophy pathway and the response to fluid flow in renal epithelial cells raises a question as to whether cystogenesis in ADPKD could in part be an outcome of a lack of the proper response to certain stress or an increase in workload on renal epithelial cells. The nephrons are frequently challenged with fluctuations in fluid shear stress and osmotic pressure, and these changes may be enhanced during certain developmental stages or under certain dietary, hormonal or pathological conditions. Renal hypertrophy is a well known phenomenon that occurs after nephretomy or under other stress conditions (Hostetter, 1995). A recent study reported that treatment of a PCK rat (a recessive PKD model) with 1-deamino-8-D-arginine vasopressin (dDAVP), an agonist of the Arginine vasopressin V2 receptor, led to aggravated cystic disease, but the same treatment on wild-type rats resulted in renal hypertrophy (Wang et al., 2008). This observation and the finding that polycystins regulate a molecular pathway known to be involved in cardiac hypertrophy raise a question as to whether polycystins function at the crossroads between hypertrophy and hyperplasia in renal epithelial cells in response to stress or certain physiological stimuli.

MEF2C-based transcriptional activation leads to increased expression of contractile proteins and metabolic enzymes in cardiac tissues. If there is indeed a similar hypertrophic pathway in the kidney, MEF2C may cooperate with other tissue specific transcription factors, such as GATA6, to control the expression of genes involved in strengthening the differentiated state of renal epithelial tubules in response to stress. Consistent with this, MIM, a founding member of the family of IMD-containing actin binding proteins, as well as another member of this family, BAIAP2L1 (Table 2), were found to be a MEF2C target in epithelial cells. The IMD domain has the ability to induce membrane deformation and actin bundling in cultured cells (Machesky and Johnston, 2007). IMD proteins also possess a COON-terminal WASP homology-2 (WH2) domain, which binds monomeric actin, and thus may be involved in actin polymerization. The actin cytoskeleton plays important roles in almost every aspect of epithelial cell and tissue organization (Leiser and Molitoris, 1993). Actin structures built through MIM and BAIAP2L1 may serve to strengthen epithelial cell polarity or cell-cell and cell-matrix interactions within epithelial tissues. Interestingly, several actin-interacting proteins (α-actinin, tropomyosin, troponin, mDia1, etc) were found to bind polycystins and some were shown to modulate PC-2 channel activity (Li et al., 2003a; Li et al., 2005a; Li et al., 2003b; Rundle et al., 2004). A recent study reported that polycystins regulate pressure sensing through stretch-activated channels in myocytes, and this function requires an intact actin cytoskeleton and the actin-binding protein filamin (Sharif-Naeini et al., 2009). Therefore, it is also possible that the actin-regulatory activities of MIM and BAIAP2L1 are in turn involved in polycystin-mediated signaling, with the membrane-bending activity of IMD domains directly modulating the tension in the plasma membrane. The function of MIM may extend beyond cytoskeletal regulation, however, as in MIM^{-/-} mouse kidneys, hyperproliferation was evident in renal tubules. Also known as basal cell carcinoma-enriched gene 4 (BEG4), MIM was previously shown to be a target and modulator of Sonic hedgehog (Shh) signaling in the skin (Callahan et al., 2004). A role in Shh signaling could underlie increased cell proliferation in MIM^{-/-} kidneys.

While MEF2C was the only MEF2 member whose expression level was found to increase in response to fluid flow in the microarray analysis, MEF2A, which functions in parallel with MEF2C in regulating cardiac hypertrophy (Xu et al., 2006), is also expressed in MEK cells (data not shown). The functional redundancy of MEF2C and MEF2A (Piontek et al., 2007) may account for the much milder cystic phenotype in Mef2C knockout kidneys than in polycystin knockout mice. Alternatively this may be explained by the fact that SgIt2-Cre was not expressed in all renal tubules (FIG. 15) and that MEF2C activation is likely to be one of several downstream pathways regulated by polycystins and important for the maintenance of epithelial architecture. Although the reduction in cyst severity in Pkd2^{-/-} embryonic kidneys caused by Hdac5 heterozygosity is consistent with the proposed epistatic relationship between Pkd2 and Hdac5, this result does not rule out potential regulation by other members of the class II HDAC family. Furthermore, it is known that Class IIa HDACs, such as HDAC5, lack intrinsic enzymatic activity and require complex formation with HDAC3, a class I HDAC that is sensitive to TSA, for their transcriptional repression activity (Fischle et al., 2002). The restoration of MIM expression level in TSA-treated Pkd2^{-/-} embryonic kidneys is consistent with stimulation of MEF2C target gene expression through functional inhibition of HDAC5 or other class IIa HDACs. This may tip the balance toward up-regulation of MEF2-downstream target genes (e.g., MIM), leading to reinforcement of epithelial differentiation and suppression of cell proliferation. The anti-proliferative effect of HDAC inhibitors is in fact the basis for clinical studies of these molecules in anti-cancer therapies (Johnstone, 2002). Accordingly, HDAC inhibitors may be explored as therapeutic agents for the treatment of ADPKD.

### Example 6

### HDAC5 Inhibitor Suppressed Cyst Formation in cAMP-Induced Cysts in Mouse Embryonic Kidney Organ Cultures

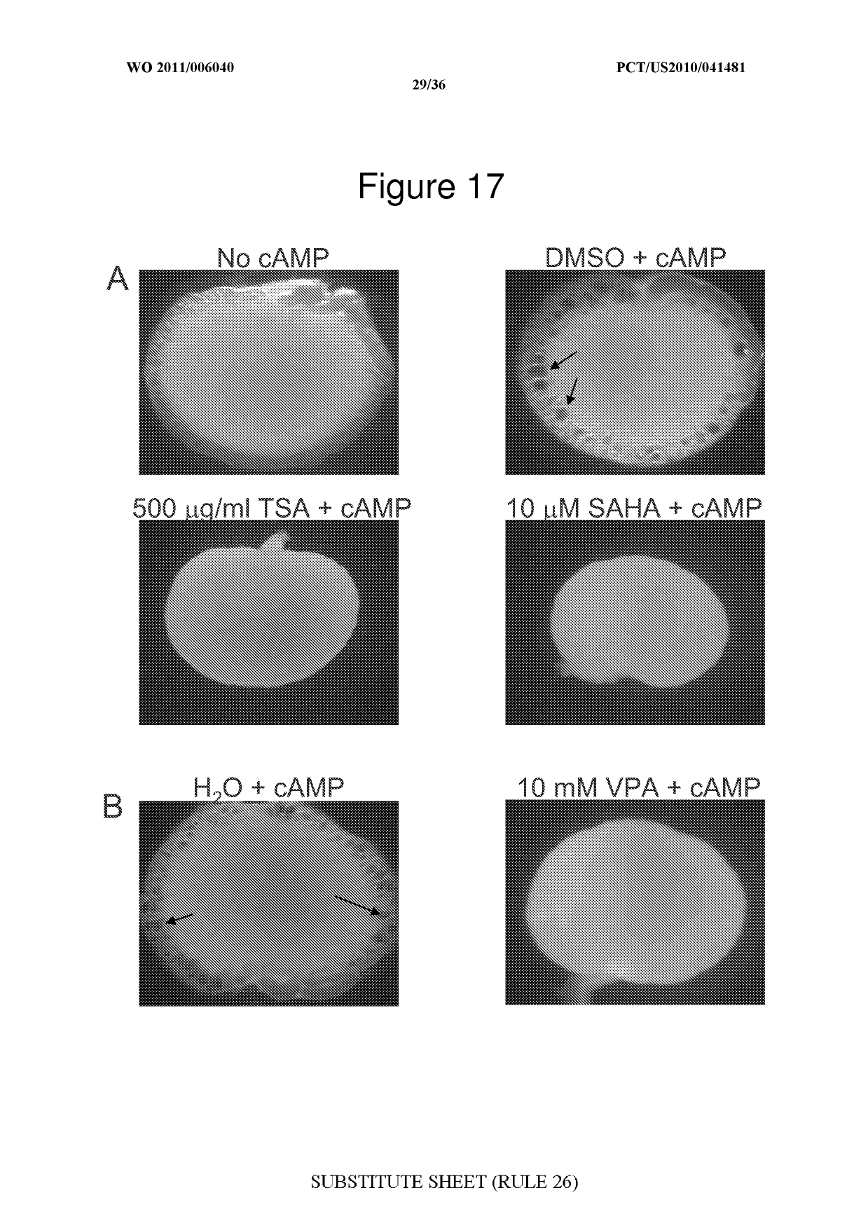

Embryonic kidneys were dissected from embryos of C57BL/6 at E15.5 in phosphate buffered saline (PBS), with calcium and magnesium, plus penicillin-streptomycin-glutamine (Invitrogen Corp., Carlsbad, Calif.). The dissected kidneys were cultured at 37° C. in DMEM/F12 media containing 2 mM L-glutamine, 10 mM HEPES (Invitrogen Corp.), 5 mg/ml insulin, 5 mg/ml transferrin, 2.8 mM selenium, 25 ng/ml prostaglandin E1, 32 pg/ml 2,3,5-triido-L-thyronine (T3) and 250 U/ml penicillin-streptomycin. The kidneys were cultured with or without the HDAC inhibitors in the concentrations shown in FIG. 17 for 48 hours. 50 mM 8-bromo-cAMP (Sigma, St. Louis, Mo.) were then added to the culture medium. The culture was allowed to continue for 5 days. The cultured kidneys were then fixed with 4% paraformaldehyde in PBS for 6 hours, washed with PBS twice for 5 minutes each, and then transferred to 70% ethanol for short-term storage at room temperature or for more extended storage at 4° C. The fixed kidney samples were prepared for hematoxylin and eosin staining and lectin staining following histology protocols known in the art.

In FIG. 17A, E15.5 embryonic kidneys were isolated from pregnant C57BL/6 mice and cultured in vitro for 5 days. Kidneys were treated with DMSO and 100 μM cAMP to induce cyst formation or cAMP with various concentrations of TSA or suberoylanilide hydroxamic acid (SAHA) as labeled for the duration of the culture. In FIG. 17B, E15.5 embryonic kidneys were cultured in vitro and treated with H₂O and cAMP or valproic acid (VPA) and cAMP for 5 days. Arrows point to cAMP-induced cortical cysts. These results show that HDAC inhibitors inhibit cAMP-induced cysts in mouse embryonic kidney organ cultures.

### Example 7

### HDAC5 Expression is Decreased in Human ADPKD Cyst Lining Cells

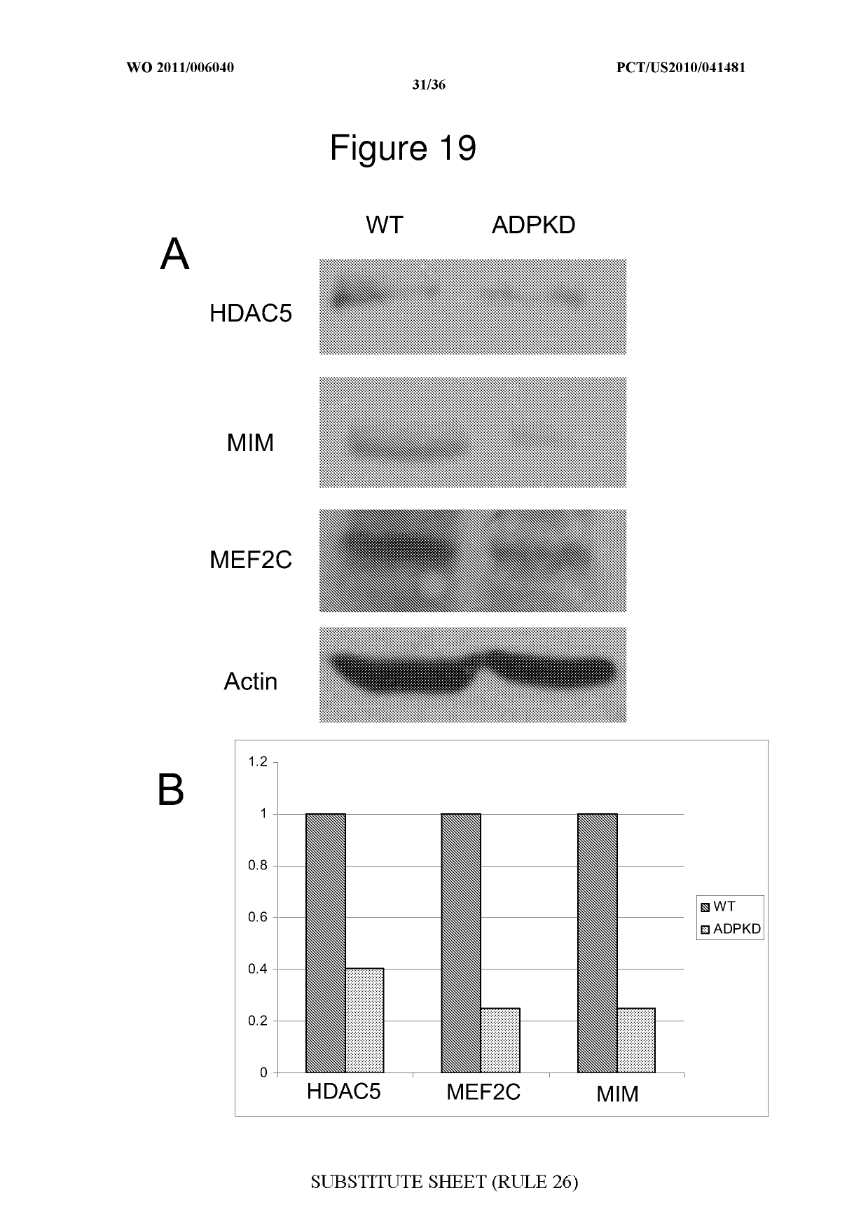

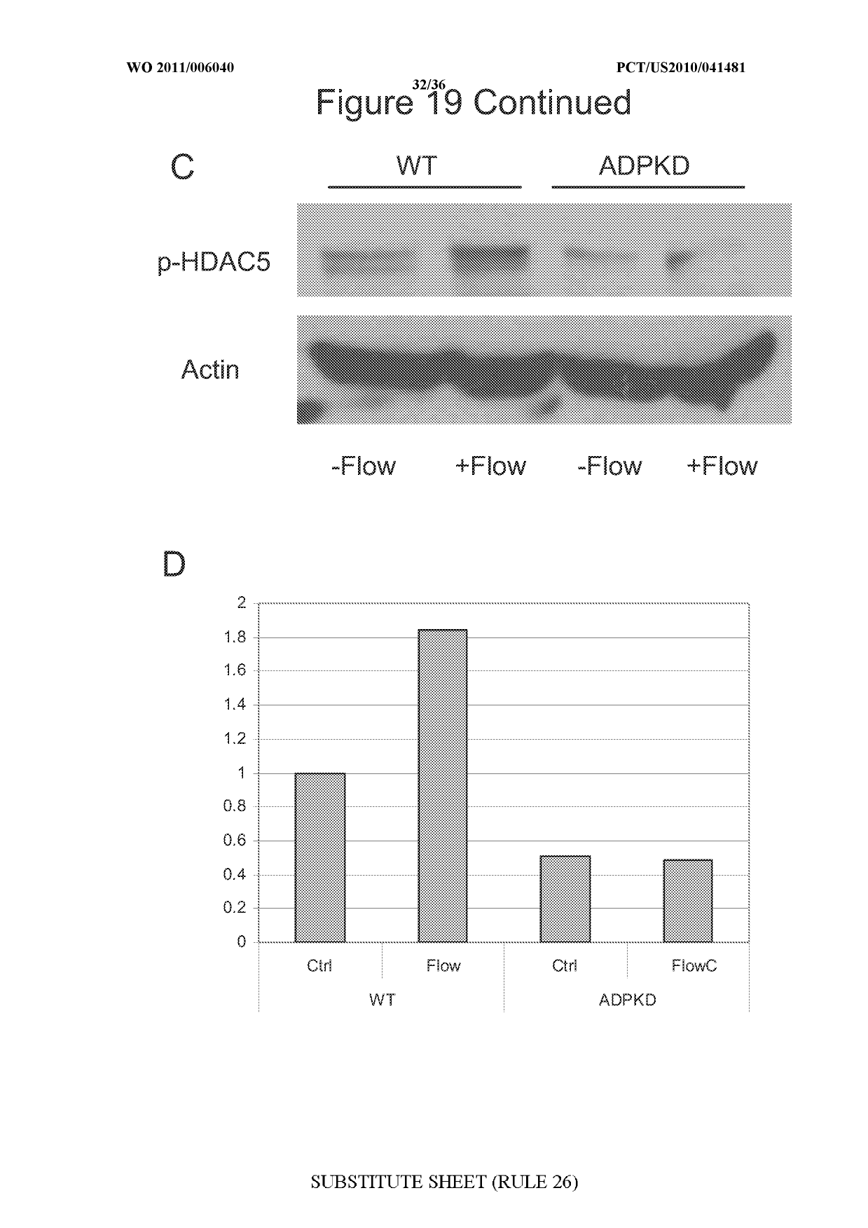

Human kidney primary cells were cultured for 4 days in 1% FBS DMEM/F12 (Invitrogen) with Insulin-Transferrin-Selenium-X. The expression levels of HDAC5, MEF2C and MIM were then analyzed in these cells by immunoblot analysis. The results show that the expression levels of HDAC5, MEF2C and MIM decreased in human ADPKD cyst lining cells compared to normal human kidney epithelial cells (FIGS. 19A and B).

To determine the effects of fluid flow on phosphorylation of HDAC5, fluid (culture media) flow was applied at 0.2 ml/min using a peristaltic pump across each well of 6-well plates, with tubings connected through two 18 Gauge needles at opposing sides of the well, for four hours prior to immunoblot analysis. In this experiment, a phosphor-specific antibody against phosphorylated serine 489 (HDAC5 (Phospho-Ser498 antibody) (Signalway Antibody) was used. Under these conditions, fluid flow-stimulated phosphorylation of HDAC5 occurred in normal human kidney epithelial cells but not ADPKD cyst lining cells (FIGS. 19C and D).

### Example 8

### HDAC5 Inhibitors Improve Kidney Function in a Mouse Model of PKD

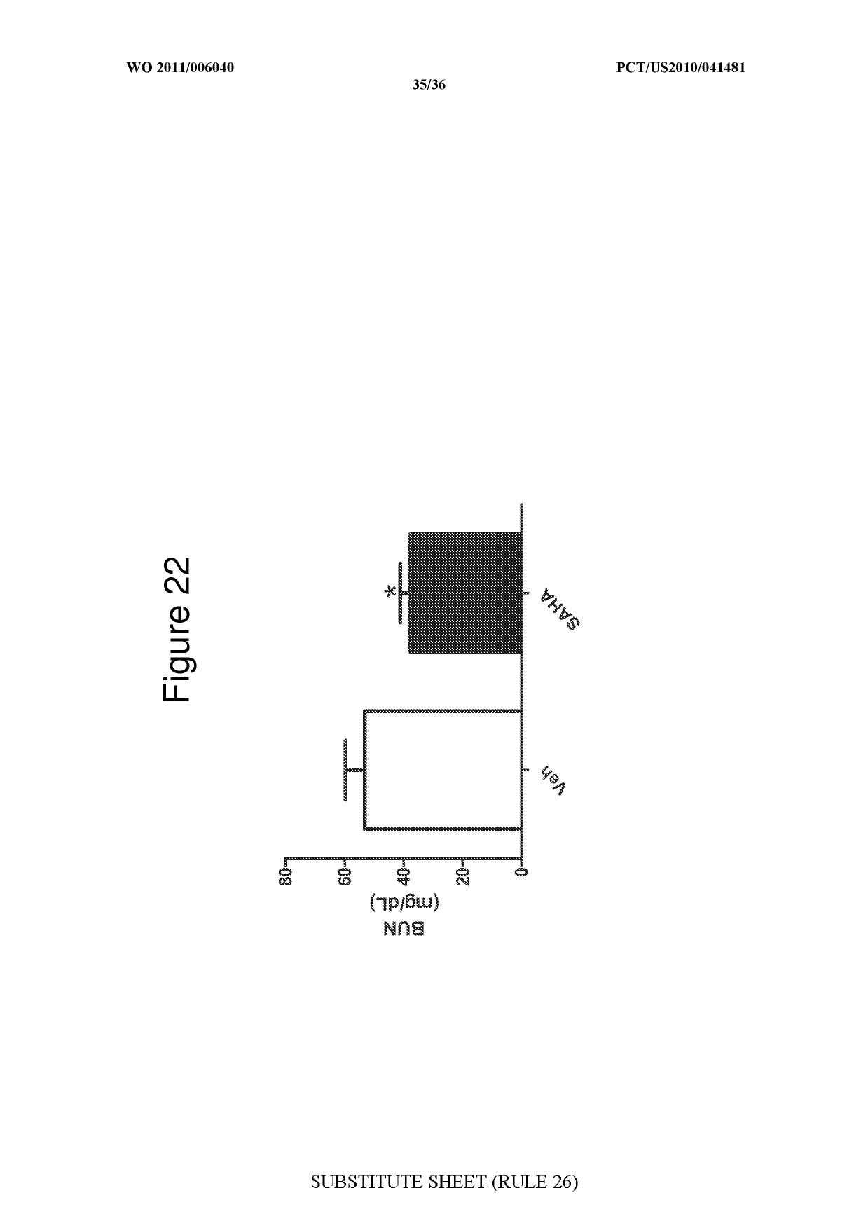

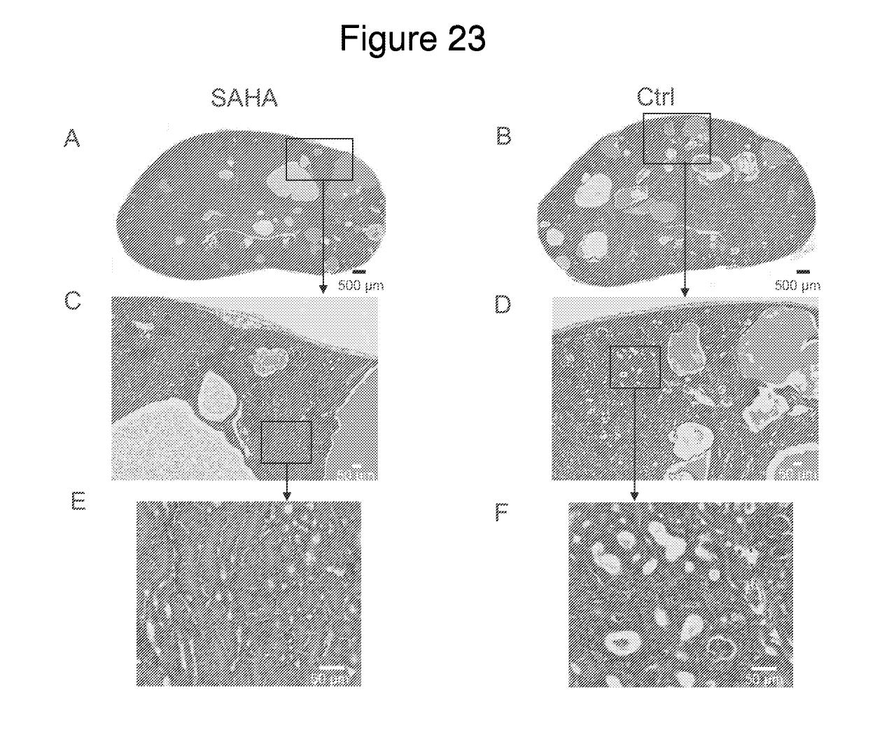

PKD^{ws25/-} mice (Wu et al., 1998) were obtained from Albert Einstein College of Medicine (Bronx, N.Y.). PKD^{ws25/-} mice escape the embryonic lethality of the PKD^{-/-} phenotype, but recapitulate the human ADPKD phenotype in that they develop cysts early in life (Wu et al., 1998). Eight week old PKD^{ws25/-} mice (n=15 per group) were treated with 100 mg/kg vorinostat (SAHA) or vehicle (Veh). Animals were dosed daily by i.p. for 5 days followed by 2 days off, for a total of 4 weeks. Both treatments were well tolerated, one animal was lost from the vorinostat group. At the end of study, animals were sacrificed and blood taken for analysis of blood urea nitrogen (BUN) levels using the manufacturer's protocol for Hitachi Urea Nitrogen (BUN) Reagent Set (Pointe Scientific, Inc., Canton, Mich.). Animals treated with SAHA had significantly lower BUN levels, compared to vehicle treated animals, indicating improved kidney function (FIG. 22). Additionally, small cysts or tubule dilations were widely observed in the control images whereas SAHA-treated images showed well organized, nearly normal epithelial morphology (FIG. 23). These experiments indicate that SAHA improves kidney function in a mouse model of PKD.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### CITED DOCUMENTS

Patents, patent applications, publications, procedures, and the like are cited throughout this application and are cited below, the disclosures of which are incorporated herein by reference in their entireties.
Azzam, R., Chen, S. L., Shou, W., Mah, A. S., Alexandru, G., Nasmyth, K., Annan, R. S., Carr, S. A. and Deshaies, R. J. (2004). Phosphorylation by cyclin B-Cdk underlies release of mitotic exit activator Cdc14 from the nucleolus. Science 305, 516-519.
Battini, L., Macip, S., Fedorova, E., Dikman, S., Somlo, S., Montagna, C. and Gusella, G. L. (2008). Loss of polycystin-1 causes centrosome amplification and genomic instability. Hum Mol Genet. 17, 2819-2833.
Berbari, N. F., O'Connor, A. K., Haycraft, C. J. and Yoder, B., K. (2009). The primary cilium as a complex signaling center. Curr Biol. 19, R526-535.
Bhunia, A. K., Piontek, K., Boletta, A., Liu, L., Qian, F., Xu, P. N., Germino, F. J. and Germino, G. G. (2002). PKD1 induces p21(waf1) and regulation of the cell cycle via direct activation of the JAK-STAT signaling pathway in a process requiring PKD2. Cell 109, 157-168.
Boletta, A. and Germino, G. G. (2003). Role of polycystins in renal tubulogenesis. Trends Cell Biol. 19.
Bossuyt, J., elmstadter, H., Wu, X. and Clements-Jewery, H. (2008). Ca2+/Calmodulin-Dependent Protein Kinase II and Protein Kinase D Overexpression Reinforce the Histone Deacetylase 5 Redistribution in Heart Failure. Circulation Research 102, 695-702.
Callahan, C. A., Ofstad, T., Horng, L., Wang, J. K., Zhen, H. H., Coulombe, P. A. and Oro, A. E. (2004). MIM/BEG4, a Sonic hedgehog-responsive gene that potentiates Gli-dependent transcription. Genes Dev 18, 2724-2729.
Davenport, J. R., Watts, A. J., Roper, V. C., Croyle, M. J., van Groen, T., Wyss, J. M., Nagy, T. R., Kesterson, R. A. and Yoder, B. K. (2007). Disruption of intraflagellar transport in adult mice leads to obesity and slow-onset cystic kidney disease. Curr Biol. 17, 1586-1594.
Drummond, D., Noble, C., Kirpotin, D., Guo, Z., Scott, G. and Benz, C. (2005). Clinical development of histone deacetylase inhibitors as anticancer agents. Annu Rev Pharmacol Toxicol 45, 495-528.
Eley, L., Yates, L. M. and Goodship, J. A. (2005). Cilia and disease. Curr Opin Genet Dev. 15, 308-314.
Fischle, W., Dequiedt, F., Hendzel, M. J., Guenther, M. G., Lazar, M. A., Voelter, W. and Verdin, E. (2002). Enzymatic activity associated with class II HDACs is dependent on a multiprotein complex containing HDAC3 and SMRT/N—CoR. Mol. Cell. 9, 45-57.
Gattone, V. H., Chen, N. X., Sinders, R. M., Seifert, M. F., Duan, D., Martin, D., Henley, C. and Moe, S. M. (2009). Calcimimetic inhibits late-stage cyst growth in ADPKD. J Am Soc Nephrol. 20, 1527-1532.
Grantham, J. J. (2003). Lillian Jean Kaplan International Prize for advancement in the understanding of polycystic kidney disease. Understanding polycystic kidney disease: a systems biology approach. Kidney Int. 64.
Grimm, D. H., Praetorius, H. A. and Cai, Y. (2002). A role for polycystin-2 in the cilium mechanostimulation pathway. J Am Soc Nephrol 13, F541-552.
Haberland, M., Arnold, M. A., McAnally, J., Phan, D., Kim, Y. and Olson, E. N. (2007). Regulation of HDAC9 gene expression by MEF2 establishes a negative-feedback loop in the transcriptional circuitry of muscle differentiation. Mol Cell Biol 27, 518-525.
Hostetter, T. H. (1995). Progression of renal disease and renal hypertrophy. Annu Rev Physiol. 57.
Ibraghimov-Beskrovnaya, O. et al., Hum. Mol. Genet. 9, 1641 (Jul. 1, 2000).
Irizarry, R. A., Hobbs, B., Collin, F., Beazer-Barclay, Y. D., Antonellis, K. J., Scherf, U. and Speed, T. P. (2003). Exploration, normalization, and summaries of high density oligonucleotide array probe level data. Biostatistics 4, 249-264.
Johnstone, R. W. (2002). Histone-deacetylase inhibitors: novel drugs for the treatment of cancer. Nat Rev Drug Discov 1, 287-299.
Köttgen, M., Buchholz, B., Garcia-Gonzalez, M. A., Kotsis, F., Fu, X., Doerken, M., Boehlke, C., Steffl, D., Tauber, R., Wegierski, T. et al. (2008). TRPP2 and TRPV4 form a polymodal sensory channel complex. J. Cell Biol. 182, 437-447.
Lantinga-van Leeuwen, I. S., Leonhard, W. N., van der Wal, A., Breuning, M. H., de Heer, E. and Peters, D. J. (2007). Kidney-specific inactivation of the Pkd1 gene induces rapid cyst formation in developing kidneys and a slow onset of disease in adult mice. Hum Mol Genet. 16, 3188-3196.
Leiser, J. and Molitoris, B. A. (1993). Disease processes in epithelia: the role of the actin cytoskeleton and altered surface membrane polarity Biochim Biophys Acta. 1225, 1-13.
Li, G., Vega, R., Nelms, K., Gekakis, N., Goodnow, C., McNamara, P., Wu, H., Hong, N. A. and Glynne, R. (2007). A role for Alström syndrome protein, alms1, in kidney ciliogenesis and cellular quiescence. 3 1, e8.
Li, Q., Dai, Y., Guo, L., Liu, Y., Hao, C., Wu, G., Basora, N., Michalak, M. and Chen, X. Z. (2003a). Polycystin-2 associates with tropomyosin-1, an actin microfilament component. J Mol Biol 325, 949-962.
Li, Q., Montalbetti, N., Shen, P. Y., Dai, X. Q., Cheeseman, C. I., Karpinski, E., Wu, G., Cantiello, H. F. and Chen, X. Z. (2005a). Alpha-actinin associates with polycystin-2 and regulates its channel activity. Hum Mol Genet. 14, 1587-1603.
Li, Q., Shen, P. Y., Wu, G. and Chen, X. Z. (2003b). Polycystin-2 interacts with troponin I, an angiogenesis inhibitor. Biochemistry 42, 450-457.
Li, X., Luo, Y., Starremans, P. G., McNamara, C. A., Pei, Y. and Zhou, J. (2005b). Polycystin-1 and polycystin-2 regulate the cell cycle through the helix-loop-helix inhibitor Id2. Nature cell biology 7, 1202-1212.
Lin, Q., Schwarz, J., Bucana, C. and Olson, E. N. (1997). Control of mouse cardiac morphogenesis and myogenesis by transcription factor MEF2C. Science 276, 1404-1407.
Machesky, L. M. and Johnston, S. A. (2007). MIM: a multifunctional scaffold protein. J Mol Med 85, 569-576.
Markoff, A. et al., J. Mol. Biol. 369, 954 (Jun. 15, 2007).
McKinsey, T. A., Zhang, C. L. and Olson, E. N. (2001). Control of muscle development by dueling HATs and HDACs Current Opinion in Genetics & Development 11, 497-504.
McKinsey, T. A., Zhang, C. L. and Olson, E. N. (2002). MEF2: a calcium-dependent regulator of cell division, differentiation and death. Trends Biochem Sci 27, 40-47.
McKinsey, T. A., Zhang, C. L., Lu, J. and Olson, E. N. (2000). Signal-dependent nuclear export of a histone deacetylase regulates muscle differentiation. Nature 408, 106-111.
Mochizuki, T., Wu, G., Hayashi, T., Xenophontos, S. L., Veldhuisen, B., Saris, J. J., Reynolds, D. M., Cai, Y., Gabow, P. A., Pierides, A. et al. (1996). PKD2, a gene for polycystic kidney disease that encodes an integral membrane protein. Science 272, 1339-1342.
Morin, S., Charron, F., Robitaille, L. and Nemer, M. (2000). GATA-dependent recruitment of MEF2 proteins to target promoters. Embo J 19, 2046-2055.
Nachury, M. V. et al., Cell 129, 1201 (Jun. 15, 2007).
Nagao, S., Nishii, K., Yoshihara, D., Kurahashi, H., Nagaoka, K., Yamashita, T., Takahashi, H., Yamaguchi, T., Calvet, J. P. and Wallace, D. P. (2008). Calcium channel inhibition accelerates polycystic kidney disease progression in the Cy/+ rat. Kidney Int. 73, 269.
Nagao, S., Yamaguchi, T., Kusaka, M., Maser, R. L., Takahashi, H., Cowley, B. D. and Grantham, J. J. (2003). Renal activation of extracellular signal-regulated kinase in rats with autosomal-dominant polycystic kidney disease. Kidney Int 63, 427-437.
Nakai, J., Ohkura, M. and Imoto, K. (2001). A high signal-to-noise Ca(2+) probe composed of a single green fluorescent protein. Nat. Biotechnol. 19, 137-141.
Nauli, S. M., Alenghat, F. J., Luo, Y., Williams, E., Vassilev, P., Li, X., Elia, A. E., Lu, W., Brown, E. M., Quinn, S. J. et al. (2003). Polycystins 1 and 2 mediate mechanosensation in the primary cilium of kidney cells. Nat Genet. 33, 129-137.
Nauli, S. M., Rossetti, S., Kolb, R. J., Alenghat, F. J., Consugar, M. B., Harris, P. C., Ingber, D. E., Loghman-Adham, M. and Zhou, J. (2006). Loss of polycystin-1 in human cyst-lining epithelia leads to ciliary dysfunction. J Am Soc Nephrol 17, 1015-1025.
Olson, E. N., Backs, J. and McKinsey, T. A. (2006). Control of cardiac hypertrophy and heart failure by histone acetylation/deacetylation. Novartis Found Symp 274, 3-12; discussion 13-19, 152-155, 272-156.
Otto, E. et al., Nature Reviews Genetics 6, 928 (2005).
Patel, V., Li, L., Cobo-Stark, P., Shao, X., Somlo, S., Lin, F. and Igarashi, P. (2008). Acute kidney injury and aberrant planar cell polarity induce cyst formation in mice lacking renal cilia. Hum Mol. Genet. 2008 17, 1578-1590.
Piontek, K., Menezes, L. F., Garcia-Gonzalez, M. A., Huso, D. L. and Germino, G. G. (2007). A critical developmental switch defines the kinetics of kidney cyst formation after loss of Pkd1. Nat Med 13, 1490-1495.
Praetorius, H. A. and Spring, K. R. (2001). Bending the MDCK cell primary cilium increases intracellular calcium. J Membr Biol 184, 71-79.
Praetorius, H. A., Frokiaer, J., Nielsen, S. and Spring, K. R. (2003). Bending the primary cilium opens Ca2+-sensitive intermediate-conductance K⁺ channels in MDCK cells. J Membr Biol 191, 193-200.
Puri, S., et al., J. Biol. Chem. 279, 55455 (Dec. 31, 2004).
Rossetti, S., Strmecki, L., Gamble, V., Burton, S., Sneddon, V., Peral, B., Roy, S., Bakkaloglu, A., Komel, R., Winearls, C. G. et al. (2001). Mutation analysis of the entire PKD1 gene: genetic and diagnostic implications. Am J Hum Genet. 68, 46-63.
Rubera, I., Poujeol, C., Bertin, G., Hasseine, L., Counillon, L., Poujeol, P. and Tauc, M. (2004). Specific Cre/Lox recombination in the mouse proximal tubule. J Am Soc Nephrol 15, 2050-2056.
Rundle, D. R., Gorbsky, G. and Tsiokas, L. (2004). PKD2 interacts and co-localizes with mDia1 to mitotic spindles of dividing cells: role of mDia1 IN PKD2 localization to mitotic spindles. J Biol Chem 279, 29728-29739.
Sharif-Naeini, R., Folgering, J. H., Bichet, D., Duprat, F., Lauritzen, I., Arhatte, M., Jodar, M., Dedman, A., Chatelain, F. C., Schulte, U. et al. (2009). Polycystin-1 and -2 dosage regulates pressure sensing. Cell 139, 587-596.
Tallini, Y. N., Ohkura, M., Choi, B. R., Ji, G., Imoto, K., Doran, R., Lee, J., Plan, P., Wilson, J., Xin, H. B. et al. (2006). Imaging cellular signals in the heart in vivo: Cardiac expression of the high-signal Ca2+ indicator GCaMP2. Proceedings of the National Academy of Sciences of the United States of America 103, 4753-4758.
van Oort, R. J., van Rooij, E., Bourajjaj, M., Schimmel, J., Jansen, M. A., van der Nagel, R., Doevendans, P. A., Schneider, M. D., van Echteld, C. J. and De Windt, L. J. (2006). MEF2 activates a genetic program promoting chamber dilation and contractile dysfunction in calcineurin-induced heart failure. Circulation 114, 298-308.
Vong, L. H., Ragusa, M. J. and Schwarz, J. J. (2005). Generation of conditional Mef2cIoxP/IoxP mice for temporal- and tissue-specific analyses. Genesis 43, 43-48.
Wang, D. Z., Valdez, M. R., McAnally, J., Richardson, J. and Olson, E. N. (2001). The Mef2c gene is a direct transcriptional target of myogenic bHLH and MEF2 proteins during skeletal muscle development. Development 128, 4623-4633.
Wang, W., Ha, C. H., Jhun, B. S., Wong, C., Jain, M. K. and Jin, Z. G. (2009). Fluid shear stress stimulates phosphorylation-dependent nuclear export of HDAC5 and mediates expression of KLF2 and eNOS. Blood.
Wang, X., Wu, Y., Ward, C. J. and Harris PC, T. V. (2008). Vasopressin directly regulates cyst growth in polycystic kidney disease. J Am Soc Nephrol 19.
Watnick, T. and Germino, G. G. (1999). Molecular basis of autosomal dominant polycystic kidney disease. Semin Nephrol 19, 327-343.
Wilson, P. and Goilav, B. (2007). Cystic disease of the kidney. Annu Rev Pathol. 2.
Wu, G. et al., (1998). Somatic Inactivation of Pkd2 Results in Polycystic Kidney Disease. Cell 93, 177-188.
Wu, G., Markowitz, G. S., Li, L., D'Agati, V. D., Factor, S. M., Geng, L., Tibara, S., Tuchman, J., Cai, Y., Park, J. H. et al. (2000). Cardiac defects and renal failure in mice with targeted mutations in Pkd2. Nat Genet. 24, 75-78.
Xu, J., Gong, N. L., Bodi, I., Aronow, B. J., Backx, P. H. and Molkentin, J. D. (2006). Myocyte enhancer factors 2A and 2C induce dilated cardiomyopathy in transgenic mice. J Biol Chem 281, 9152-9162.
Yamaguchi, T., Hempson, S. J., Reif, G. A., Hedge, A. M. and Wallace, D. P. (2006). Calcium restores a normal proliferation phenotype in human polycystic kidney disease epithelial cells. J Am Soc Nephrol 17, 178-187.
Yamaguchi, T., Nagao, S., Wallace, D. P., Belibi, F. A., Cowley, B. D., Pelling, J. C. and Grantham, J. J. (2003). Cyclic AMP activates B-Raf and ERK in cyst epithelial cells from autosomal-dominant polycystic kidneys. Kidney Int 63, 1983-1994.

What is claimed is:

1. A method of treating or ameliorating an effect of a polycystic disease comprising administering to a patient in need thereof an amount of a modulator of a histone deacetylase (HDAC) pathway, which amount is sufficient to treat or ameliorate an effect of a polycystic disease.

2. The method according to claim 1, wherein the polycystic disease is selected from the group consisting of polycystic kidney disease (PKD), polycystic liver disease (PLD), polycystic ovary syndrome (PCOS), pancreatic cysts, and combinations thereof.

3. The method according to claim 2, wherein the polycystic disease is autosomal dominant polycystic kidney disease (ADPKD) or autosomal recessive polycystic kidney disease (ARPKD).

4. The method according to claim 1, wherein the modulator modulates a class II HDAC.

5. The method according to claim 4, wherein the class II HDAC is HDAC5.

6. The method according to claim 5, wherein the modulator inhibits HDAC5.

7. The method according to claim 1, wherein the modulator of the HDAC pathway is an HDAC inhibitor (HDACi).

8. The method according to claim 7, wherein the HDACi is selected from the group consisting of nucleic acids, polypeptides, polysaccharides, small organic or inorganic molecules, and combinations thereof.

9. The method according to claim 7, wherein the HDACi is selected from the group consisting of Entinostat (Bayer A G, Leverkusen, Germany), KD-5170 (Kalypsys, San Diego, Calif.), KD-5150 (Kalypsys, San Diego, Calif.), KLYP-278 (Kalypsys, San Diego, Calif.), KLYP-298 (Kalypsys, San Diego, Calif.), KLYP-319 (Kalypsys, San Diego, Calif.), KLYP-722 (Kalypsys, San Diego, Calif.), CG-200745 (CrystalGenomics, Inc., Seoul, South Korea), Avugane (TopoTarget AS, København, Denmark), SB-939 (S*BIO, Singapore), ARQ-700RP (ArQule, Woburn, Mass.), KA-001 (Karus Therapeutics, Chilworth, Hampshire, United Kingdom), MG-3290 (MethylGene, Montreal, Quebec, Canada), PXD-118490 (LEO-80140) (TopoTarget AS, København, Denmark), CHR-3996 (Chroma Therapeutics, Abingdon, Oxon, United Kingdom), AR-42 (Arno Therapeutics, Parsippany, N.J.), RG-2833 (RepliGen, Waltham, Mass.), DAC-60 (Genextra, Milan, Italy), SB-1304 (S*BIO, Singapore), SB-1354 (S*BIO, Singapore), 4SC-201 (4SC AG, Planegg-Martinsried, Germany), 4SC-202 (4SC AG, Planegg-Martinsried, Germany), NBM-HD-1 (NatureWise, Biotech & Medicals, Taipei, Taiwan), CU-903 (Curis, Cambridge, Mass.), MG-2856 (MethylGene, Montreal, Quebec, Canada), MG-4230 (MethylGene, Montreal, Quebec, Canada), MG-4915 (MethylGene, Montreal, Quebec, Canada), MG-5026 (MethylGene, Montreal, Quebec, Canada), pharmaceutically acceptable salts thereof, and combinations thereof.

10. The method according to claim 7, wherein the HDACi is selected from the group consisting of hydroxamic acids, short chain fatty acids, cyclic tetrapeptides/epoxides, benzamides, electrophilic ketone derivatives, and combinations thereof.

11. The method according to claim 10, wherein the hydroxamic acid is selected from the group consisting of trichostatin A (TSA), suberoylanilide hydroxamic acid (SAHA), pyroxamide, azelaic-1-hydroxamate-9-anilide (AAHA), compound 13, compound 14, CRA-024781 (Pharmacyclics, Sunnyvale, Calif.), bombesin-2 (BB2) receptor antagonist, JNJ-16241199 (Johnson & Johnson, Langhorne, Pa.), Oxamflatin, CG-1521 (Errant Gene Therapeutics, LLC, Chicago, Ill.), CG-1255 (Errant Gene Therapeutics, LLC, Chicago, Ill.), SK-7068 (In2Gen/SK Chemical Co., Suweon, Korea), SK-7041 (In2Gen/SK Chemical Co., Suweon, Korea), m-carboxycinnamic acid bis-hydroxamide (CBHA), Scriptaid (N-Hydroxy-1,3-dioxo-1H-benz[de]isoquinoline-2(3H)-hexan amide), compound 48, compound 49, compound 50, compound 51, SB-623 (Merrion Research I Limited, National Digital Park, Ireland), SB-639 (Merrion Research I Limited, National Digital Park, Ireland), SB-624 (Merrion Research I Limited, National Digital Park, Ireland), Panobinostat (LBH-589) (Novartis, Basel, Switzerland), NVP-LAQ824 (Novartis, Basel, Switzerland), compound 70, pharmaceutically acceptable salts thereof, and combinations thereof.

12. The method according to claim 10, wherein the short chain fatty acid is selected from the group consisting of butyrate, phenylbutyrate, valporic acid (VPA), Pivanex™ (Titan Pharmaceuticals, Inc.), AN-1 (Titan Pharmaceuticals, Inc.), tributyrin, compound G1, pivaloyloxymethyl butyrate, hyaluronic acid butyric acid ester (HA-But), pharmaceutically acceptable salts thereof, and combinations thereof.

13. The method according to claim 10, wherein the cyclic tetrapeptide/epoxide is selected from the group consisting of Apicidine, Trapoxin-A, Trapoxin-B, cyclic hydroxamic acid-containing peptide 1 (CHAP-1), CHAP-31, CHAP-15, chlamidocin, HC-Toxin, WF-27082B (Fujisawa Pharmaceutical Company, Ltd., Osaka, Japan), Romidepsin (Gloucester Pharmaceuticals, Cambridge, Mass.), Spiruchostatin A, Depudesin, compound D1, Triacetylshikimic acid, Cyclostellettamine FFF1, Cyclostellettamine FFF2, Cyclostellettamine FFF3, Cyclostellettamine FFF4, pharmaceutically acceptable salts thereof, and combinations thereof.

14. The method according to claim 10, wherein the benzamide is selected from the group consisting of MS-27-275 (Schering A G, Germany), Tacedinaline (N-acetyldinaline), compound 27, ITF-2357 (Italfarmaco, Cinisello Balsamo, Italy), compound 29, compound 30, N-hydroxy-4-(3-methyl-2-phenyl-butyrylamino)benzamide (HDAC-42), MGCD-0103 (MethylGene Inc., Montreal, Quebec, Canada), PX-117794 (TopoTarget AS, København, Denmark), compound 37, Belinostat (TopoTarget AS, København, Denmark), Compound 39, sulfonamide hydroxamic acid, pharmaceutically acceptable salts thereof, and combinations thereof.

15. The method according to claim 10, wherein the electrophilic ketone derivative is a trifluoromethyl ketone or an alpha-keto amide.

16. The method according to claim 10, wherein the HDACi is selected from the group consisting of TSA, SAHA, VPA, and combinations thereof.

17. The method according to claim 1, wherein the modulator regulates the activity of a myocyte enhancer factor 2 (MEF2) protein, an heterotrimeric G protein, a phospholipase C(PLC), a protein kinase C(PKC), a protein kinase D (PKD), an inositol 1, 4, 5 triphosphate receptor (IP3R), a calcium calmodulin kinase II (CaMKII), a salt inducible kinase 1 (Sik1), a 14-3-3 polypeptide, a GATA protein, or a MIM protein.

18. The method according to claim 17, wherein the modulator is a MEF2 activator, an heterotrimeric G protein activator, a PLC activator, a PKC activator, a PKD activator, an IP3R activator, a CaMKII activator, a Sik1 activator, or a 14-3-3 activator.

19. The method according to claim 18, wherein the heterotrimeric G protein activator is selected from the group consisting of mastoparan, fluoroaluminate (AlF₄⁻), guanosine 5′—O—(3-thiotriphosphate), G-protein bg (beta gamma) binding peptide mSIRK, MAS 7, Pasteurella multocida toxin, and combinations thereof.

20. The method according to claim 18, wherein the PLC activator is m-3M3FBS.

21. The method according to claim 18, wherein the PKC activator is selected from the group consisting 12-myristate 13-acetate (PMA), phorbol 12,13-dibutyrate (PDBu), phorbol 12,13-didecanoate (PDD), farnesyl thiotriazole, ingenol 3,20-dibenzoate, (−)-7-octylindolactam V, n-heptyl-5-chloronaphthalene-1-sulfonamide, mezerein, ingenol mebutate (Peplin, Emeryville, Calif.), KAI-1455 (KAI Pharmaceuticals, South San Francisco, Calif.), KAI-9706 (KAI Pharmaceuticals, South San Francisco, Calif.), bryostatin-1 nanosome (Aphios, Woburn, Mass.), bryostatin-1, Sapintoxin A, 8-octyl-benzolactam-V9,1-hexylindolactam-V10, phorbol 12-myristate 13-acetate, cholesterol sulfate, daphnoretin, DiC8, farnesyl thiotriazole and combinations thereof.

22. The method according to claim 18, wherein the IP3R activator is adenophostin.

23. The method according to claim 1, wherein the modulator acts upstream of HDAC.

24. The method according to claim 1, wherein the modulator acts downstream of HDAC.

25. A method of treating or ameliorating an effect of a polycystic kidney disease (PKD) comprising administering to a patient in need thereof an amount of an HDAC inhibitor (HDACi) that is sufficient to treat or ameliorate an effect of PKD.

26. The method according to claim 25, wherein the HDACi is selected from the group consisting of hydroxamic acids, short chain fatty acids, cyclic tetrapeptides/epoxides, benzamides, electrophilic ketone derivatives, and combinations thereof.

27. The method according to claim 25, wherein the HDACi is selected from the group consisting of Entinostat (Bayer A G, Leverkusen, Germany), KD-5170 (Kalypsys, San Diego, Calif.), KD-5150 (Kalypsys, San Diego, Calif.), KLYP-278 (Kalypsys, San Diego, Calif.), KLYP-298 (Kalypsys, San Diego, Calif.), KLYP-319 (Kalypsys, San Diego, Calif.), KLYP-722 (Kalypsys, San Diego, Calif.), CG-200745 (CrystalGenomics, Inc., Seoul, South Korea), Avugane (TopoTarget AS, København, Denmark), SB-939 (S*BIO, Singapore), ARQ-700RP (ArQule, Woburn, Mass.), KA-001 (Karus Therapeutics, Chilworth, Hampshire, United Kingdom), MG-3290 (MethylGene, Montreal, Quebec, Canada), PXD-118490 (LEO-80140) (TopoTarget AS, København, Denmark), CHR-3996 (Chroma Therapeutics, Abingdon, Oxon, United Kingdom), AR-42 (Arno Therapeutics, Parsippany, N.J.), RG-2833 (RepliGen, Waltham, Mass.), DAC-60 (Genextra, Milan, Italy), SB-1304 (S*BIO, Singapore), SB-1354 (S*BIO, Singapore), 4SC-201 (4SC AG, Planegg-Martinsried, Germany), 4SC-202 (4SC AG, Planegg-Martinsried, Germany), NBM-HD-1 (NatureWise, Biotech & Medicals, Taipei, Taiwan), CU-903 (Curis, Cambridge, Mass.), MG-2856 (MethylGene, Montreal, Quebec, Canada), MG-4230 (MethylGene, Montreal, Quebec, Canada), MG-4915 (MethylGene, Montreal, Quebec, Canada), MG-5026 (MethylGene, Montreal, Quebec, Canada), trichostatin A (TSA), suberoylanilide hydroxamic acid (SAHA), pyroxamide, azelaic-1-hydroxamate-9-anilide (AAHA), compound 13, compound 14, CRA-024781 (Pharmacyclics, Sunnyvale, Calif.), bombesin-2 (BB2) receptor antagonist, JNJ-16241199 (Johnson & Johnson, Langhorne, Pa.), Oxamflatin, CG-1521 (Errant Gene Therapeutics, LLC, Chicago, Ill.), CG-1255 (Errant Gene Therapeutics, LLC, Chicago, Ill.), SK-7068 (In2Gen/SK Chemical Co., Suweon, Korea), SK-7041 (In2Gen/SK Chemical Co., Suweon, Korea), m-carboxycinnamic acid bis-hydroxamide (CBHA), Scriptaid (N-Hydroxy-1,3-dioxo-1H-benz[de]isoquinoline-2(3H)-hexan amide), compound 48, compound 49, compound 50, compound 51, SB-623 (Merrion Research I Limited, National Digital Park, Ireland), SB-639 (Merrion Research I Limited, National Digital Park, Ireland), SB-624 (Merrion Research I Limited, National Digital Park, Ireland), Panobinostat (LBH-589) (Novartis, Basel, Switzerland), NVP-LAQ824 (Novartis, Basel, Switzerland), compound 70, butyrate, phenylbutyrate, valporic acid (VPA), Pivanex™ (Titan Pharmaceuticals, Inc.), AN-1 (Titan Pharmaceuticals, Inc.), Tributyrin, compound G1, Pivaloyloxymethyl butyrate, Apicidine, Trapoxin-A, Trapoxin-B, cyclic hydroxamic acid-containing peptide 1 (CHAP-1), CHAP-31, CHAP-15, chlamidocin, HC-Toxin, WF-27082B (Fujisawa Pharmaceutical Company, Ltd., Osaka, Japan), Romidepsin (Gloucester Pharmaceuticals, Cambridge, Mass.), Spiruchostatin A, Depudesin, compound D1, Triacetylshikimic acid, hyaluronic acid butyric acid ester (HA-But), Cyclostellettamine FFF1, Cyclostellettamine FFF2, Cyclostellettamine FFF3, Cyclostellettamine FFF4, MS-27-275 (Schering A G, Germany), Tacedinaline (N-acetyldinaline), compound 27, ITF-2357 (Italfarmaco, Cinisello Balsamo, Italy), compound 29, compound 30, N-hydroxy-4-(3-methyl-2-phenyl-butyrylamino)benzamide (HDAC-42), MGCD-0103 (MethylGene Inc., Montreal, Quebec, Canada), PX-117794 (TopoTarget AS, København, Denmark), compound 37, Belinostat (TopoTarget AS, København, Denmark), Compound 39, sulfonamide hydroxamic acid, trifluoromethyl ketone, an alpha-keto amide, pharmaceutically acceptable salts thereof, and combinations thereof.

28. The method according to claim 25, wherein the HDACi inhibits a class II HDAC.

29. The method according to claim 25, wherein the polycystic kidney disease is autosomal dominant polycystic kidney disease (ADPKD) or autosomal recessive polycystic kidney disease (ARPKD).

30. A method of treating or ameliorating an effect of a polycystic kidney disease (PKD) comprising administering to a patient in need thereof an amount of an HDAC5 inhibitor that is sufficient to treat or ameliorate an effect of PKD.

31. The method according to claim 30, wherein the polycystic kidney disease is autosomal dominant polycystic kidney disease (ADPKD) or autosomal recessive polycystic kidney disease (ARPKD).


SEQUENCE LISTING (26 sequences)

SEQ ID NO: 1 - DNA, 1410 bp, Artificial
  Synthetic construct; FLAG-MEF2C
atggattacaaagaccatgacggtgattataaagatcatgacatcgactacaaagacgatgacgacaagcttgcggccgcgaattcatcgatagatctgatatcggtaccaatggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggaggcattgaacaagaaagaaaacaaaggctctgaaagccccgatcctgactcctcttatgcactcaccccacgcactgaagaaaaatacaaaaaaattaatgaagaatttgataatatgatcaagagtcataaaattcctgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacataa

SEQ ID NO: 2 - DNA, 1299 bp, Mus musculus
atggggagaaaaaagattcagattacgaggataatggatgagcgtaacagacaggtgacttttacgaagaggaaatttggattgatgaagaaggcttatgagctgagcgtgctgtgcgactgtgagattgcactgatcatcttcaacagcaccaacaagctgttccagtacgccagcactgacatggataaggtgttgctcaagtacaccgagtacaacgagccgcacgagagccggacaaactcagacattgtggaggcattgaacaagaaagaaaacaaaggctctgaaagccccgatcctgactcctcttatgcactcaccccacgcactgaagaaaaatacaaaaaaattaatgaagaatttgataatatgatcaagagtcataaaattcctgctgttccacctcccagctttgagatgccagttaccatcccagtgtccagccataacagtttggtgtacagcaatcctgtcagcacactgggaaaccccaatcttctgccactggcccacccgtctctgcagaggaatagtatgtctcctggtgtaacacatagacctccaagtgcaggtaacacaggcggtctgatgggcggagatctgacatccggtgcaggcaccagcgcagggaatggatacggcaacccccggaactcaccaggcctgctggtctcacctggtaacctgaacaagaatatacaagccaaatctcctccccctatgaatctaggaatgaataatcgtaagccagatctccgcgttcttatcccacctggcagcaagaacacgatgccatcagtgaatcaaaggataaataactcccagtcggctcagtcattggctaccccggtggtttccgtagcaactcctactttaccaggacaaggaatgggaggatatccatcagccatttcaacaacatatggtactgagtactctctgagtagcgcagatctgtcatctctgtctggcttcaacactgccagtgcgctccacctcggctctgtaactggctggcagcagcagcacctacataacatgccgccatctgccctcagtcagttgggagaccgtaccaccaccccttcgagatacccacaacacaccacgcgccacgaggcggggaggtctcctgttgacagcttgagcagctgtagcagttcctacgatgggagcgaccgagaggatcaccggaacgaattccactcccccattggactcaccagaccttcgccggacgaaagggaaagtccttcagtcaagcgcatgcgactctctgaaggatgggcaacataa

SEQ ID NO: 3 - DNA, 4113 bp, Artificial
  Synthetic Construct; HDAC5-GFP
atgaactctcccaacgagtcggatggcatgtcaggccgggaaccatccttggaaatcctgccacggactcctctgcacagcatccctgtggcagtggaggtgaagccggtgctgccaggagccatgcccagctccatggggggtggaggtggaggtagccccagccccgtggagcttcggggggctctggcgggccccatggaccctgcgctacgggagcagcaactgcagcaggagctcctggtcctcaagcagcagcagcagctccagaagcagctcctgttcgccgagttccagaagcagcacgaccacttgacgcggcagcacgaggtccagctgcagaagcacctcaagcagcagcaggagatgctggcggctaagaggcagcaggagctggagcagcagcggcagcgggagcagcagcggcaggaggagctggagaaacagcggctggagcagcagctgctcatcctgcgcaacaaggagaagagcaaagagagtgccatcgccagcaccgaggtaaagctgaggctccaggaattcctgttgtccaagtcaaaggagcccacgccaggcggcctcaaccattccctcccacagcaccccaaatgctggggagcccaccacgcttctttggaccagagttcccctccccagagcggccctcctgggacgcctccctcctacaaattgcctttgcttgggccctatgacagccgtgatgactttcccctccgtaaaacggcctcggaacccaacttaaaagtacgttcgaggctaaaacagaaggtagccgagaggagaagcagtcccctcctgcgtcgaaaggatggcactgttattagtacttttaagaagagagcagttgagatcaccggcacggggcctggggtgtcgtccgtgtgtaacagtgcgcccggctctggccccagctctcccaacagttcccacagcaccatcgctgagaacggctttactggctcagtccccaacatccccactgagatgatcccccagcaccgggccctccctctggacagttccccaaaccagttcagcctctatacgtctccttctctgcccaacatctccctagggctgcaggccactgtcactgtcaccaactcgcacctcaccgcctccccgaagctgtcaacacagcaggaggctgagaggcaggcccttcagtccctgcggcagggcggcacactgaccggcaagttcatgagcacatcctccatccctggctgcctgttgggagtggcactggagggtgacacaagcccccacgggcacgcttccctgctgcagcacgttttgctcctggagcaggcccggcaacagagcacgctcatagcagtgccgctccatgggcagtccccactggtgacgggtgaacgtgtggccaccagcatgaggacggtgggtaagctcccgaggcaccgacctctgagccgcactcagtcctccccgctgccgcagagtccccaggccctgcagcagctggtcatgcagcagcagcaccagcagttcctggagaagcagaagcagcagcagatgcagctgggcaagatccttaccaaaactggggagctgtcaaggcagcccaccactcacccggaggagacagaagaggagctgacggagcagcaggaggccttgctgggagagggggccctgaccattccccgggaaggctctacagaaagtgagagcacccaggaagacctagaagaggaggaggaggaggaggaggaggaagaggaggactgcattcaggtcaaggatgaggatggcgagagtggtcctgatgaaggccctgacttagaagagtccagtgctggttacaaaaagttgttcgcagatgcccagcagttacagcccctccaggtgtaccaggcacccctcagcctggccactgtgcctcatcaggccctgggccgcacccagtcctcacctgctgctcctgggagcatgaagagccccacagaccaacccactgtggtgaagcacctcttcaccacaggtgtggtctatgacacgttcatgctgaagcaccagtgtatgtgcggaaacacacacgtgcacccagagcacgccggccgcatccagagcatctggtcccggctgcaggaaactggtctgctcggcaagtgtgagcggatccggggtcgtaaagccacactggatgaaatccagaccgtgcactctgagtaccacaccctgctctatgggaccagcccccttaaccggcagaagctggacagcaagaagctgcttggccccatcagccagaagatgtacgccatgctgccctgtgggggcattggggtggacagtgacacggtgtggaatgagatgcactcctcaagtgccgtgcgaatggcagtgggctgcctggtggagctggccttcaaggtggctgcaggagagctcaagaatggatttgctatcatccggcccccaggacaccatgctgaggagtccacagccatgggattctgcttcttcaactccgtagccatcacagctaaactcctgcagcagaagctgagcgtgggcaaggtcctcatcgtggactgggatattcaccatggcaacggcacccagcaagcattctacaacgatccctctgtgctctacatctccctgcatcgctacgacaacgggaacttctttccaggctctggggctcctgaagaggttggtggagggccaggtgtggggtacaacgtaaatgtggcgtggacaggaggtgtggatccccccattggagatgtggaatacctgacagccttcaggacagtggtgatgcccattgcccaggagttctcacctgacgtcgtcctagtctccgctgggtttgatgctgttgaaggacatctgtctccactgggtggctattctgtcaccgccagatgttttggccacttgaccaggcagctcatgacactggctgggggccgggtggtgctggccctggagggaggccatgacttgaccgccatctgtgatgcctctgaggcctgtgtctcggctctgctcagcgtggagctgcagcccttggatgaagcagtcttgcagcaaaagcccagcgtcaatgcggttgccacactagagaaagtcatcgagatccagagcaaacactggagctgtgtacagaggtttgccgctggtctgggctgctcgctgcgggaggctcagacaggtgagaaagaggaggccgagactgtgagcgccatggccctgctttccgtgggggctgagcaggcccaggctgttgccactcaagagcacagccccaggccagcagaggagcccatggagcaggagcctgccctgaagcttcgaattctgcagtcgacggtaccgcgggcccgggatccaccggtcatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtga

SEQ ID NO: 4 - DNA, 3342 bp, Mus musculus
atgaactctcccaacgagtcggatggcatgtcaggccgggaaccatccttggaaatcctgccacggactcctctgcacagcatccctgtggcagtggaggtgaagccggtgctgccaggagccatgcccagctccatggggggtggaggtggaggtagccccagccccgtggagcttcggggggctctggcgggccccatggaccctgcgctacgggagcagcaactgcagcaggagctcctggtcctcaagcagcagcagcagctccagaagcagctcctgttcgccgagttccagaagcagcacgaccacttgacgcggcagcacgaggtccagctgcagaagcacctcaagcagcagcaggagatgctggcggctaagaggcagcaggagctggagcagcagcggcagcgggagcagcagcggcaggaggagctggagaaacagcggctggagcagcagctgctcatcctgcgcaacaaggagaagagcaaagagagtgccatcgccagcaccgaggtaaagctgaggctccaggaattcctgttgtccaagtcaaaggagcccacgccaggcggcctcaaccattccctcccacagcaccccaaatgctggggagcccaccacgcttctttggaccagagttcccctccccagagcggccctcctgggacgcctccctcctacaaattgcctttgcttgggccctatgacagccgtgatgactttcccctccgtaaaacggcctcggaacccaacttaaaagtacgttcgaggctaaaacagaaggtagccgagaggagaagcagtcccctcctgcgtcgaaaggatggcactgttattagtacttttaagaagagagcagttgagatcaccggcacggggcctggggtgtcgtccgtgtgtaacagtgcgcccggctctggccccagctctcccaacagttcccacagcaccatcgctgagaacggctttactggctcagtccccaacatccccactgagatgatcccccagcaccgggccctccctctggacagttccccaaaccagttcagcctctatacgtctccttctctgcccaacatctccctagggctgcaggccactgtcactgtcaccaactcgcacctcaccgcctccccgaagctgtcaacacagcaggaggctgagaggcaggcccttcagtccctgcggcagggcggcacactgaccggcaagttcatgagcacatcctccatccctggctgcctgttgggagtggcactggagggtgacacaagcccccacgggcacgcttccctgctgcagcacgttttgctcctggagcaggcccggcaacagagcacgctcatagcagtgccgctccatgggcagtccccactggtgacgggtgaacgtgtggccaccagcatgaggacggtgggtaagctcccgaggcaccgacctctgagccgcactcagtcctccccgctgccgcagagtccccaggccctgcagcagctggtcatgcagcagcagcaccagcagttcctggagaagcagaagcagcagcagatgcagctgggcaagatccttaccaaaactggggagctgtcaaggcagcccaccactcacccggaggagacagaagaggagctgacggagcagcaggaggccttgctgggagagggggccctgaccattccccgggaaggctctacagaaagtgagagcacccaggaagacctagaagaggaggaggaggaggaggaggaggaagaggaggactgcattcaggtcaaggatgaggatggcgagagtggtcctgatgaaggccctgacttagaagagtccagtgctggttacaaaaagttgttcgcagatgcccagcagttacagcccctccaggtgtaccaggcacccctcagcctggccactgtgcctcatcaggccctgggccgcacccagtcctcacctgctgctcctgggagcatgaagagccccacagaccaacccactgtggtgaagcacctcttcaccacaggtgtggtctatgacacgttcatgctgaagcaccagtgtatgtgcggaaacacacacgtgcacccagagcacgccggccgcatccagagcatctggtcccggctgcaggaaactggtctgctcggcaagtgtgagcggatccggggtcgtaaagccacactggatgaaatccagaccgtgcactctgagtaccacaccctgctctatgggaccagcccccttaaccggcagaagctggacagcaagaagctgcttggccccatcagccagaagatgtacgccatgctgccctgtgggggcattggggtggacagtgacacggtgtggaatgagatgcactcctcaagtgccgtgcgaatggcagtgggctgcctggtggagctggccttcaaggtggctgcaggagagctcaagaatggatttgctatcatccggcccccaggacaccatgctgaggagtccacagccatgggattctgcttcttcaactccgtagccatcacagctaaactcctgcagcagaagctgagcgtgggcaaggtcctcatcgtggactgggatattcaccatggcaacggcacccagcaagcattctacaacgatccctctgtgctctacatctccctgcatcgctacgacaacgggaacttctttccaggctctggggctcctgaagaggttggtggagggccaggtgtggggtacaacgtaaatgtggcgtggacaggaggtgtggatccccccattggagatgtggaatacctgacagccttcaggacagtggtgatgcccattgcccaggagttctcacctgacgtcgtcctagtctccgctgggtttgatgctgttgaaggacatctgtctccactgggtggctattctgtcaccgccagatgttttggccacttgaccaggcagctcatgacactggctgggggccgggtggtgctggccctggagggaggccatgacttgaccgccatctgtgatgcctctgaggcctgtgtctcggctctgctcagcgtggagctgcagcccttggatgaagcagtcttgcagcaaaagcccagcgtcaatgcggttgccacactagagaaagtcatcgagatccagagcaaacactggagctgtgtacagaggtttgccgctggtctgggctgctcgctgcgggaggctcagacaggtgagaaagaggaggccgagactgtgagcgccatggccctgctttccgtgggggctgagcaggcccaggctgttgccactcaagagcacagccccaggccagcagaggagcccatggagcaggagcctgccctg

SEQ ID NO: 5 - DNA, 23 bp, Artificial
  Synthetic Construct; MIM forward
ccagccagggttgccatagccac

SEQ ID NO: 6 - DNA, 29 bp, Artificial
  Synthetic Construct; MIM reverse
tgactcttgcaaacggttcaattaggtgc

SEQ ID NO: 7 - RNA, 19 nt, Artificial
  Synthetic Construct; MEF2C RNAi
gaggaucaccggaacgaau

SEQ ID NO: 8 - RNA, 19 nt, Artificial
  Synthetic Construct; MEF2C RNAi
uaguaugucuccuggugua

SEQ ID NO: 9 - RNA, 19 nt, Artificial
  Synthetic Construct; MEF2C RNAi
gauaauggaugagcguaac

SEQ ID NO: 10 - RNA, 19 nt, Artificial
  Synthetic Construct; MEF2C RNAi
ccagaucuccgcguucuua

SEQ ID NO: 11 - DNA, 20 bp, Artificial
  Synthetic Construct; mef2c forward
cctggcagcaagaacacgat

SEQ ID NO: 12 - DNA, 22 bp, Artificial
  Synthetic Construct; mef2c reverse
gactgagccgactgggagttat

SEQ ID NO: 13 - DNA, 16 bp, Artificial
  Synthetic Construct; mef2c probe
ccatcagtgaatcaaa

SEQ ID NO: 14 - DNA, 22 bp, Artificial
  Synthetic Construct; mim forward; qpcr
gctcagctctcaccagaatgtg

SEQ ID NO: 15 - DNA, 22 bp, Artificial
  Synthetic Construct; mim rev; qpcr
cgatcaaagcactggagaactg

SEQ ID NO: 16 - DNA, 14 bp, Artificial
  Synthetic Construct; mim probe
atgcggcaccggag

SEQ ID NO: 17 - DNA, 18 bp, Artificial
  Synthetic Construct; HDAC5 forward
gctcatcctgcgcaacaa

SEQ ID NO: 18 - DNA, 21 bp, Artificial
  Synthetic Construct; HDAC5 reverse
gaattcctggagcctcagctt

SEQ ID NO: 19 - DNA, 17 bp, Artificial
  Synthetic Construct; HDAC5 probe
caaagagagtgccatcg

SEQ ID NO: 20 - DNA, 18 bp, Artificial
  Synthetic Construct; GATA6 forward
gctcatcctgcgcaacaa

SEQ ID NO: 21 - DNA, 21 bp, Artificial
  Synthetic Construct; GATA6 reverse
gaattcctggagcctcagctt

SEQ ID NO: 22 - DNA, 17 bp, Artificial
  GATA6 probe
caaagagagtgccatcg

SEQ ID NO: 23 - RNA, 19 nt, Artificial
  Synthetic Construct
gaggaucaccggaacgaau

SEQ ID NO: 24 - RNA, 19 nt, Artificial
  Synthetic Construct
uaguaugucuccuggugua

SEQ ID NO: 25 - RNA, 19 nt, Artificial
  Synthetic Construct
gauaauggaugagcguaac

SEQ ID NO: 26 - RNA, 19 nt, Artificial
  Synthetic Construct
ccagaucuccgcguucuua